SEQUENCE LISTING (9 sequences)

SEQ ID NO: 1 - DNA, 12 bp, Artificial Sequence
  Synthetic
actgactgactg

SEQ ID NO: 2 - DNA, 12 bp, Artificial Sequence
  Synthetic
acgtacgtacgt

SEQ ID NO: 3 - protein, 5 aa, Artificial Sequence
  Synthetic
  MISC_FEATURE (2)..(2): is modified
GKRFT

SEQ ID NO: 4 - DNA, 39 bp, Artificial Sequence
  Synthetic
tttttttttttggaattttttttttggaatttttttttt

SEQ ID NO: 5 - DNA, 21 bp, Artificial Sequence
  Synthetic
aaaaaaaaaaacaagaataaa

SEQ ID NO: 6 - DNA, 21 bp, Artificial Sequence
  Synthetic
aacaacacaaccacgactcaa

SEQ ID NO: 7 - DNA, 21 bp, Artificial Sequence
  Synthetic
aagaagagaagcaggagtgaa

SEQ ID NO: 8 - DNA, 21 bp, Artificial Sequence
  Synthetic
aataatataatcatgatttaa

SEQ ID NO: 9 - DNA, 21 bp, Artificial Sequence
  Synthetic
acaacacaacaccagcataaa

(19) United States
(12) Patent Application Publication
(10) Pub. No.: US 2022/0213541 A1
(43) Pub. Date: Jul. 7, 2022

(54) METHOD FOR DETERMINING A POLYMER SEQUENCE

(71) Applicant: Oxford Nanopore Technologies Limited, Oxford (GB)

(72) Inventors: Clive Gavin Brown, Cambridge (GB); Timothy Lee Massingham, Oxford (GB); Stuart William Reid, Oxford (GB)

(73) Assignee: Oxford Nanopore Technologies Limited, Oxford (GB)

(21) Appl. No.: 17/272,986

(22) PCT Filed: Sep. 4, 2019
(86) PCT No.: PCT/GB2019/052456

§ 371 Date: Mar. 3, 2021

(30) Foreign Application Priority Data
Sep. 4, 2018 (GB) 1814369.3

(51) Int. Cl. C12Q 1/6869; G16B 40/10; G16B 40/20

(52) U.S. Cl. CPC C12Q 1/6869; G16B 40/20; G16B 40/10

(57) ABSTRACT

The invention resides in a method of determining a sequence of a target polymer, or part thereof, comprising polymer units comprising canonical and non-canonical polymer units. The method comprises taking a series of measurements of a signal relating to the target polymer wherein a measurement of the signal is dependent upon a plurality of polymer units, and wherein the polymer units of the target polymer modulate the signal, and wherein a non-canonical polymer unit modulates the signal differently from a corresponding canonical polymer unit. The series of measurements are analysed using a machine learning technique that attributes a measurement of a non-canonical polymer unit to being a measurement of a respective corresponding canonical polymer unit. The sequence of the target polymer, or part thereof, is determined from the analysed series of measurements. A non-canonical polymer unit identified from the analysis can be additionally or alternatively determined. Two or more types of non-canonical polymer units corresponding to the two or more types of canonical polymer unit can be used. The polynucleotide can be DNA.

### RELATED APPLICATIONS

This application is a national stage filing under 35 U.S.C. § 371 of international application number PCT/GB2019/052456, filed Sep. 4, 2019, which claims the benefit of Great Britain application number GB 1814369.3, filed Sep. 4, 2018, each of which is herein incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 23, 2021, is named 0036670113US00-SEQ-KZM and is 2,009 bytes in size.

The present invention relates to methods of determining a polymer sequence and to the analysis of measurements taken from polymer units in one or more polymers, for example but without limitation a polynucleotide, during translocation of the polymer with respect to a nanopore. Aspects of the invention relate to the preparation of a polymer for use in such methods, and the determination of a consensus sequence.

A type of measurement system for estimating a target sequence of polymer units in a polymer uses a nanopore, and the polymer is translocated with respect to the nanopore. Some property of the system depends on the polymer units in the nanopore, and measurements of that property are taken. This type of measurement system using a nanopore has been shown to be highly effective, particularly in the field of sequencing a polynucleotide such as DNA or RNA, and has been the subject of much recent development. More recently, this type of measurement system using a nanopore has been shown to be highly effective, particularly in the field of sequencing peptide polymers such as proteins (Nivala et al., 2013 Nat. Biotech.).

Such nanopore measurement systems can provide long continuous reads of polynucleotides ranging from hundreds to hundreds of thousands (and potentially more) nucleotides. The data gathered in this way comprise measurements, such as measurements of ion current, where each translocation of the sequence with respect to the sensitive part of the nanopore can result in a change in the measured property.

The signal measured during movement of a polynucleotide with respect to a nanopore, such as for example translocation of the polymer through a nanopore, has been shown to be dependent upon plural nucleotides and is complex. Analytical techniques of estimating a polymer sequence from measurements taken during interaction of the polynucleotide with a nanopore include the use of a Hidden Markov Model (HMM) such as disclosed in PCT/GB2012/052343. Machine learning techniques such as a recurrent neural network may also be employed and are particularly useful for determining long range information. Such a technique is disclosed in PCT/GB2018/051208, hereby incorporated by reference in its entirety.

Methods comprising analysing the series of measurements using a machine learning technique are known. Such methods include deriving a series of posterior probability matrices corresponding to respective measurements or respective groups of measurements, each posterior probability matrix representing, in respect of different respective historical sequences of polymer units corresponding to measurements prior or subsequent to the respective measurement, posterior probabilities of plural different changes to the respective historical sequence of polymer units giving rise to a new sequence of polymer units.

Improving the accuracy of the analysis of a polymer that has translocated through a nanopore, particularly on long reads of a polymer, often has a high computational expense.

A number of methods for determining the sequence of a polynucleotide have been described in which a modified polynucleotide is generated based on a template polynucleotide sequence.

WO 2015/124935, incorporated by reference herein in its entirety, describes methods for characterising a template polynucleotide using a polymerase to prepare a modified polynucleotide which is subsequently characterised. The modified polynucleotide is prepared such that the polymerase replaces one or more of the nucleotide species in the template polynucleotide with a different nucleotide species when forming the modified polynucleotide. WO 2015/124935 also describes a method of characterising a homopolynucleotide by forming a modified polynucleotide using a polymerase, in which the polymerase when forming the modified polynucleotide randomly replaces some of the instances of the nucleotide species that is complementary to the nucleotide species in the homopolynucleotide with a different nucleotide species.

The invention generally resides in a method of determining a sequence of a target polymer, or part thereof, comprising different types of polymer unit. The method involves taking a series of measurements of a signal relating to the target polymer. These measurements can be obtained or retrieved, or be derived from passing the target polymer strand through a nanopore. The measured signal is dependent upon a plurality of polymer units. For example, the signal measured in respect of the movement of a plurality of polymer units through a nanopore. The polymer units of the target polymer modulate the signal.

A polymer may comprise canonical and non-canonical polymer units. A non-canonical polymer unit typically modulates the signal differently from a corresponding canonical polymer unit. By way of example, in the case of nucleic acids, these corresponding canonical polymer units can be a matched polymer unit e.g. a modified C can correspond to a canonical C, or the identification of a universal nucleotide (for example a universal nucleotide as described herein) can correspond to any one of the canonical values C, A, G or T.

For example, the signal of the target polymer can be attributed to the polymer units ‘CcAGT’, wherein ‘c’ is a modified ‘C’ and the otherwise identical polymer units are canonical only components, namely CCAGT. The signal can include and measure the non-canonical units and during the analysis, or subsequent to the analysis, the non-canonical units can be construed or recognised as a canonical unit. In other words, an alternative base, such as a non-canonical base can be labelled as a canonical base.

A polymer may comprise canonical and non-canonical polymer units. A non-canonical polymer unit typically modulates the signal differently from a corresponding canonical polymer unit. By way of example, in a polypeptide these corresponding canonical polymer units can be a matched polymer unit i.e. a modified Lys can correspond to a canonical Lys.

For example, the signal of the target polymer can be attributed to the polymer units ‘Gly-Lys*-Arg-Phe-Thr’(SEQ ID NO: 3), wherein ‘Lys*’ is a modified ‘Lys’ and the otherwise identical polymer units are canonical-only components. The signal can include and measure the non-canonical units, and during the analysis, or subsequent to the analysis, the non-canonical units can be construed or recognised as a canonical unit. In other words, an alternative amino acid, such as a non-canonical amino acid can be labelled as a canonical amino acid.

In some embodiments, a polypeptide comprising one or more non-canonical amino acids may be prepared by chemical conversion of one or more canonical amino acid to a corresponding non-canonical amino acid. By way of example, a polypeptide comprising canonical amino acids may be contacted with a chemical capable of converting one or more types of canonical amino acids to a corresponding non-canonical amino acid type. Examples of such chemicals include amine reactive groups, such as NHS esters, and thiol reactive groups such as maleimides.

In some embodiments, a polypeptide comprising one or more non-canonical amino acids may be prepared by enzymatic conversion of one or more canonical amino acid to a corresponding non-canonical amino acid. By way of example, a polypeptide comprising canonical amino acids may be contacted with an enzyme capable of converting one or more types of canonical amino acids to a corresponding non-canonical amino acid type. Examples of such enzymes include kinases, phosphatases, transferases and ligases, which add or remove functional groups, proteins, lipids or sugars to or from amino acid side chains.

The method analysing the series of measurements uses a machine learning technique. The machine learning technique can include training. The machine learning technique attributes a measurement of one type of polymer unit to be a measurement of a different type of polymer unit. For example, a non-canonical ‘c’ can be recognised as a canonical ‘C’.

The method further determines the sequence of the target polymer, or part thereof, from the analysed series of measurements, wherein the sequence is expressed as a reduced number of different types of polymer unit.

The methods of the invention can, in particular, focus upon parts or sub-regions of the target polymer. These sub-regions can be areas of interest and/or be subject to a deeper level of analysis. Such parts or sub-regions can include homopolymer regions. Homopolymer regions, and other such areas of interest, of original polymers tend to have low levels of complexity or variation that tends to lead to low variations in the signals derived therefrom.

Having non-canonical units in the target polymer increases the levels of complexity or variation in the signals derived therefrom.

The method can perform analysis to identify non-canonical polymer units and use the combination of canonical and non-canonical information to improve the accuracy of the determined sequence. If the method attributes a measurement of a non-canonical polymer unit to one type of polymer unit, or one of a selection of polymer units, then the accuracy of the sequenced determined from the target polymer is improved because the measurement output is based only upon canonical polymer units, which in turn reduces the computational power required to generate the single-read base-calls and/or the alignment and/or the consensus.

In a particular aspect, the machine learning technique method may attribute a measurement of a non-canonical polymer unit to be a measurement of a corresponding canonical polymer unit. Thus a non-canonical base is base-called as its corresponding canonical base. This has a lower computational requirement compared wherein the machine learning technique is trained to recognise and base-call both the canonical base and the non-canonical base. Attributing a measurement of a non-canonical polymer unit to being a measurement of a corresponding canonical polymer unit can also lead to an overall increase in sequencing accuracy compared to where the machine learning technique is trained to only recognise and base-call canonical bases. In the latter case measurements of a non-canonical bases can result in sequencing errors as they are not recognised by the base-caller.

According to an aspect of the present invention, there is provided a method of determining a sequence of a target polymer comprising polymer units comprising canonical bases and non-canonical polymer units.

The canonical bases can, for example, be A,G,C,T for DNA. A plurality of non-canonical polymer units can be used. A plurality of types of non-canonical polymer units can be used.

The target polymer can be synthesised from an original naturally-occurring polymer. The target polymer can be derived from an original polymer in which a proportion of canonical polymer units have been substituted with alternative polymer units in a non-deterministic manner. Alternatively, the target polymer can be a naturally-occurring polymer having naturally occurring non-canonical polymer units or bases.

The method comprises (i) taking a series of measurements of a signal relating to the target polymer, wherein a measurement of the signal, which can be the measured signal, is dependent upon a plurality of polymer units, and wherein the polymer units of the target polymer modulate the signal, and wherein a non-canonical polymer unit modulates the signal differently from a corresponding canonical polymer unit, (ii) analysing the series of measurements using a machine learning technique, which has preferably been trained, that attributes a measurement of a non-canonical polymer unit to being a measurement of a respective corresponding canonical polymer unit, and (iii) determining the sequence of the target polymer from the analysed series of measurements.

Non-canonical polymer units, or alternative bases, can include by way of example methylated-nucleotides, inosine, bridged-nucleotides and artificial bases.

The corresponding canonical polymer units can be a matched polymer unit i.e. c to C, or can be one of a set of polymer units, wherein, for example, inosine can correspond to any one of the canonical bases C, A, G or T.

For example, when analysing the measurement a non-canonical ‘c’ can be recognised as such and/or recognised as a canonical ‘C’.

When a non-canonical ‘c’ can be recognised as a canonical ‘C’, the invention can provide a way to provide a signal with more information by also measuring alternative bases without needing to make a base-call of those alternative bases thus making it computationally less expensive than if all the non-canonical bases were determined. The base-caller does not make a determination of whether a particular base is canonical or non-canonical in nature.

The method can also accommodate target polymers having a non-naturally corresponding canonical base—for example X is expressed as C, or TT dimer expressed as T.

A non-canonical polymer unit identified from the analysis can additionally or alternatively retain a measurement of a non-canonical polymer unit as being a measurement of a respective corresponding canonical polymer unit. This information on the identity and sequence position of a non-canonical polymer can be kept or stored for use for scoring or weighting during subsequent analysis or determination of a sequence.

Determining a sequence of a target polymer can involve different variations on base calls. For example, if the target polymer had four canonical bases A, C, G and T and four corresponding non-canonical bases a, c, g and t, then the base caller could call only the canonical bases i.e. four (4) bases from eight (8).

If, for example, the target polymer had four canonical bases A, C, G and T and four corresponding non-canonical bases a, c, g and t, wherein the ‘c’ was a methylated-C then the base caller could call five (5) bases being the canonical bases and methylated-C, i.e. five (4) bases from eight (8).

The target polymer can comprise two or more types of non-canonical polymer units corresponding to the two or more types of canonical polymer unit. For example, the target polymer has four canonical bases A, C, G and T and two or more alternative bases.

The identity and sequence position of a non-canonical polymer unit can be determined. That is, where a non-canonical base is called, for example 5 out of 8.

The target polymer can be a polynucleotide.

The target polymer can comprise non-canonical polymer units corresponding to each type of canonical polymer unit. For example the four canonical bases A, C, G and T in addition to four corresponding non-canonical bases a, c, g and t.

The machine learning technique can, alternatively, not determine whether a polymer unit is non-canonical. The analysis and sequence can produce only canonical bases.

The target polymer can comprise plural non-canonical polymer units for each of the one or more types of non-canonical polymer unit present. For example, the target polymer has four canonical bases A, C, G and T and eight corresponding non-canonical bases a, a′, c, c′, g, g′, t and t′. The base caller could call the canonical bases i.e. four (4) bases from twelve (12).

A non-canonical polymer unit can correspond to more than one canonical polymer unit. For example, inosine can base-pair with more than one canonical base—non-specific binding.

The target polymer can comprise from 1 unit to approximately 50% of non-canonical polymer units. 50% provides the maximum amount of disruption by modified bases.

A non-canonical polymer unit can be a modified canonical polymer unit, for example methylated C.

The non-canonical polymer unit can be naturally modified. For example, it occurs naturally in vivo and has not been specifically introduced.

The series of measurements can be taken during movement of the target polymer with respect to a nanopore.

The measurements can be measurements indicative of ion current flow through the nanopore or measurements of a voltage across the nanopore during translocation of the target polymer.

The machine learning technique can be trainable by a method comprising the steps of: providing a plurality of target polymers, for example training strands, comprising non-canonical units that have been substituted for equivalent canonical units at varying sequence positions in the target polymer; taking series of measurements of signals relating to the target polymers; analysing the series of measurements using the machine learning technique; and estimating the corresponding canonical polymer units of the polymer training strands, which can be the underlying sequence.

The machine learning technique can incorporate at least one of a recurrent neural network, a convolutional neural network, a transformer network, attention mechanism, random forests, support vector machines, a restricted Boltzmann machine, hidden Markov model, Markov random field, conditional random field, or a combination thereof.

The polymer can be chosen from a polynucleotide, a polypeptide or a polysaccharide. In particular the polymer is a polynucleotide and the polymer units can be nucleotide bases.

The one or more non-canonical bases can be modified by means of an enzyme.

The method can further comprise the step of modifying a canonical polymer to provide the target polymer comprising one or more one or more non-canonical bases of one or more different types.

A method according to any preceding claim, wherein the polynucleotide comprising one or more non-canonical bases of one or more different types is generated from its complement by use of a polymerase and a proportion of non-canonical bases.

The polynucleotide can be DNA. The movement of the polynucleotide with respect to the nanopore can be controlled by an enzyme. The enzyme can be a helicase. A target polymer training strand can comprise more than one type of non-canonical polymer unit.

According to another aspect of the present invention, there is provided a method of determining a consensus sequence of a target polymer comprising: providing a plurality of polymers wherein the polymers comprise canonical polymer units and non-canonical polymer units, and each of the polymers comprises a region of polymer units that corresponds to a region of the target polymer; analysing measurements of signals relating to the plurality of polymers, wherein a measurement is dependent upon plural polymer units, and wherein the polymer units of the target polymer modulate the signal, and wherein a non-canonical polymer unit modulates the signal differently from a corresponding canonical polymer unit; and determining a consensus sequence from the analysed series of measurements of the plurality of polymers.

A polymer (for example, a polynucleotide) may comprise a region of polymer units (for example a region of nucleotides) that corresponds to a region of another polymer (for example, a region of a target polymer, e.g. a target polynucleotide).

A region of polymer units that “corresponds to” a region of another polymer may have a sequence that is the same as, or complementary to, the sequence of the corresponding region, taking the presence of non-canonical polymer units into account such that the presence of a non-canonical polymer unit is taken to represent a corresponding canonical polymer unit. Thus, a polymer region comprising canonical polymer units may correspond to a polymer region comprising one or more corresponding non-canonical polymer units. By way of example, a skilled person would consider that a polymer region having a specific sequence of canonical polymer units corresponded to an otherwise identical polymer region in which one or more of the canonical polymer units were replaced by corresponding non-canonical polymer units.

A region of polymer units that “corresponds to” a region of another polymer may have a sequence that can be aligned with the sequence of the corresponding region. Methods for the alignment of polymer sequences (for example, the alignment of polynucleotide sequences) are well known in the art, for example sequence alignment programs, and would be familiar to a skilled person. A region may align directly with a corresponding region, or a region may align with a complementary sequence of a corresponding region (for example, a complementary polynucleotide sequence). A skilled person would readily appreciate that the nature of canonical polymer units and corresponding non-canonical polymer units means that a polymer region comprising canonical polymer units may be aligned with a corresponding polymer region comprising one or more corresponding non-canonical units.

Two regions of polymer (e.g. polynucleotide) that correspond to each other may be homologous.

Analysing the series of measurements can comprise a machine learning technique that attributes a measurement of a non-canonical polymer unit to be a measurement of a respective corresponding canonical polymer unit.

A non-canonical polymer unit identified from the analysis can be additionally or alternatively retained as a measurement of a non-canonical polymer unit as being a measurement of a respective corresponding canonical polymer unit.

The non-canonical nucleotides can be introduced into the polynucleotides in place of corresponding canonical bases.

One or more of the polynucleotide strands can comprise four or more different types of non-canonical bases.

The method can further comprise the step of introducing the non-canonical bases into the polynucleotide strands.

The series of measurements can be analysed using a machine learning technique, which has preferably been trained, to attribute a measurement relating to the presence of one or more non-canonical bases in a region of nucleotides to being a measurement of an equivalent region except wherein the one or more types of non-canonical bases have been replaced by respective one or more corresponding canonical bases and wherein the estimation of the consensus sequence is provided wherein the one or more types of non-canonical bases are determined as their corresponding one or more types of canonical base.

Two or more types of non-canonical polymer units can be introduced into one or more of the polynucleotide strands.

Each of the polynucleotides strands can comprise between 30% and 80% non-canonical polymer units.

The series of measurements can be taken during movement of the polymer units with respect to a nanopore.

In some embodiments, measurements of a given type of non-canonical polymer unit are not attributed to a measurement of a respective corresponding canonical polymer unit type.

Thus, in some embodiments, a given non-canonical base type may be base-called. For example, the machine learning technique may be trained to base-call one or more non-canonical bases which frequently occur in vivo, for example 5-methyl-cytosine or 6-methyl-adenine.

As used herein with regard to polymer units, a polymer unit “type” may refer to a given polymer unit chemical species.

In a simplest form, a polymer may comprise multiple polymer units of a single polymer unit type (e.g. “N-N-N-N-N-N”, wherein “N” represents a given polymer unit type). A polymer may comprise polymer units of more than one type, for example at least two types (e.g. “X-Y-X-Y-X-Y”, wherein “X” and “Y” represent different polymer unit types), at least three types (e.g. “X-Y-Z-X-Y-Z”, wherein “X”, “Y” and “Z” represent different polymer unit types), or at least four types (“A-B-C-D-A-B-C-D”, wherein “A”, “B”, “C” and “D” represent different polymer unit types). Polymer units may be present in a polymer in any order and any proportion of polymer unit types.

By way of example, a DNA polynucleotide may typically comprise polymer units (bases) of four different canonical types: A, G, C and T. An RNA polynucleotide may typically comprise polymer units (bases) of four different canonical types: A, G, C and U.

A polymer (e.g. a polynucleotide) may comprise non-canonical polymer units of one or more types. As described herein, in this context a non-canonical polymer unit type may refer to a given non-canonical polymer unit chemical species.

Thus with regard to a polynucleotide, a polymer unit may refer to a nucleotide within the polynucleotide.

By way of example, a polymer (e.g. a polynucleotide) may comprise non-canonical polymer units of at least one, at least two, at least three, or at least four, or more (e.g. at least 1, 2, 3, 4, 5, 6, 7, or 8) types.

A polymer (e.g. when the polymer is a polynucleotide, the polynucleotide) may comprise at least two, at least three, at least four, or more (e.g. at least 2, 3, 4, 5, 6, 7, or 8) types of non-canonical polymer unit (e.g. when the polymer is a polynucleotide, non-canonical base).

Each non-canonical polymer unit type may correspond to a different canonical polymer unit type.

A polymer (e.g. a polynucleotide) may comprise at least two, at least three, or at least four non-canonical polymer unit types, wherein each type of non-canonical polymer unit corresponds to a different canonical polymer unit.

In one embodiment, the polymer is a polynucleotide. In one embodiment, the polynucleotide comprises at least four types of canonical base and at least four types of non-canonical base, wherein each non-canonical base type corresponds to a different canonical base type.

By way of example, a polynucleotide may comprise the canonical base types A, G, C and T (or A, G, C and U), and four non-canonical base types, wherein each non-canonical base type corresponds to a different canonical base type. A polynucleotide may therefore comprise at least eight types of base: at least four types of canonical base and at least four corresponding types of non-canonical base.

A non-canonical polymer unit type may correspond to more than one canonical polymer unit type.

A polymer may comprise more than one non-canonical polymer unit type corresponding to the same canonical polymer unit type.

In one embodiment, a polynucleotide comprises at least two (e.g. at least 2, 3, 4, 5, 6, 7, or 8) types of non-canonical base, wherein at least two of said at least two non-canonical base types correspond to the same canonical base.

In one embodiment, a polynucleotide comprises at least four types of canonical base and at least five types of non-canonical base, wherein at least two of the types of non-canonical base correspond to the same type of canonical base.

The proportion of non-canonical polymer units in a polymer may be varied. By way of example, a polymer may comprise non-canonical polymer units wherein the non-canonical polymer units comprise at least about 10%, at least about 20%, at least about 30%, at least about 40%, at least about 50%, at least about 60%, at least about 70%, at least about 80%, or at least about 90%, of the polymer, when considered as a percentage of the total number of polymer units in the polymer.

The proportion of canonical and corresponding non-canonical polymer unit types in a polymer may be varied, such that for a given polymer unit type at least about 10%, at least about 20%, at least about 30%, at least about 40%, at least about 50%, at least about 60%, at least about 70%, at least about 80%, or at least about 90%, of the instances of said polymer unit type are represented by a corresponding non-canonical polymer unit type.

As described herein, in one aspect of the invention a plurality of polymers is provided.

In one embodiment, the polymers (e.g. polynucleotides) comprise non-canonical polymer units (e.g. non-canonical bases) of at least two, at least three, or at least four types. In one embodiment, each type of non-canonical polymer unit (e.g. non-canonical base) corresponds to a different type of canonical polymer unit (e.g. canonical base).

In one embodiment, the polymers are polynucleotides.

In one embodiment, the polynucleotides comprise the canonical base types A, G, C and T, and at least four different non-canonical base types, wherein each non-canonical base type corresponds to a different canonical base type. Thus, the polynucleotides comprise a non-canonical base corresponding to A, a non-canonical base corresponding to G, a non-canonical base corresponding to C, and a non-canonical base corresponding to T.

In one embodiment, the polynucleotides comprise the canonical base types A, G, C and U, and at least four different non-canonical base types, wherein each non-canonical base type corresponds to a different canonical base type. Thus, the polynucleotides comprise a non-canonical base corresponding to A, a non-canonical base corresponding to G, a non-canonical base corresponding to C, and a non-canonical base corresponding to U.

In one embodiment, the polynucleotides comprise the canonical base types A, G, C and T, and at least five different non-canonical base types (e.g. at least 5, 6, 7, or 8), wherein at least two of said different non-canonical base types correspond to the same canonical base type. Thus, the polynucleotides comprise a non-canonical base corresponding to A, a non-canonical base corresponding to G, a non-canonical base corresponding to C, and a non-canonical base corresponding to T, and further comprise at least one further non-canonical base corresponding to one of A, G, C and T.

In one embodiment, the polynucleotides comprise the canonical base types A, G, C and U, and at least five different non-canonical base types (e.g. at least 5, 6, 7, or 8), wherein at least two of said different non-canonical base types correspond to the same canonical base type. Thus, the polynucleotides comprise a non-canonical base corresponding to A, a non-canonical base corresponding to G, a non-canonical base corresponding to C, and a non-canonical base corresponding to U, and further comprise at least one further non-canonical base corresponding to one of A, G, C and U.

The plurality of polymers (e.g. the plurality of polynucleotides) may be generated by any method known in the art for preparing polymers (e.g. polynucleotides) comprising non-canonical polymer units (e.g. non-canonical bases). By way of example, a plurality of polynucleotides according to the invention may be generated by a method for preparing a polynucleotide comprising non-canonical bases as described herein.

The distribution of the non-canonical polymer units in the polymers is non-deterministic. Thus, the plurality of polymers may comprise polymers in which a proportion (e.g. at least about 10%, 20%, 30%, 40%, 50%, 60%, 70%, 80%, or 90%) of canonical polymer units are substituted with corresponding non-canonical polymer units in a non-deterministic manner.

By way of example, a plurality of polynucleotides may be provided wherein the plurality of polynucleotides has been generated with reference to the target polynucleotide sequence. Each of the polynucleotides comprises a region of nucleotides that corresponds to a region of the target polynucleotide. A proportion of the nucleotide positions in each polynucleotide are substituted with non-canonical bases in a non-deterministic manner. Given the non-deterministic nature of the substitutions, different polynucleotides typically have a different set of nucleotide positions substituted. In some embodiments wherein more than one non-canonical base corresponding to a specific canonical base is present, different strands may have different substitutions at a given nucleotide position. Given the non-deterministic nature of the substitutions, some strands may also have the same position substituted by the same non-canonical base.

Due to the non-deterministic nature of the substitutions, the signal relating to each polynucleotide of the plurality of polynucleotides may be different. One consequence is that any errors present in the analysis of the signal will be non-systematic, thus leading to an improvement in the determination of a consensus sequence.

In embodiments wherein a given non-canonical base type corresponds to more than one canonical base type (for example, wherein a non-canonical base is a universal base), the presence of such a non-canonical base may represent a loss of information in a particular strand with regard to the corresponding canonical base, but because the incorporation of the non-canonical base (for example, universal base) is non-deterministic, a proportion of homologous strands retain the corresponding canonical base and thus enable its identity to be established via consensus.

In yet a further aspect, the invention provides a modified polynucleotide, wherein said modified polynucleotide comprises at least four types of canonical base and at least four corresponding types of non-canonical base, wherein the modified polynucleotide comprises about 40 to about 60% non-canonical bases, optionally about 45 to about 55% non-canonical bases, optionally about 50% non-canonical bases. In yet a further aspect the method provides a method of determining a sequence of a target polymer comprising different types of polymer unit

a. taking a series of measurements of a signal relating to the target polymer

wherein a measurement of the signal is dependent upon a plurality of polymer units and

wherein the polymer units of the target polymer modulate the signal, and wherein the different types of polymer units modulate the signal differently from each other

b. analysing the series of measurements using a machine learning technique that attributes a measurement of one type of polymer unit to be a measurement of a different type of polymer unit;

c. determining the sequence of the target polymer from the analysed series of measurements, wherein the sequence is expressed as a reduced number of different types of polymer units.

The polymer may comprise two or more different types of polymer units, such as four or more different types. The polymer may consist of entirely canonical polymer units, non-canonical polymer units or a combination of canonical or non-canonical units. Measurement of a canonical unit may be attributed to be a measurement of another canonical unit. For example, wherein the polymer is a polynucleotide, the sequence may be expressed as comprising purines and/or pyrimidines. Thus, a measurement of adenine may be attributed as being a measurement of guanine or vice versa. Similarly, measurements of cytosine, thymine and uracil may be expressed as being pyrimidines.

According to a first example of the present invention, there is provided a method of analysis of a series of measurements taken from a polymer comprising a series of polymer units during translocation of the polymer with respect to a nanopore, the method comprising analysing the series of measurements using a machine learning technique and deriving a series of posterior probability matrices corresponding to respective measurements or respective groups of measurements, each posterior probability matrix representing, in respect of different respective historical sequences of polymer units corresponding to measurements prior or subsequent to the respective measurement, posterior probabilities of plural different changes to the respective historical sequence of polymer units giving rise to a new sequence of polymer units.

The series of posterior probability matrices representing posterior probabilities provide improved information about the series of polymer units from which measurements were taken and can be used in several applications. The series of posterior probability matrices may be used to derive a score in respect of at least one reference series of polymer units representing the probability of the series of polymer units of the polymer being the reference series of polymer units. Thus, the series of posterior probability matrices enable several applications, for example as follows.

Many applications involve derivation of an estimate of the series of polymer units from the series of posterior probability matrices. This may be an estimate of the series of polymer units as a whole. This may be done by finding the highest scoring such series from all possible series. For example, this may be performed by estimating the most likely path through the series of posterior probability matrices.

Alternatively, an estimate of the series of polymer units may be found by selecting one of a set of plural reference series of polymer units to which the series of posterior probability matrices are most likely to correspond, for example based on the scores.

Another type of estimate of the series of polymer units may be found by estimating differences between the series of polymer units of the polymer and a reference series of polymer units. This may be done by scoring variations from the reference series.

Alternatively, the estimate may be an estimate of part of the series of polymer units. For example, it may be estimated whether part of the series of polymer units is a reference series of polymer units. This may be done by scoring the reference series against parts of the series of series of posterior probability matrices.

Such a method provides advantages over a comparative method that derives a series of posterior probability vectors representing posterior probabilities of plural different sequences of polymer units. In particular, the series of posterior probability matrices provide additional information to such posterior probability vectors that permits estimation of the series of polymer units in a manner that is more accurate. By way of example, this technique allows better estimation of regions of repetitive sequences, including regions where short sequences of one or more polymer units are repeated. Better estimation of homopolymers is a particular example of an advantage in a repetitive region. In other words, the increase in the complexity or variation in regions in the target polymer, that were repetitive and of low complexity in the original polymer, improves the determination of the sequence.

To gain an intuition why this advantage exists, consider the problem of predicting on which day a parcel will be delivered. The arrival of each parcel is analogous to the extension of a predicted polymer sequence by one unit. A model which predicts states (e.g. Boia et al., DeepNano: Deep Recurrent Neural Networks for Base Calling in Minion Nanopore Reads, Cornell University Website, March 2016) will produce a probability that the parcel is delivered on each future day. If there is a great deal of uncertainty about the delivery date then the probability that the parcel is delivered on any particular day may be less than 50%, in which case the most probable sequence of events according to the model is that the parcel is never delivered. On the other hand, a model which predicts a change with respect to a history state might produce 2 probabilities for each day: 1) the probability that the parcel is delivered if it has not yet been delivered, which will increase as more days pass, and 2) the probability that the parcel is delivered if it has already been delivered, which will always be 0. Unlike the previous model, this model always predicts that the parcel is eventually delivered.

Analogously, state-based models tend to underestimate the lengths of repetitive polymer sequences compared to models that predict changes with respect to a history. This offers a particular advantage for homopolymer sequences because the sequence of measurements produced by a homopolymer tend to be very similar, making it difficult to assign measurements to each additional polymer unit.

Determination of homopolymer regions is particularly challenging in the context of nanopore sequencing involving the translocation of polymer strands, for example polynucleotide strands, through a nanopore in a step-wise fashion, for example by means of an enzyme molecular motor. The current measured during translocation is typically dependent upon multiple nucleotides and can be approximated to a particular number of nucleotides. The polynucleotide strand when translocated under enzyme control typically moves through the nanopore one base at a time. Thus for polynucleotide strands having a homopolymer length longer than the approximated number of nucleotides giving rise to the current signal, it can be difficult to determine the number of polymer units in the homopolymer region. One example of the invention seeks to improve the determination of homopolymer regions.

The machine learning technique may employ a recurrent neural network, which may optionally be a bidirectional recurrent neural network and/or comprise plural layers.

There are various different possibilities for the changes that the posterior probabilities represent, for example as follows.

The changes may include changes that remove a single polymer unit from the beginning or end of the historical sequence of polymer units and add a single polymer unit to the end or beginning of the historical sequence of polymer units.

The changes may include changes that remove two or more polymer units from the beginning or end of the historical sequence of polymer units and add two or more polymer units to the end or beginning of the historical sequence of polymer units.

The changes may include a null change.

The method may employ event calling and apply the machine learning technique to quantities derived from each event. For example, the method may comprise: identifying groups of consecutive measurements in the series of measurements as belonging to a common event; deriving one or more quantities from each identified group of measurements; and operating on the one of more quantities derived from each identified group of measurements using said machine learning technique. The method may operate on windows of said quantities. The method may derive posterior probability matrices that correspond to respective identified groups of measurements, which in general contain a number of measurements that is not known a priori and may be variable, so the relationship between the posterior probability matrices and the measurements depends on the number of measurements in the identified group.

The method may alternatively apply the machine learning technique to the measurements themselves. In this case, the method may derive posterior probability matrices that correspond to respective measurements or respective groups of a predetermined number of measurements, so the relationship between the posterior probability matrices and the measurements is predetermined.

For example, the analysis of the series of measurements may comprise: performing a convolution of consecutive measurements in successive windows of the series of measurements to derive a feature vector in respect of each window; and operating on the feature vectors using said machine learning technique. The windows may be overlapping windows. The convolutions may be performed by operating on the series of measurements using a trained feature detector, for example a convolutional neural network.

According to a second example of the present invention, there is provided a method of analysis of a series of measurements taken from a polymer comprising a series of polymer units during translocation of the polymer with respect to a nanopore, the method comprising analysing the series of measurements using a recurrent neural network that outputs decisions on the identity of successive polymer units of the series of polymer units, wherein the decisions are fed back into the recurrent neural network so as to inform subsequently output decisions.

Compared to a comparative method that derives posterior probability vectors representing posterior probabilities of plural different sequences of polymer units and then estimates the series of polymer units from the posterior probability vectors, the present method provides advantages because it effectively incorporates the estimation into the recurrent neural network. As a result, the present method provides estimates of the identity of successive polymer units that may be more accurate.

The decisions may be fed back into the recurrent neural network unidirectionally.

The recurrent neural network may be a bidirectional recurrent neural network and/or comprise plural layers.

The method may employ event calling and apply the machine learning technique to quantities derived from each event. For example, the method may comprise: identifying groups of consecutive measurements in the series of measurements as belonging to a common event; deriving one or more quantities from each identified group of measurements; and operating on the one or more quantities derived from each identified group of measurements using said recurrent neural network. The method may operate on windows of said quantities. The method may derive decisions on the identity of successive polymer units that correspond to respective identified groups of measurements, which in general contain a number of measurements that is not known a priori and may be variable, so the relationship between the decisions on the identity of successive polymer units and the measurements depends on the number of measurements in the identified group.

The method may alternatively apply the machine learning technique to the measurements themselves. In this case, the method may derive decisions on the identity of successive polymer units that correspond to respective measurements or respective groups of a predetermined number of measurements, so the relationship between the decisions on the identity of successive polymer units and the measurements is predetermined.

For example, the analysis of the series of measurements may comprise: performing a convolution of consecutive measurements in successive windows of the series of measurements to derive a feature vector in respect of each window; and operating on the feature vectors using said machine learning technique. The windows may be overlapping windows. The convolutions may be performed by operating on the series of measurements using a trained feature detector, for example a convolutional neural network.

According to a third example of the present invention, there is provided a method of analysis of a series of measurements taken from a polymer comprising a series of polymer units during translocation of the polymer with respect to a nanopore, the method comprising: performing a convolution of consecutive measurements in successive windows of the series of measurements to derive a feature vector in respect of each window; and operating on the feature vectors using a recurrent neural network to derive information about the series of polymers units.

This method provides advantages over comparative methods that apply event calling and use a recurrent neural network to operate on a quantity or feature vector derived for each event. Specifically, the present method provides higher accuracy, in particular when the series of measurements does not exhibit events that are easily distinguished, for example where the measurements were taken at a relatively high sequencing rate.

The windows may be overlapping windows. The convolutions may be performed by operating on the series of measurements using a trained feature detector, for example a convolutional neural network.

The recurrent neural network may be a bidirectional recurrent neural network and/or may comprise plural layers.

The third example of the present invention may be applied in combination with the first or second examples of the present invention.

The following comments apply to all the examples of the present invention.

The present methods improve the accuracy in a manner which allows analysis to be performed in respect of series of measurements taken at relatively high sequencing rates. For example, the methods may be applied to a series of measurements taken at a rate of at least 10 polymer units per second, preferably 100 polymer units per second, more preferably 500 polymer units per second, or more preferably 1000 polymer units per second.

The nanopore may be a biological pore.

The polymer may be a polynucleotide, in which the polymer units are nucleotides.

The measurements may comprise one or more of: current measurements, impedance measurements, tunneling measurements, FET measurements and optical measurements.

The method may further comprise taking said series of measurements.

The target polymer can be derived from the template or the complement of an original polymer. Said template or complement of the target polymer can have a 3′ or 5′ connection to a polymerase fill-in. The connection can be an adapter. Wherein at least one of the template, complement or polymerase fill-in of the target polymer can comprise canonical and non-canonical polymer units.

The non-canonical bases can be non-deterministically incorporated in to the target polymer.

The polynucleotide can comprise one or more non-canonical bases of one or more different types is generated from its template or complement by use of a polymerase and a proportion of non-canonical bases.

The generated polynucleotide can be covalently attached to the corresponding template or complement via two hairpin adaptors and the resulting construct is circular.

The two hairpin adaptors can be asymmetric.

The polymer can be a polynucleotide. The polymer units can be nucleotide bases and the target polynucleotide can comprise repeat sections of a template polynucleotide strand generated from a circular construct by use of a polymerase and a proportion of non-canonical bases.

The target polynucleotide can comprise repeat alternating sections of a template polynucleotide strand and a complement polynucleotide.

The target polynucleotide can be generated from the circular construct by use of a polymerase and a proportion of non-canonical bases.

The complement can be prepared by at least one of: covalently attaching adaptors to opposite ends of a double stranded polynucleotide; and separating the double stranded polynucleotide to provide complement strands each comprising an adaptor at one end or adaptors at either end.

The method can be synergistically combined with further techniques for improving base calling and/or determining a consensus of a target polymer, or part thereof. The target polymer can be derived from the template or the complement of an original polymer. The template and/or complement of the target polymer can have a 3′ or 5′ connection to a reverse complement thereof. At least one of the template, complement or reverse complement of the target polymer can comprise canonical and non-canonical polymer units. The non-canonical polymer units can be provided by substitution. The non-canonical polymer units can be provided during a polymerase fill-in. The non-canonical bases can be non-deterministically incorporated into the target polymer.

The method, apart from the step of taking the series of measurements, may be performed in a computer apparatus.

According to further examples of the invention, there may be provided an analysis system arranged to perform a method according to any of the first to third examples. Such an analysis system may be implemented in a computer apparatus.

According to yet further examples of the invention, there may be provided such an analysis system in combination with a measurement system arrange to take a series of measurements from a polymer during translocation of the polymer with respect to a nanopore.

In yet another example, a type of measurement system is provided for estimating a target sequence of polymer units in a polymer, such as a nucleic acid. The system uses a polymerase, labelled nucleotides and a detector. Properties of the system depend on detection of the labelled nucleotides as they are incorporated into a copy of the nucleic acid template. By way of example, suitable types of detectors are zero-mode waveguides (Eid et al., 2009 Science) and nanopores (Fuller et al., 2016 PNAS).

Sources of error in single molecule sequencing can occur from the sensing of the same base twice. In sequencing-by-synthesis this can include detecting the label on the nucleotide twice for one incorporation event. If however there is a mix of cognate and non-cognate labelled nucleotides then this source of error can be mitigated against. For example, the sequence of the next nucleotides in the template nucleic acid could be either AC or AAC.

Determining the correct sequence can be difficult due to at least one of the following: (I) In the instance where the true sequence is AC, detecting the label of the T base, being incorporated opposite A, once would result in the correct sequence being determined; (II) In the instance where the true sequence is AC, if the label of the T base is detected twice then this would result in the incorrect sequence being determined, to give an insertion error (AAC); and (III) In the instance where the true sequence was AAC, detecting the labels of two independent T bases being incorporated would result in the correct sequence being determined.

It is therefore not possible to easily determine the sequence as you cannot easily determine whether (II) or (III) has occurred. If, however, the nucleotide pool contains a mix of complementary bases with cognate and non-cognate labels then this source of error can be minimised. For example: (I) In the instance where the true sequence is AC, if the label of the T base is detected twice then this would result in the incorrect sequence being determined, to give an insertion error (AAC); (II) In the instance where the true sequence was AAC, detecting the labels of two different labels from two independent T bases being incorporated would result in the correct sequence being determined; and (III) If you detect T-T* or T*-T then you have a higher certainty that the sequence is AAC. If however, you detect T-T or T*-T* then you can assign a different probability that the sequence is AAC, as it could be AC and you have observed an insertion event. This could then further be used to compare or combine with sequence reads, either inter or intramolecular, to obtain a more accurate consensus.

To allow better understanding, embodiments of the present invention will now be described by way of non-limitative example with reference to the accompanying drawings, in which:

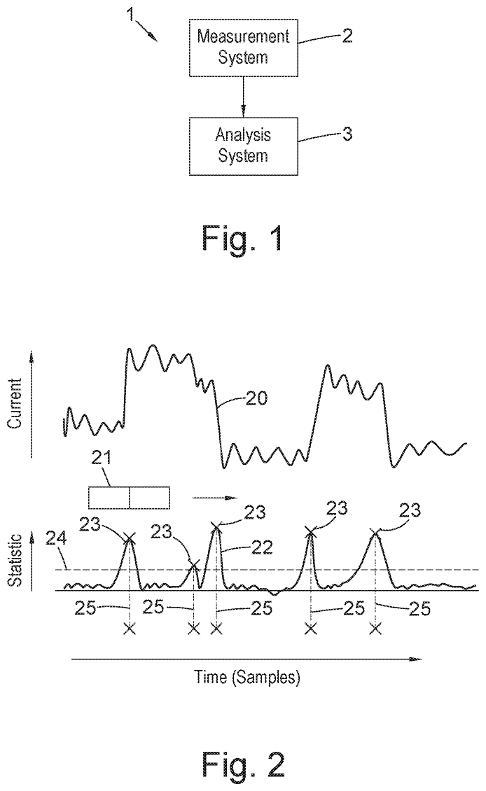

FIG. 1 is a schematic diagram of a nanopore measurement and analysis system;

FIG. 2 is a representation of a segmentation process used to find the boundaries of the events that are input into an analysis system;

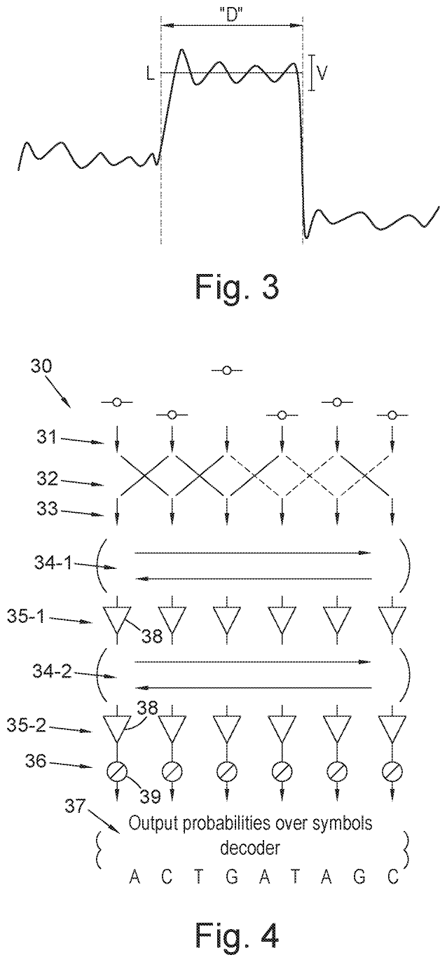

FIG. 3 is a graph of the raw signal illustrating the relationship to example quantities that are summary statistics of an identified event;

[symbol]

FIG. 4 is a schematic diagram illustrating the structure of an analysis system implemented by a recurrent neural net;

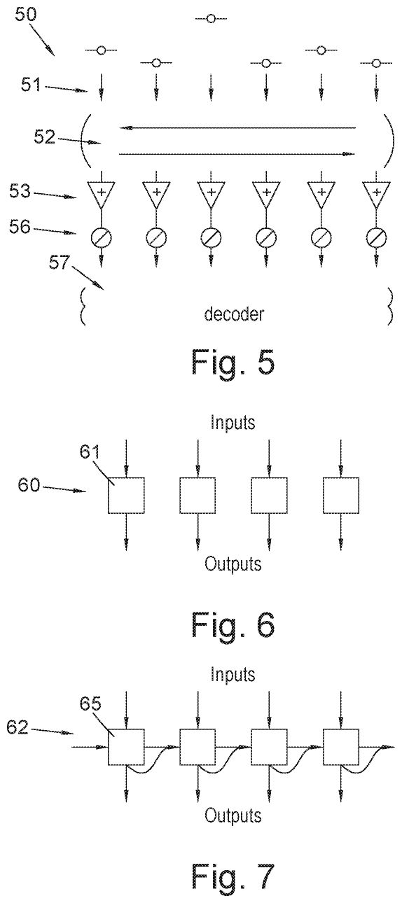

FIG. 5 is a schematic diagram illustrating the structure of a comparative example of an analysis system employing an HMM (Hidden Markov Model) architecture;

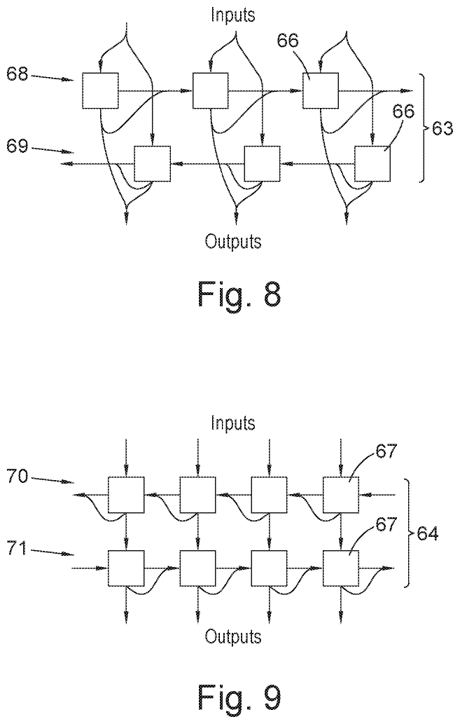

FIGS. 6 to 9 are schematic diagrams of layers in a neural network showing how units of the layer operate on a time-ordered series of input features, FIG. 6 showing a non-recurrent layer, FIG. 7 showing a unidirectional layer, FIG. 8 showing a bidirectional recurrent layer that combines a ‘forward’ and ‘backward’ recurrent layer, and FIG. 9 showing an alternative bidirectional recurrent layer that combines ‘forward’ and ‘backward’ recurrent layer in an alternating fashion;

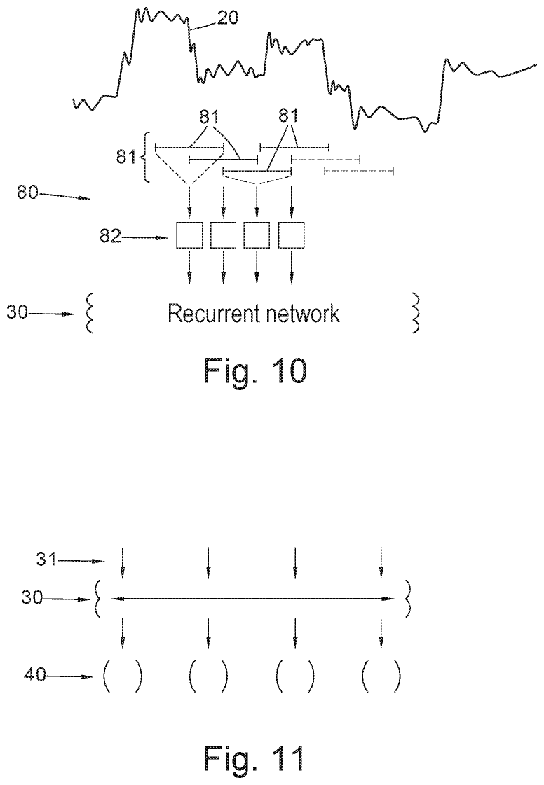

FIG. 10 illustrates a modification to the analysis system of FIG. 4 to operate on measurements (raw data);

FIG. 11 illustrates a modification to the analysis system of FIG. 4;

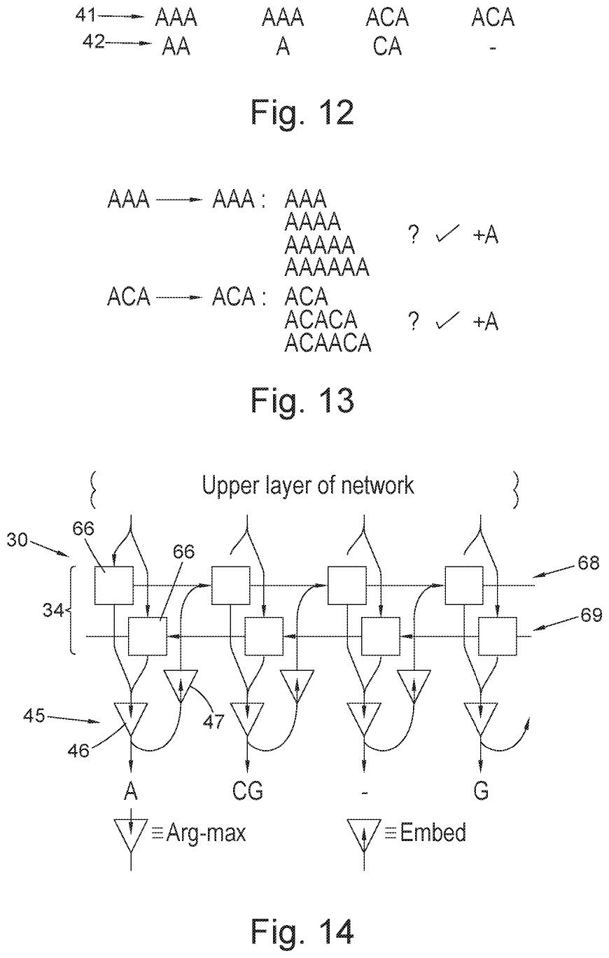

FIG. 12 shows a sample output of the analysis system with the modification of FIG. 11;

FIG. 13 shows some sample cases where the basic method results in an ambiguous estimate of the series of polymer units whereas the sequence fragments of the movement-states in the modification of FIG. 11 are not ambiguous;

FIG. 14 illustrates a modification to the analysis system of FIG. 4 where the decoding has been pushed back into the lowest bidirectional recurrent layer;

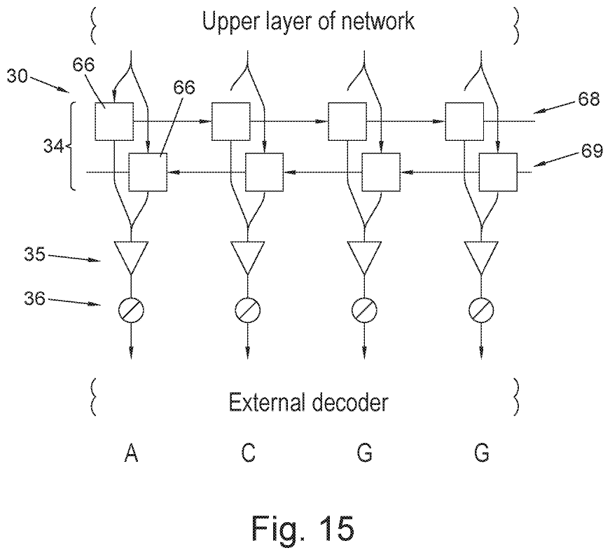

FIG. 15 illustrates, by way of comparison, the final layers of the analysis system of FIG. 4, and its decoder; and

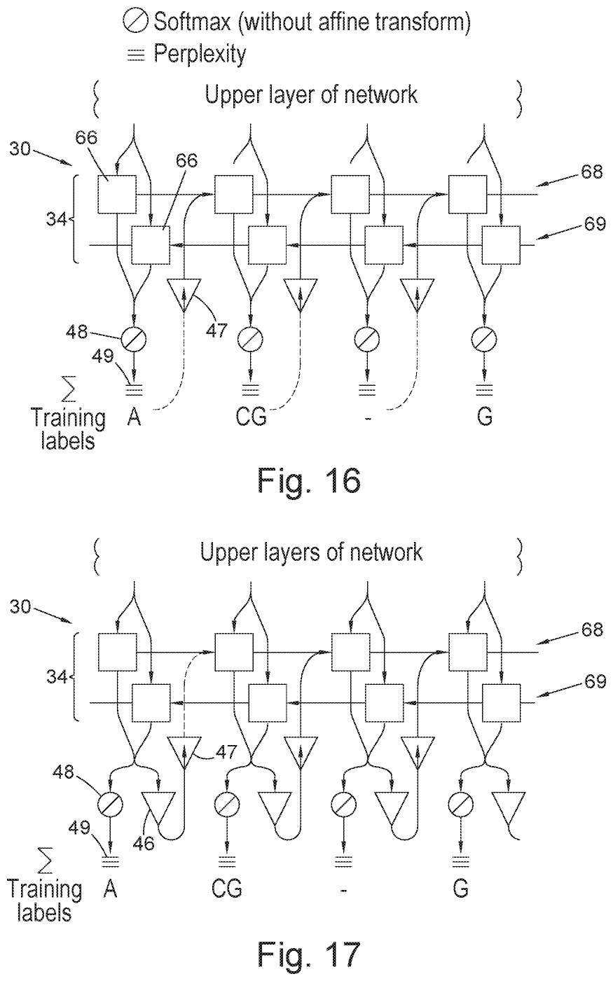

FIGS. 16 and 17 illustrate two alternative modification to the analysis system of FIG. 14 to enable training by perplexity;

FIG. 17 illustrates a modification to the analysis system of FIG. 4 to enable training by perplexity, including arg max units added back into the network so that their output is fed back in;

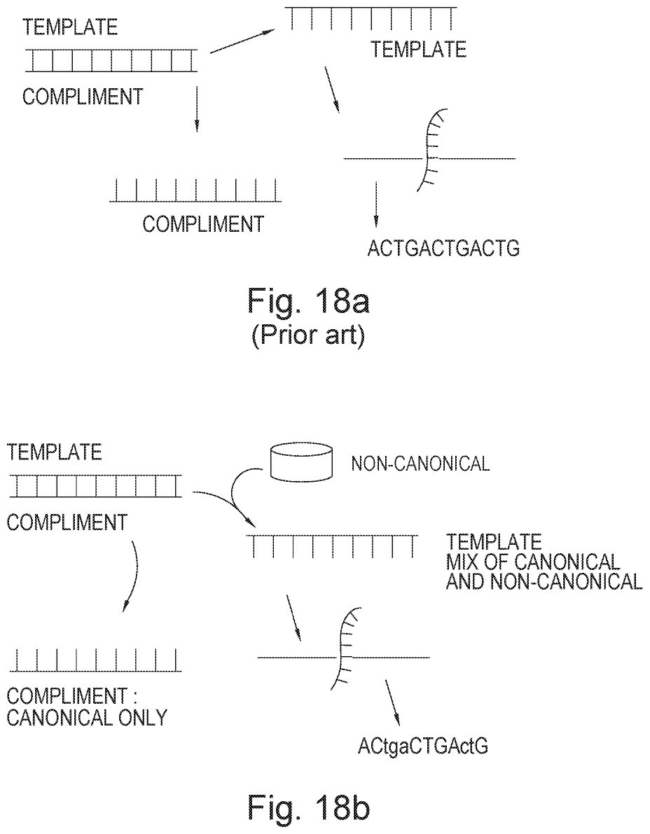

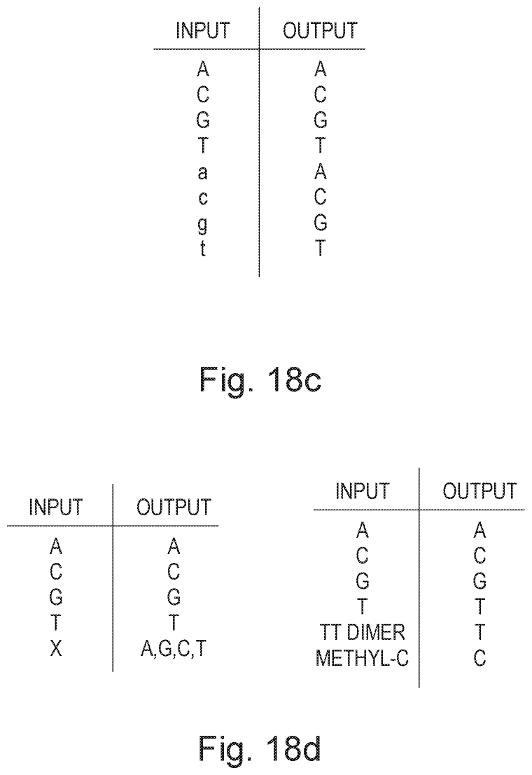

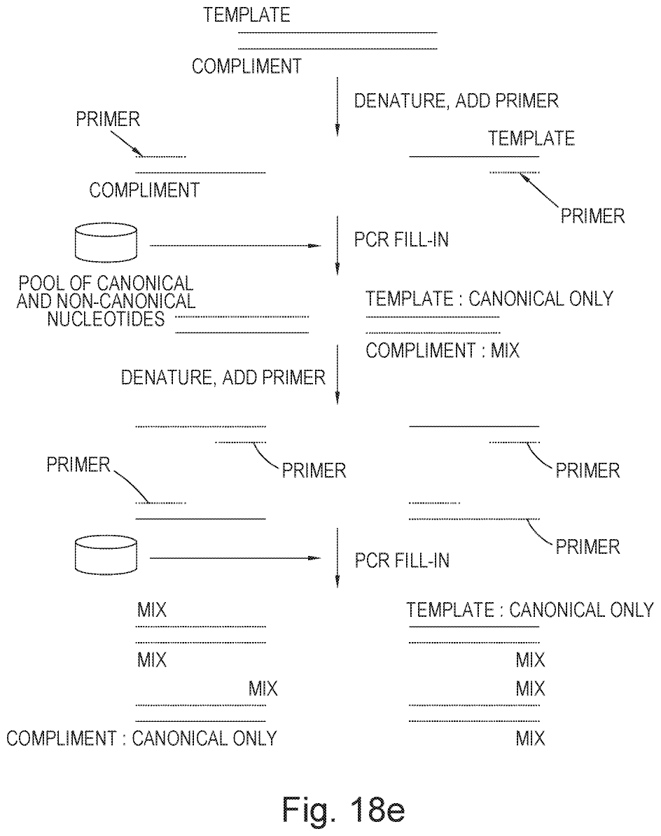

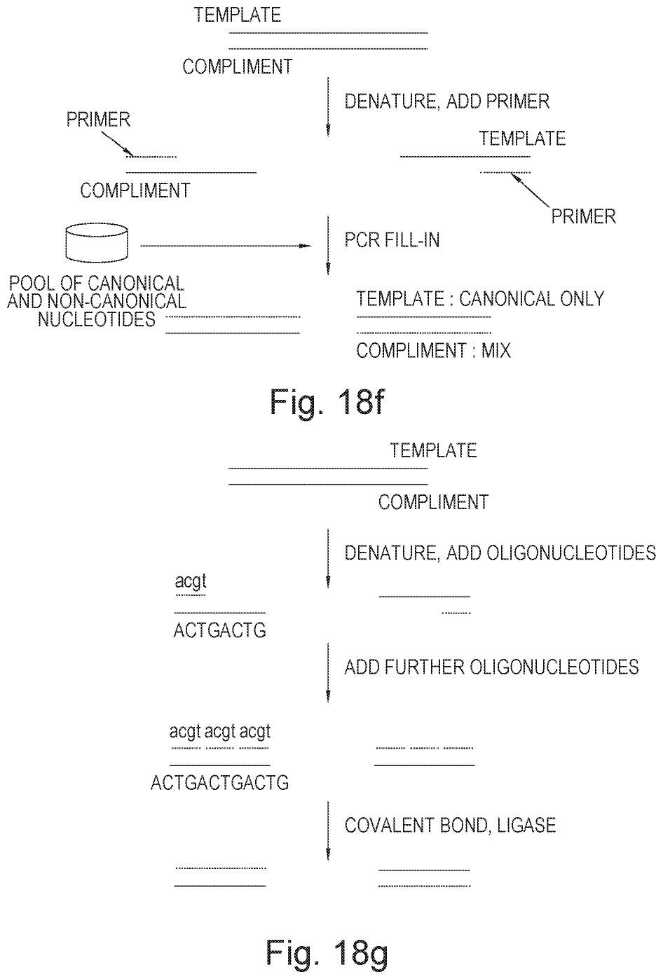

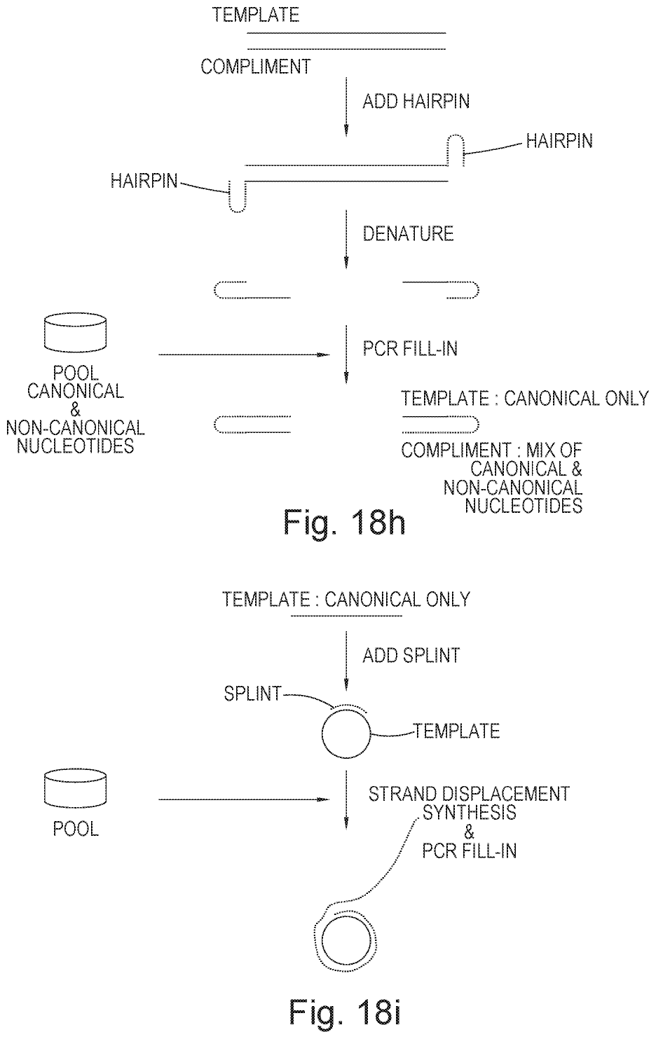

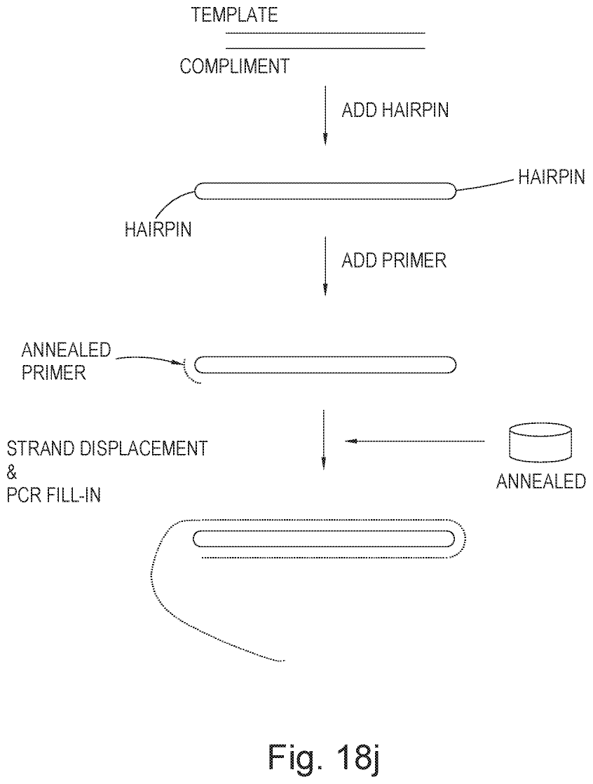

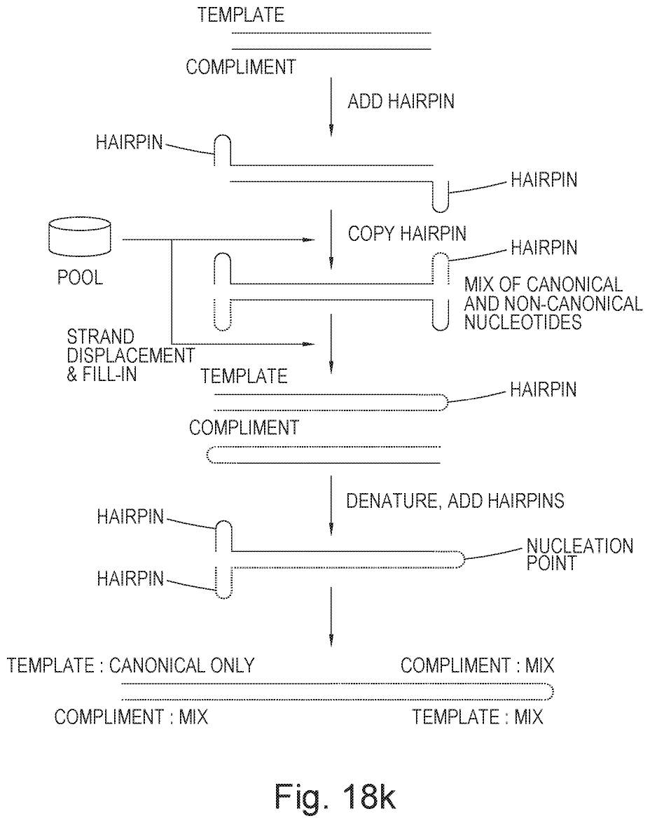

FIG. 18a illustrates a known technique, while FIGS. 18b to 18k illustrate the steps of adding non-canonical bases for analysis and tables indicating the canonical basecall output for a corresponding non-canonical base identified;

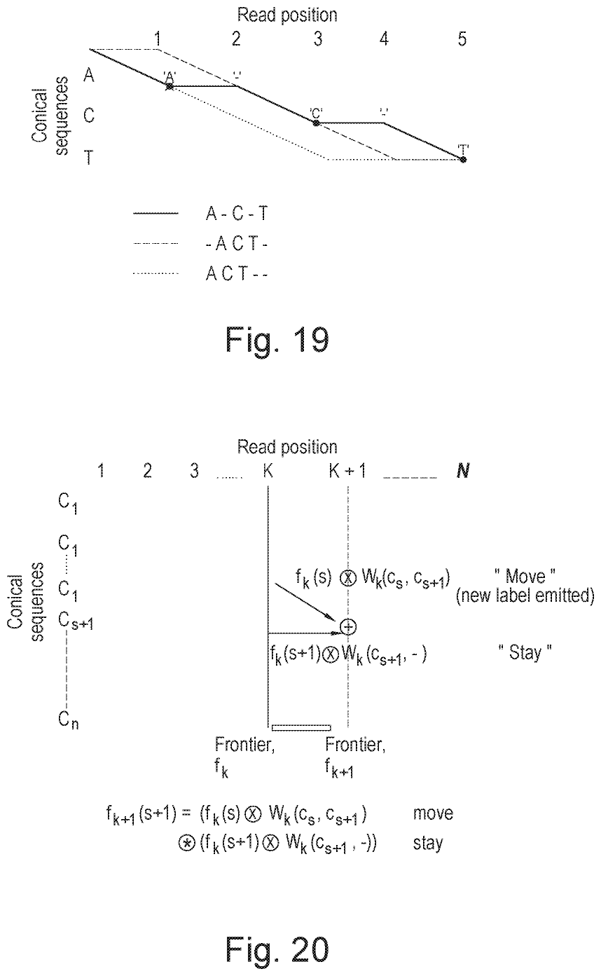

FIG. 19 shows how three possible paths for labelling;

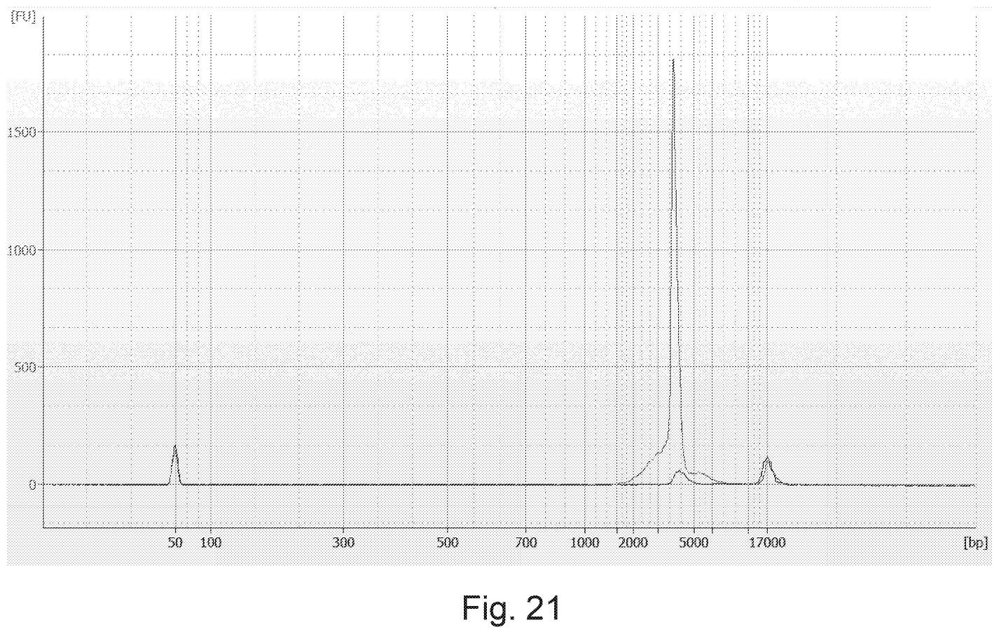

FIG. 20 illustrates the progress of a calculation is shown pictorially in FIG. 2;

FIG. 21 shows an overlay of a 3.6 kb strand subjected to 1× cycle of amplification using 100% dGTAC triphosphates—blue is in the absence of polymerase and red is in presence of polymerase—the presence of the peak in the red trace at 3-4 kb indicates successful amplification; note the absence of a peak here in the blue trace;

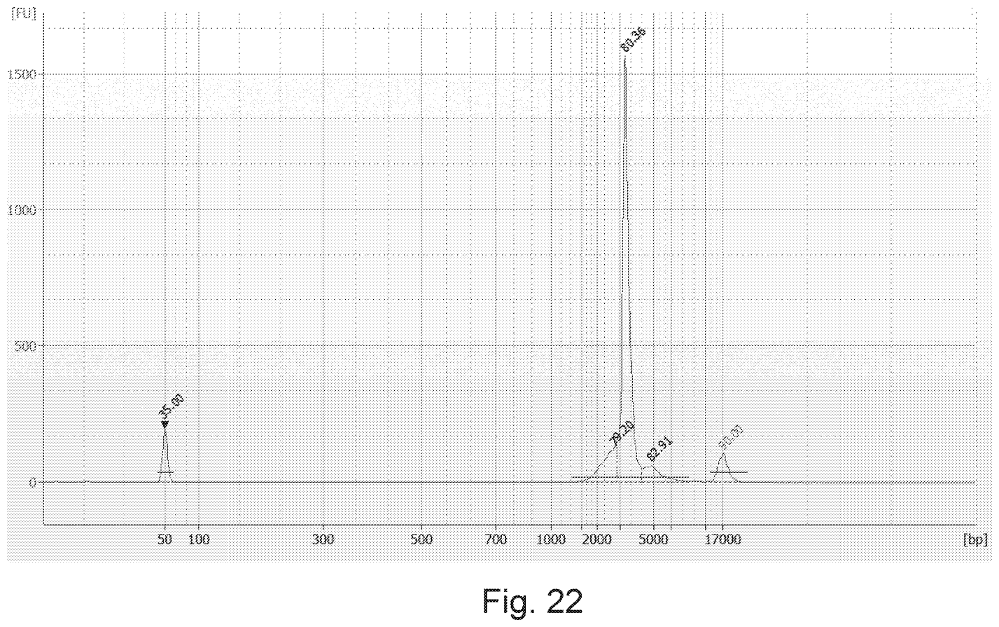

FIG. 22 shows 1× cycle amplification of a 3.6 kb strand using a polymerase and 75% 7-deaza dG, 75% 2-amino dA, 25% dG, 25% dA and 100% dTC triphosphates—the presence of the peak in the red trace at 3-4 kb indicates successful amplification;

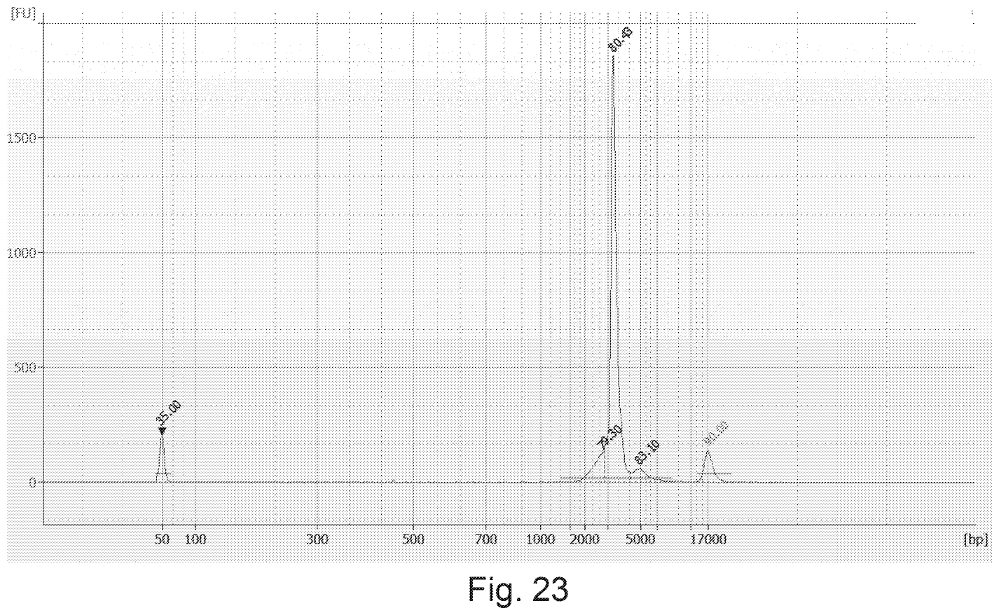

FIG. 23 shows 1× cycle amplification of a 3.6 kb strand using a polymerase and 50% 7-deaza dG, 50% 2-amino dA, 50% dG, 50% dA and 100% dTC triphosphates—the presence of the peak in the red trace at 3-4 kb indicates successful amplification;

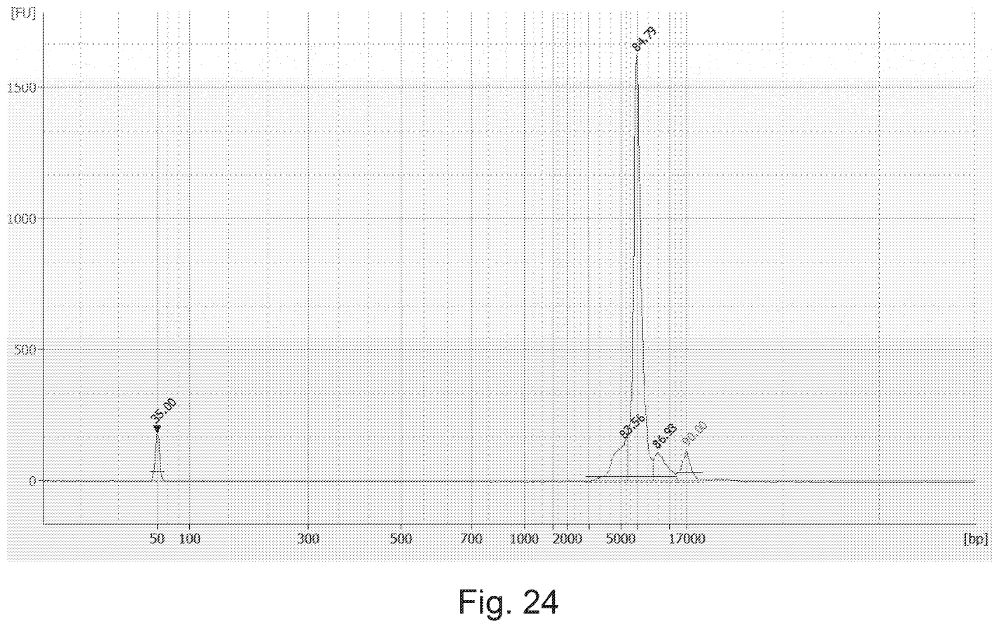

FIG. 24 shows 1× cycle amplification of a 3.6 kb strand using a polymerase and 75% 5-propynyl dU, 75% 5-propynyl dC, 25% dT, 25% dC and 100% dGA triphosphates, wherein the presence of the peak in the red trace at ˜5-6 kb indicates successful amplification—note the presence of the 5-propynyl groups increases the size of the peak, which can be due to the extra size;

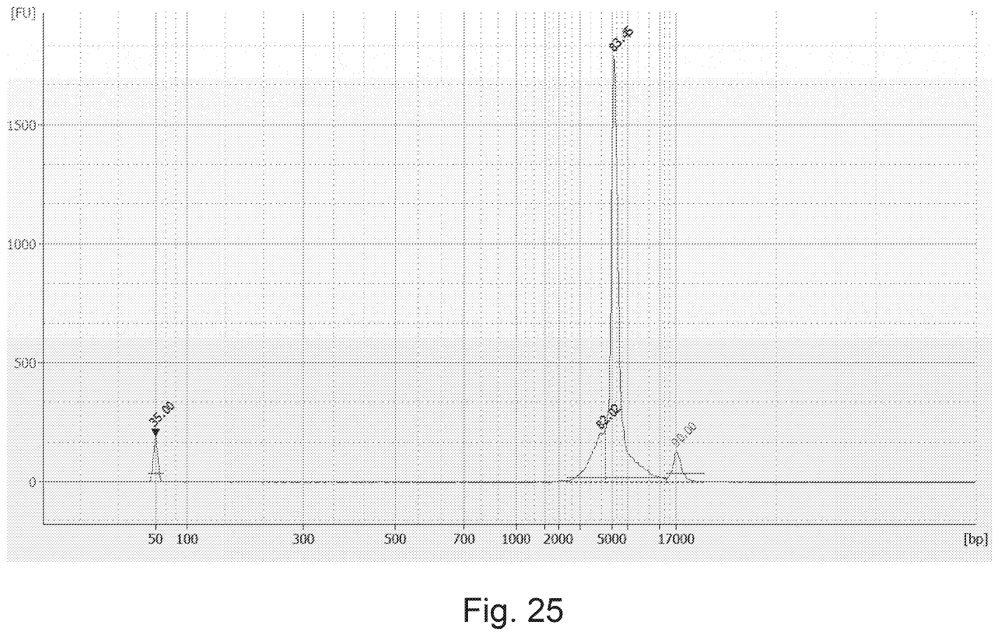

FIG. 25 shows 1× cycle amplification of a 3.6 kb strand using a polymerase and 50% 5-propynyl dU, 50% 5-propynyl dC, 50% dT, 50% dC and 100% dGA triphosphates—the presence of the peak in the red trace at ˜5 kb indicates successful amplification;

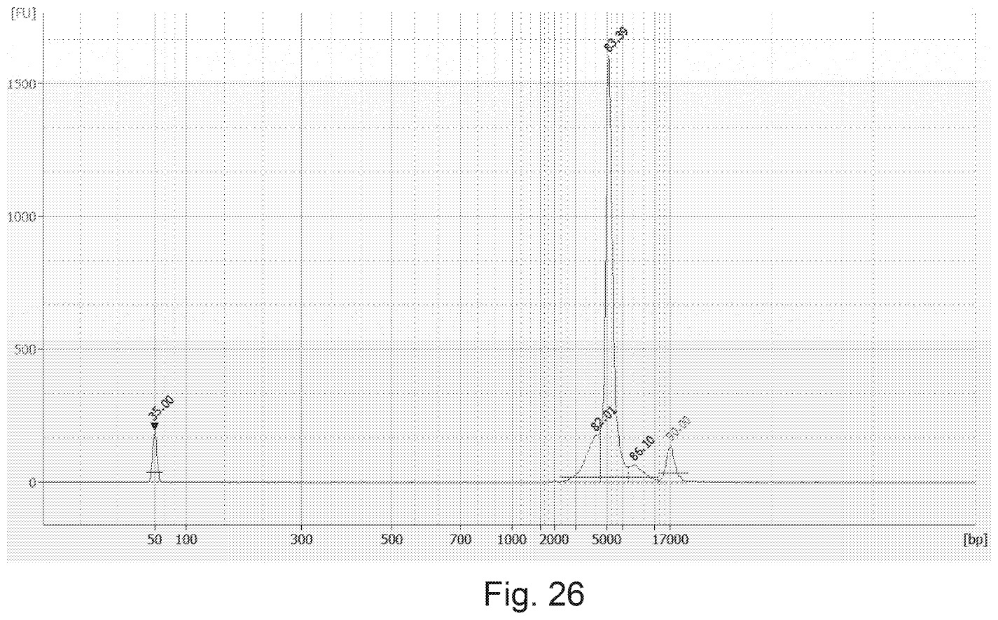

FIG. 26 shows 1× cycle amplification of a 3.6 kb strand using a polymerase and 75% 7-deaza dG, 75% 5-propynyl dU, 75% 2-amino dA, 75% 5-propynyl dC and 25% dGTAC triphosphates—the presence of the peak in the red trace at ˜5-6 kb indicates successful amplification;

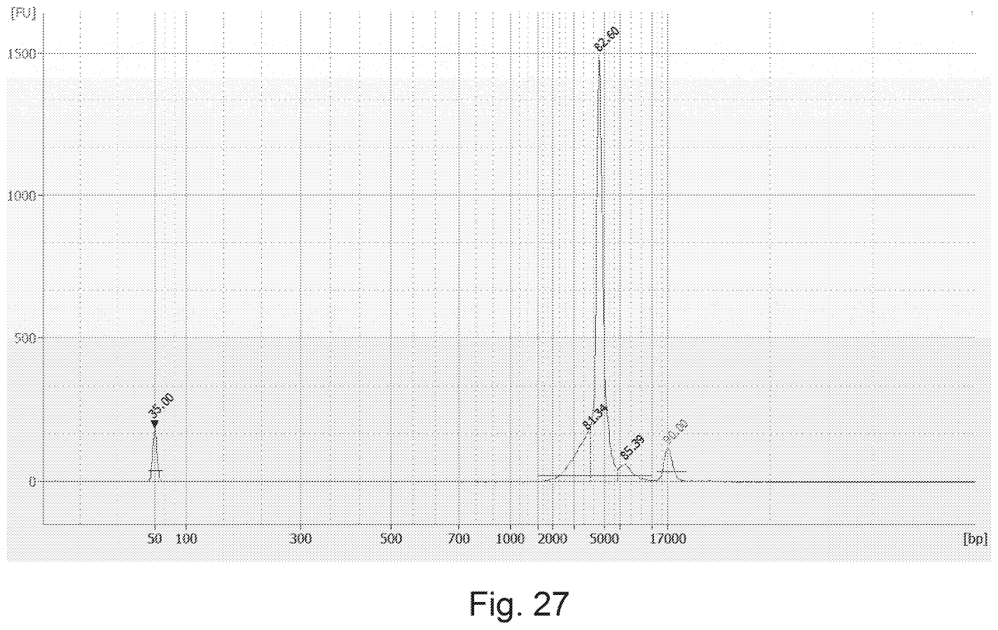

FIG. 27 shows 1× cycle amplification of a 3.6 kb strand using a polymerase and 50% 7-deaza dG, 50% 5-propynyl dU, 50% 2-amino dA, 50% 5-propynyl dC and 50% dGTAC triphosphates—the presence of the peak in the red trace at ˜5 kb indicates successful amplification;

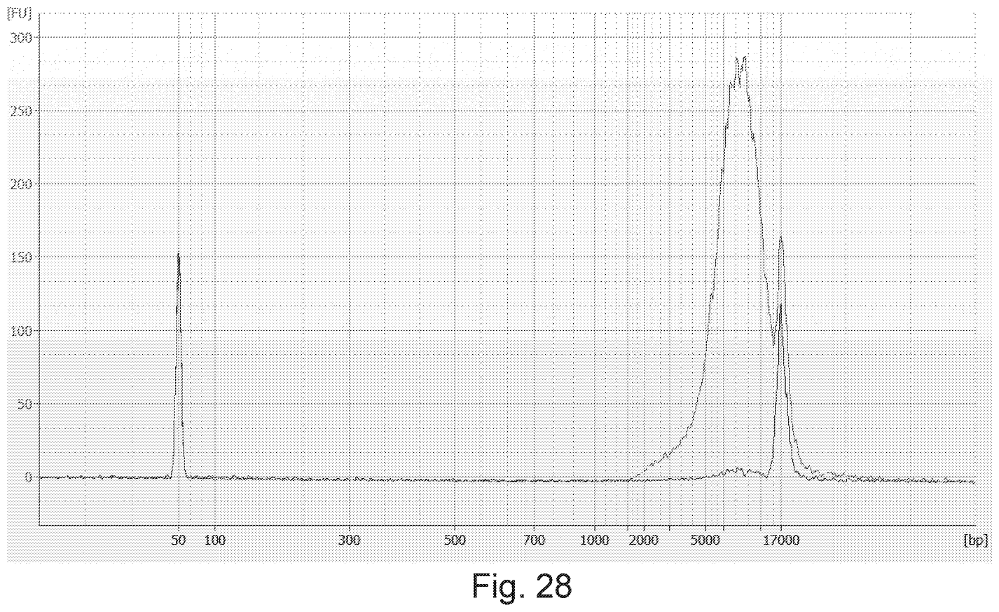

FIG. 28 shows an overlay of the E. coli library subjected to 1× cycle of amplification using 100% dGTAC triphosphates—blue is in the absence of polymerase and red is in presence of polymerase—the presence of the smeared peak in the red trace at 4-10 kb indicates successful amplification; note the absence of a peak here in the blue trace;

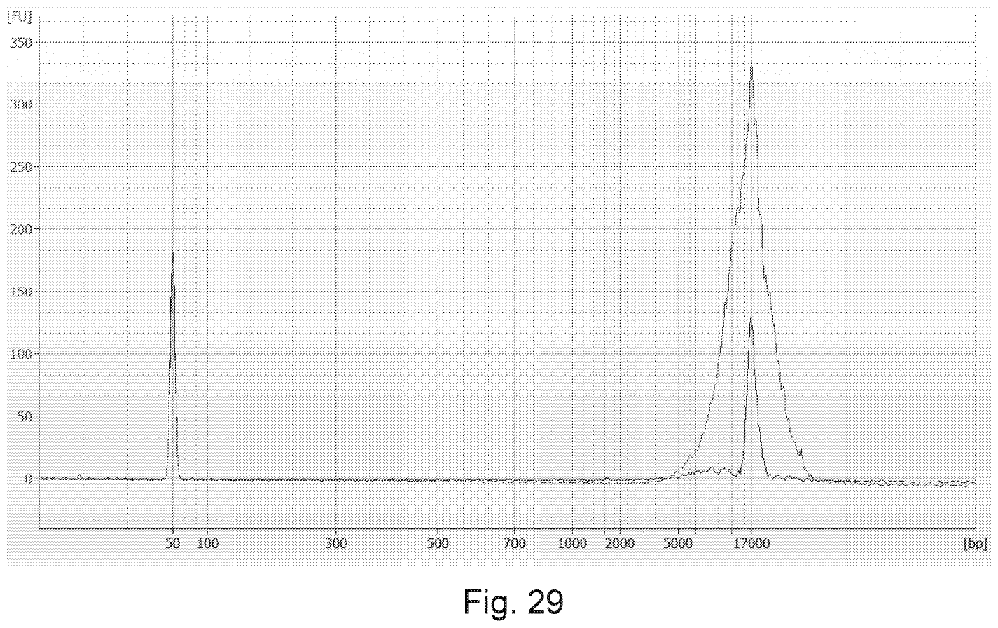

FIG. 29 shows an overlay of the E. coli library subjected to 1× cycle of amplification using 75% 7-deaza dG, 75% 5-propynyl dU, 75% 2-amino dA, 75% 5-propynyl dC and 25% dGTAC triphosphates—blue is in the absence of polymerase and red is in presence of polymerase—the presence of the smeared peak in the red trace at 6-20 kb indicates successful amplification, note the absence of a peak here in the blue trace;

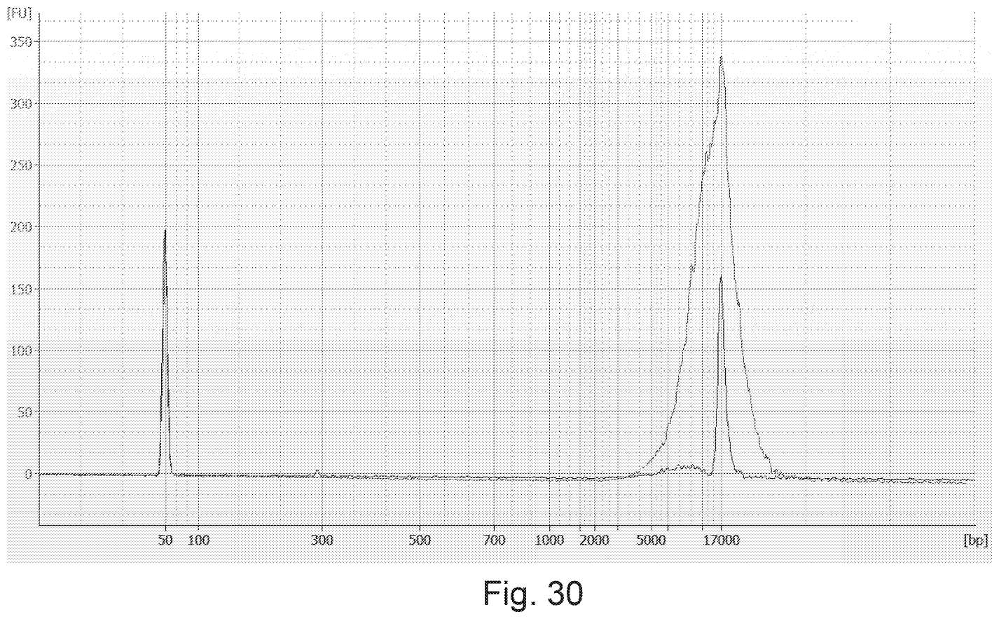

FIG. 30 shows an overlay of the E. coli library subjected to 1× cycle of amplification using 50% 7-deaza dG, 50% 5-propynyl dU, 50% 2-amino dA, 50% 5-propynyl dC and 50% dGTAC triphosphates—blue is in the absence of polymerase and red is in presence of polymerase—the presence of the smeared peak in the red trace at 6-20 kb indicates successful amplification, note the absence of a peak here in the blue trace; and

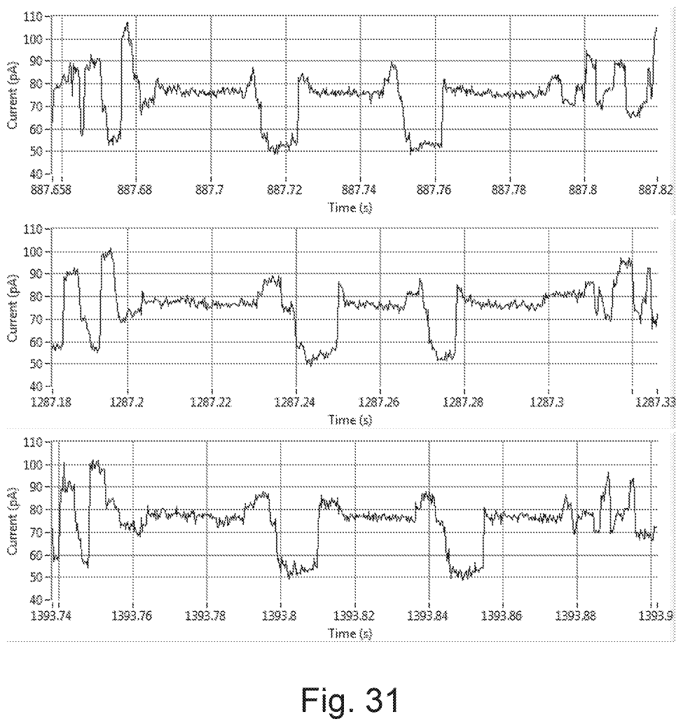

FIG. 31 shows example current traces obtained from the unmodified 3.6 kb products shown in FIG. 21. The central portion of each trace (˜887.69-887.79 secs) corresponds to the sequence TTTTTTTTTTTGGAATTTTTTTTTTGGAATTTTTTTTTT (SEQ ID NO: 4) interacting with the pore. This sequence was designed to give flat homopolymer signal interspersed with two low current level k-mers; and

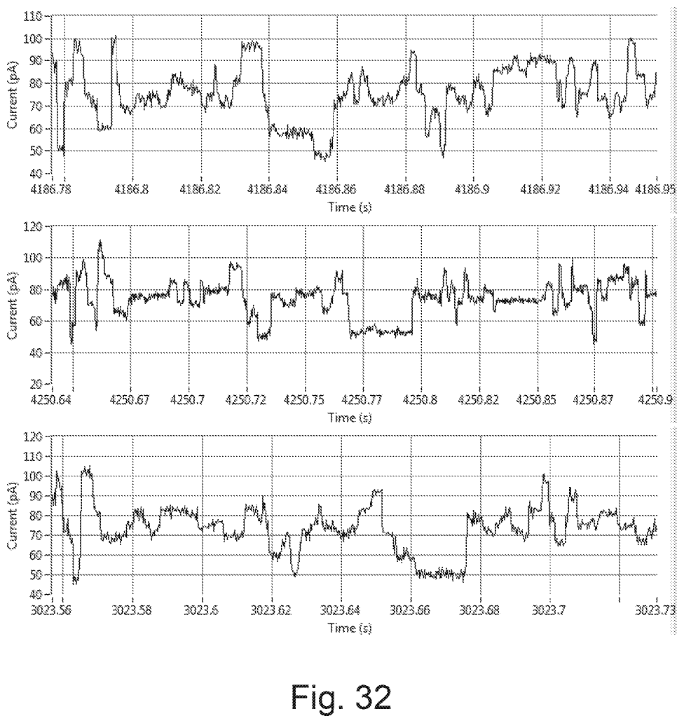

FIG. 32 shows example current traces obtained from the 75% modified base 3.6 kb products shown in FIG. 26. The difference in the current traces, corresponding to the same target sequence, between the above and FIG. 31 can be seen.

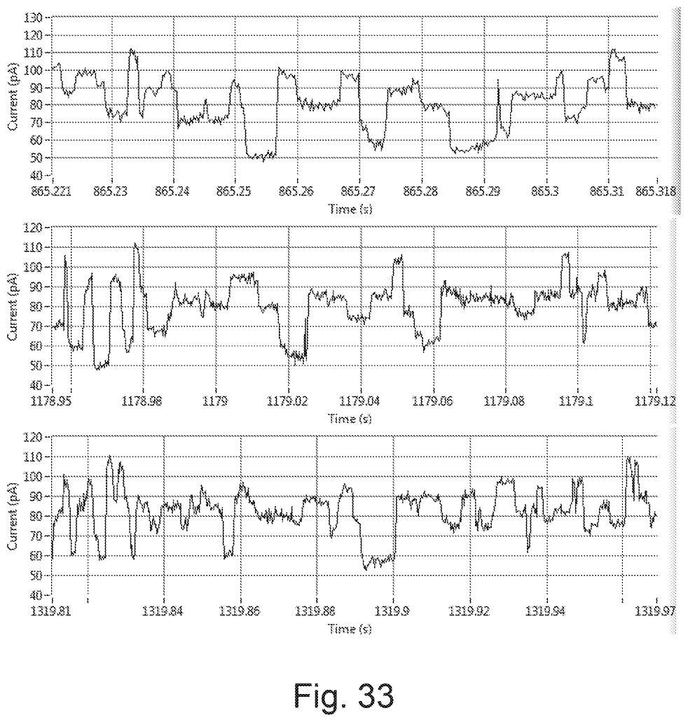

FIG. 33 shows example current traces obtained from the 50% modified base 3.6 kb products shown on FIG. 27. The difference in the current traces, corresponding to the same target sequence, between the above and FIG. 31 can be seen.

FIG. 1 illustrates a nanopore measurement and analysis system 1 comprising a measurement system 2 and an analysis system 3. The measurement system 2 takes a series of measurements from a polymer comprising a series of polymer units during translocation of the polymer with respect to a nanopore. The analysis system 3 performs a method of analysing the series of measurements to obtain further information about the polymer, for example an estimate of the series of polymer units. In general, the polymer may be of any type, for example a polynucleotide (or nucleic acid), a polypeptide such as a protein, or a polysaccharide. The polymer may be natural or synthetic. The polynucleotide may comprise a homopolymer region. The homopolymer region may comprise between 5 and 15 nucleotides.

In the case of a polynucleotide or nucleic acid, the polymer units may be nucleotides. The nucleic acid is typically deoxyribonucleic acid (DNA), ribonucleic acid (RNA), cDNA or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety. The nucleic acid may be single-stranded, be double-stranded or comprise both single-stranded and double-stranded regions. The nucleic acid may comprise one strand of RNA hybridised to one strand of DNA. Typically cDNA, RNA, GNA, TNA or LNA are single stranded.

The polymer units may be any type of nucleotide. The nucleotide can be naturally occurring or artificial. For instance, the method may be used to verify the sequence of a manufactured oligonucleotide. A nucleotide typically contains a nucleobase, a sugar and at least one phosphate group. The nucleobase and sugar form a nucleoside. The nucleobase is typically heterocyclic. Suitable nucleobases include purines and pyrimidines and more specifically adenine (A), guanine (G), thymine (T), uracil (U) and cytosine (C). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate.

A nucleotide may be a basic (i.e. lack a nucleobase). A nucleotide may also lack a nucleobase and a sugar (i.e. is a C3 spacer).

The nucleotides in a polynucleotide may be attached to each other in any manner. The nucleotides are typically attached by their sugar and phosphate groups as in nucleic acids. The nucleotides may be connected via their nucleobases as in pyrimidine dimers.

As used herein, a canonical polymer unit is a polymer unit of a type that is typically found in a particular class of polymer. By way of example, canonical polymer unit types with respect to polynucleotides are typically the nucleobases (and corresponding nucleosides and nucleotides) adenine (A), guanine (G), thymine (T), uracil (U) and cytosine (C).

As used herein, a non-canonical polymer unit is a polymer unit of a type that differs (e.g. has a different molecular structure) from any of the canonical polymer unit types for that class of polymer. By way of example, non-canonical polymer unit types with respect to polynucleotides may be any nucleobases (and corresponding nucleosides and nucleotides) other than A, G, T, U and C as described above.

A non-canonical polymer unit may correspond to a canonical polymer unit. By way of example, a non-canonical polymer unit may be derived from or share structural similarity to a corresponding canonical polymer unit.

In the methods of the invention as described herein polymer units making up a polymer may modulate a signal relating to the polymer. A non-canonical polymer unit may modulate the signal differently from a corresponding polymer unit, thus enabling canonical and non-canonical polymer units to be differentiated.

As used herein, the term “canonical bases” typically refers to the nucleobases adenine (A), guanine (G), thymine (T), uracil (U) and cytosine (C). Canonical bases may form part of canonical nucleosides and canonical nucleotides. Thus, as used herein the term “canonical base” may include canonical nucleosides and canonical nucleotides.

As used herein, the term “non-canonical bases” typically refers to nucleobases that differ from the canonical bases adenine (A), guanine (G), thymine (T), uracil (U) and cytosine (C) as described above. Non-canonical bases may form part of non-canonical nucleosides and non-canonical nucleotides. Thus, as used herein the term “non-canonical base” may include non-canonical nucleosides and non-canonical nucleotides.

A non-canonical base may correspond to a canonical base. By way of example, a given non-canonical base may have substantially the same complementary binding characteristics as a given canonical base, and thus the non-canonical base may be considered as corresponding to the canonical base. The non-canonical base may be derived from, or share structural similarities to, the canonical base such that the non-canonical base has substantially the same complementary binding characteristics as the corresponding canonical base. Thus, a non-canonical base may be a modified canonical base.

A non-canonical base may be capable of specifically hybridising or specifically binding to (i.e. complementing) a canonical base complementary to a canonical base to which the non-canonical base corresponds. By way of example, a non-canonical base corresponding to adenine may be capable of specifically hybridising or specifically binding to thymine. Typically, a non-canonical base hybridises or binds less strongly to those canonical bases that are not complementary to the canonical base to which the non-canonical base corresponds.

A non-canonical base may correspond to more than one canonical base. Thus, a non-canonical base may be capable of specifically hybridising or specifically binding to (i.e. complementing) more than one canonical base. An example of a non-canonical base that corresponds to more than one canonical base is a universal base (e.g. inosine), as described herein.

Many different non-canonical bases are known in the art. A skilled person will be aware of multiple different types of non-canonical bases, wherein “type” may refer to a given non-canonical base chemical species.

Commercially available non-canonical nucleosides include, but are not limited to, 2,6-Diaminopurine-2′-deoxyriboside, 2-Aminopurine-2′-deoxyriboside, 2,6-Diaminopurine-riboside, 2-Aminopurine-riboside, Pseudouridine, Puromycin, 2,6-Diaminopurine-2′-O-methylriboside, 2-Aminopurine-2′-O-methylriboside and Aracytidine. As uracil is not typically found in DNA then in this context 2′-deoxyuridine may be considered as a non-canonical nucleoside.

A non-canonical base may be a universal base or nucleotide. A universal nucleotide is one which will hybridise or bind to some degree to all of the bases in a template polynucleotide. A universal nucleotide is preferably one which will hybridise or bind to some degree to nucleotides comprising the nucleosides adenosine (A), thymine (T), uracil (U), guanine (G) and cytosine (C). A universal nucleotide may hybridise or bind more strongly to some nucleotides than to others. For instance, a universal nucleotide (I) comprising the nucleoside, 2′-deoxyinosine, will show a preferential order of pairing of I-C>I-A>I-G approximately =I-T.

A universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl (C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-0′-methylinosine, 4-nitroindole 2′-deoxyribonucleoside, 4-nitroindole ribonucleoside, 5-nitroindole 2′ deoxyribonucleoside, 5-nitroindole ribonucleoside, 6-nitroindole 2′ deoxyribonucleoside, 6-nitroindole ribonucleoside, 3-nitropyrrole 2′ deoxyribonucleoside, 3-nitropyrrole ribonucleoside, an acyclic sugar analogue of hypoxanthine, nitroimidazole 2′ deoxyribonucleoside, nitroimidazole ribonucleoside, 4-nitropyrazole 2′ deoxyribonucleoside, 4-nitropyrazole ribonucleoside, 4-nitrobenzimidazole 2′ deoxyribonucleoside, 4-nitrobenzimidazole ribonucleoside, 5-nitroindazole 2′ deoxyribonucleoside, 5-nitroindazole ribonucleoside, 4-aminobenzimidazole 2′ deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. A universal nucleotide may comprise 2′-deoxyinosine. A universal nucleotide may be IMP or dIMP. A universal nucleotide may be dPMP (2′-Deoxy-P-nucleoside monophosphate) or dKMP (N6-methoxy-2, 6-diaminopurine monophosphate).

A non-canonical base may comprise a chemical atom or group absent from a related canonical base. The chemical group may be a propynyl group, a thio group, an oxo group, a methyl group, a hydroxymethyl group, a formyl group, a carboxy group, a carbonyl group, a benzyl group, a propargyl group or a propargylamine group. The chemical group or atom may be or may comprise a fluorescent molecule, biotin, digoxigenin, DNP (dinitrophenol), a photo-labile group, an alkyne, DBCO, azide, free amino group, a redox dye, a mercury atom or a selenium atom.

Commercially available non-canonical nucleosides comprising chemical groups which are absent from canonical nucleosides include, but are not limited to, 6-Thio-2′-deoxyguanosine, 7-Deaza-2′-deoxyadenosine, 7-Deaza-2′-deoxyguanosine, 7-Deaza-2′-deoxyxanthosine, 7-Deaza-8-aza-2′-deoxyadenosine, 8-5′(5'S)-Cyclo-2′-deoxyadenosine, 8-Amino-2′-deoxyadenosine, 8-Amino-2′-deoxyguanosine, 8-Deuterated-2′-deoxyguanosine, 8-Oxo-2′-deoxyadenosine, 8-Oxo-2′-deoxyguanosine, Etheno-2′-deoxyadenosine, N6-Methyl-2′-deoxyadenosine, 06-Methyl-2′-deoxyguanosine, 06-Phenyl-2′deoxyinosine, 2′-Deoxypseudouridine, 2-Thiothymidine, 4-Thio-2′-deoxyuridine, 4-Thiothymidine, 5′ Aminothymidine, 5-(1-Pyrenylethynyl)-2′-deoxyuridine, 5-(C2-EDTA)-2′-deoxyuridine, 5-(Carboxy)vinyl-2′-deoxyuridine, 5,6-Dihydro-2′-deoxyuridine, 5,6-Dihydrothymidine, 5-Bromo-2′-deoxycytidine, 5-Bromo-2′-deoxyuridine, 5-Carboxy-2′-deoxycytidine, 5-Fluoro-2′-deoxyuridine, 5-Formyl-2′-deoxycytidine, 5-Hydroxy-2′-deoxycytidine, 5-Hydroxy-2′-deoxyuridine, 5-Hydroxymethyl-2′-deoxycytidine, 5-Hydroxymethyl-2′-deoxyuridine, 5-Iodo-2′-deoxycytidine, 5-Iodo-2′-deoxyuridine, 5-Methyl-2′-deoxycytidine, 5-Methyl-2′-deoxyisocytidine, 5-Propynyl-2′-deoxycytidine, 5-Propynyl-2′-deoxyuridine, 6-O-(TMP)-5-F-2′-deoxyuridine, C4-(1,2,4-Triazol-1-yl)-2′-deoxyuridine, C8-Alkyne-thymidine, dT-Ferrocene, N4-Ethyl-2′-deoxycytidine, 04-Methylthymidine, Pyrrolo-2′-deoxycytidine, Thymidine Glycol, 4-Thiouridine, 5-Methylcytidine, 5-Methyluridine, Pyrrolocytidine, 3-Deaza-5-Aza-2′-O-methylcytidine, 5-Fluoro-2′-O-Methyluridine, 5-Fluoro-4-O-TMP-2′-O-Methyluridine, 5-Methyl-2′-O-Methylcytidine, 5-Methyl-2′-O-Methylthymidine, 2′,3′-Dideoxyadenosine, 2′,3′-Dideoxycytidine, 2′,3′-Dideoxyguanosine, 2′,3′-Dideoxythymidine, 3′-Deoxyadenosine, 3′-Deoxycytidine, 3′-Deoxyguanosine, 3′-Deoxythymidine and 5′-O-Methylthymidine.

A non-canonical base may lack a chemical group or atom present in a related canonical base.

A non-canonical base may have an altered electronegativity compared with a related canonical base. The non-canonical base having an altered electronegativity may comprise a halogen atom. The halogen atom may be attached to any position on the non-canonical base, nucleoside or nucleotide, such as the nucleobase and/or the sugar. The halogen atom is preferably fluorine (F), chlorine (Cl), bromine (Br) or iodine (I). The halogen atom is most preferably F or I.

Commercially available non-canonical nucleosides comprising a halogen include, but are not limited to, 8-Bromo-2′-deoxyadenosine, 8-Bromo-2′-deoxyguanosine, 5-Bromouridine, 5-Iodouridine, 5-Bromouridine, 5-Iodouridine, 5′-Iodothymidine and 5-Bromo-2′-O-methyluridine.

A non-canonical base may be naturally-occurring or non-naturally-occurring.

Naturally-occurring non-canonical bases may be found in polynucleotides in vivo. An example of a naturally-occurring non-canonical base is a naturally-occurring methylated base, e.g. 5-methyl-cytosine or 6-methyl-adenine.

Multiple methods are known in the art for preparing polynucleotides comprising non-canonical bases.

By way of example, a polynucleotide comprising one or more non-canonical bases may be prepared by contacting a template polynucleotide with a polymerase under conditions in which the polymerase forms a modified polynucleotide using the template polynucleotide as a template. Examples of suitable polymerases include Klenow or 9o North. Such conditions are known in the art. For instance, the polynucleotide is typically contacted with the polymerase in commercially available polymerase buffer, such as buffer from New England Biolabs®. The temperature is preferably from 20 to 37° C. for Klenow or from 60 to 75° C. for 9o North. A primer or a 3′ hairpin is typically used as the nucleation point for polymerase extension. Hairpins are known from WO2013/014451, which is incorporated herein by reference in its entirety.

The template polynucleotide may be contacted with a population of free nucleotides. The polymerase uses the free nucleotides to form the modified polynucleotide based on the template polynucleotide. The identities of the free nucleotides in the population determine the composition of the modified polynucleotide. Each free nucleotide in the population is capable of hybridising or binding to one or more of the nucleotide species in the template polynucleotide. Each free nucleotide in the population is typically capable of specifically hybridising or specifically binding to (i.e. complementing) one or more of the nucleotide species in the template polynucleotide. A nucleotide specifically hybridises or specifically binds to (i.e. complements) a nucleotide in the template polynucleotide if it hybridises or binds more strongly to the nucleotide than to the other nucleotides in the template nucleotide. This allows the polymerase to use complementarity (i.e. base pairing) to form the modified polynucleotide using the template polynucleotide. Typically, each free nucleotide specifically hybridises or specifically binds to (i.e. complements) one of the nucleotides in the template polynucleotide.

By way of further example, a polynucleotide comprising one or more non-canonical bases may be prepared by contacting a template polynucleotide with a ligase under conditions in which the polymerase forms a modified polynucleotide using the template polynucleotide as a template. Examples of suitable ligases include Taq or E. coli and T4. Such conditions are known in the art. For instance, the polynucleotide is typically contacted with the ligase in commercially available polymerase buffer, such as buffer from New England Biolabs™. The temperature is preferably from 12 to 37° C. for E. coli and T4 or from 45 to 75° C. for Taq. A primer or a 3′ hairpin is typically used as the nucleation point for ligation extension.

The template polynucleotide may be contacted with a population of free oligonucleotides. The ligase uses the free oligonucleotides to form the modified polynucleotide based on the template polynucleotide. The identities of the free oligonucleotides in the population determine the composition of the modified polynucleotide. Each free oligonucleotide in the population is capable of hybridising or binding to four or more of the nucleotide species in the template polynucleotide. Each free nucleotide in the population is typically capable of specifically hybridising or specifically binding to (i.e. complementing) four or more of the nucleotide species in the template polynucleotide. A nucleotide specifically hybridises or specifically binds to (i.e. complements) nucleotides in the template polynucleotide if it hybridises or binds more strongly to the nucleotides than to the other nucleotides in the template nucleotide. This allows the ligase to use complementarity (i.e. base pairing) to form the modified polynucleotide using the template polynucleotide. Typically, each free oligonucleotide specifically hybridises or specifically binds to (i.e. complements) six of the nucleotides in the template polynucleotide”

A template polynucleotide may be a target polynucleotide. A template polynucleotide may be a complement of a target polynucleotide. A template polynucleotide may correspond in part or in whole to a target polynucleotide. A template polynucleotide may be a complement of a part or the whole of a target polynucleotide.

In some embodiments, a polynucleotide comprising one or more non-canonical bases may be prepared by enzymatic conversion of one or more canonical bases to a corresponding non-canonical base. By way of example, a polynucleotide comprising canonical bases may be contacted with an enzyme capable of converting one or more types of canonical base to a corresponding non-canonical base type. Examples of such enzymes include DNA- and RNA-methyltransferase enzymes. In some embodiments, a polynucleotide comprising one or more non-canonical bases may be prepared by chemical conversion of one or more canonical bases to a corresponding non-canonical base. By way of example, a polynucleotide comprising canonical bases may be contacted with a chemical capable of converting one or more types of canonical base to a corresponding non-canonical base type. Examples of such chemicals include formic acid, hydrazine, dimethyl sulphate, Osmium tetroxide and some vanadate compounds”

A non-canonical base may also comprise a pyrimidine dimer, for example a thymine dimer. Such a dimer may be introduced into a polynucleotide by the action of ultraviolet light. The products of template dependent synthesis can also be modified. The products can be formed using a population of canonical bases and then the product modified to contain non-canonical bases. The products can be formed using a population of canonical and non-canonical bases and then the product further modified to contain more of the same or different non-canonical bases.

The accuracy of nanopore sequencing can be improved by analysing polymers, or strands, comprising canonical and non-canonical polymer units. The polymers used in the analysis are referred to as target polymers or target strands. These target polymers are derived from an original polymer or strand that has a common canonical sequence, either by origin or design. This original polymer can be referred to as a homologous strand. To be clear, the original polymer originates from a sample to be analysed, such as swab from the inside of a cheek of a human.

The original polymer is copied many times and non-canonical polymer units are added to these copies to create target polymers. The measurement signal is obtainable by passing a target polymer through a sequencing device, such as those produced by Oxford Nanopore Technologies, and can process the signal read or processed from the device to provide a sequence. The estimate of the sequence can provide a basecall.

The analysis of the measurements to determine the sequence can use machine learning, as described below.

The creation of target polymers from an original polymer or strand that has a common canonical sequence, is achievable by substituting one or more of the canonical bases i.e. A, C, G and T, with alternative bases, which can be non-canonical. These alternative bases, when passed through a nanopore, produce a different signal compared to the corresponding canonical base. The alternative bases of the target polymer are provided and subsequently located in a non-deterministic manner.

Alternative bases with non-specific binding can be used. The alternative bases can contain modifications, fluorophore groups or atoms with a distinct nuclear magnetic resonance for example, that allow measurements, such as orthogonal measurements, of their presence and location to be made. Additionally, or alternatively, rather than substitution of a canonical base with an alternative base, other alterations to the polymer could be made to produce similar effects to those described. For example, deliberately inducing the formation of pyrimidine dimers via exposure to UV light, or as a further example, excision of the nucleobase to leave the only backbone.

The level of substitution of the bases can be at proportions of between about 1% and about 99%, but preferably between about 30% and about 70%, but preferably still about 50%. The proportion of the substitution can be approximately the same for each substituted base and/or the type of substitution. The proportion of the substitution can be different for each substituted base and/or the type of substitution.

As a result of the non-deterministic nature of the substitution, different target polymers or target strands have alternative bases, such as non-canonical bases, located at different positions with respect to the original base in the original polymer that has been copied to be analysed.

By providing a plurality of alternative bases for a given canonical base, then different target polymers can have different substitutions at a given position. In light of the non-deterministic nature of the substitutions, some target polymers will have the same position substituted by the same alternative, i.e. the sets of positions for different strands are not mutually exclusive.

Determining a sequence of a target polymer comprising polymer units by taking a series of measurements of a signal relating to the target polymer, which can be derived from passing the alternative polymer strand through a nanopore, involves a measurement of the signal that is dependent upon a plurality of polymer units.

The target polymer modulates the signal, and accuracy is improved because the non-canonical polymer units in the target polymer modulate the signal differently from a corresponding canonical polymer unit. To illustrate this difference, the signal of a target polymer derived from the bases CcAGT is different from the otherwise identical bases in the original polymer that has the bases CCAGT. With the alternative bases substituted for canonical bases the signal measured is picking up or identifying the alternative or non-canonical units. By way of example, an alternative base ‘c’ is substituted for canonical base ‘C’. By way of another example, a canonical base can be replaced with inosine, which does not correspond to any one of the bases C, A, G or T but is recognised as such and the subsequent analysis can attribute this non-canonical base as ‘non-canonical’ or any one of A, C, G or T.

The signal is processed using analysis methods that are aware of the alternative bases. The analysis methods comprise a base calling method, a consensus method, and any ancillary processing required to derive the result.

A preferred example of a base calling method is where the base calling method has been trained to attribute the influence of the alternative bases on the signal, to the canonical bases.

Upon sequencing multiple target polymers or strands, it will be appreciated that the signal is modulated in different ways for different strands, by the set of substitutions being different in different strands. While the presence of many alternative bases may make the individual base calls less accurate, it will also be appreciated that any base calling errors will be less systematic and that the consensus sequence will be more accurate as a result.

The method can also be applied when the alternative bases used have non-specific binding. Non-specific represents a loss of information in each strand about the canonical sequence but, because the incorporation of alternative bases is non-deterministic, some proportion of homologous strands retain the canonical base and so its identity can be established by consensus.

While alternative bases in the target polymer can produce a series of measurement that can be analysed to recognise these alternative bases they can be analysed, preferably using a machine learning technique, to attribute a measurement of an alternative base, such as non-canonical polymer unit, to be a measurement of a respective corresponding canonical polymer unit.

Because of the non-deterministic incorporation of canonical and alternative bases into the target polymer, the underlying sequence of bases is not known and will vary on a strand-to-strand basis even if said strands are copies of the same original polymer or template or are biological replicates of the same region of a genome. Even though each strand contains alternative bases, there is still an associated canonical sequence—what would it have been if no alternative bases were present in the sample preparation—and it is of interest to call this directly rather than attempting to infer the type and location of any alternatives. In other words, despite there being 5 or more bases in the target polymer the analysis only attributes canonical values to the signal such that the determined sequence consists of bases from the group of A, C, G and T.

The machine learning technique is preferably trained and uses a model. A trained machine learning technique can be used to estimate the canonical sequence from one or more reads. Before such a technique is applied, it must be trained on a representative set of reads with associated canonical sequences. How such a set can be obtained is described below, we now describe how training may be performed given the unique features of this problem.

The method can use machine learning methods involving the likes of Neural Networks, Recurrent Neural Networks, Random Forests or Support Vector Machines, which are often trained in a supervised fashion, where the training set consists of an explicit relationship or registration between the input signal and the output labels. The input signal is derived from the target polymer, which includes a mixture of canonical and alternative bases. The output labels, or identity of the bases, that the machine learning method attributes to the sequence can be a mixture of canonical and alternative bases or only canonical bases.

An output having a mixture of bases can provide a detailed set of data for the purposes of the subsequent alignment of sequenced target polymers and the formation of the consensus

Consensus methods are well known in the art and can be readily applied. In cases where the base caller attributes the influence of non-canonical bases to canonical bases, the resulting base call comprises a canonical sequence and methods can be applied with little modification. In cases where non-canonical bases are present in the base call, the consensus method can be modified such that non-canonical bases are aligned to their canonical partner. In cases where a non-specific non-canonical base is used, the consensus method can be modified such that the non-specific non-canonical base aligns non-specifically. Such alignments may be achieved, for example, by using a custom substitution matrix or scoring system.

However, such a detailed set of data can increase the computational resource or cost required to align the sequence of the target polymer and form the consensus. Therefore, analysing the measurements to output only canonical bases has the effect of (i) consolidating the detailed measurements using a machine learning technique, which improves the accuracy and/or (ii) simplifying the alignment and formation of the consensus because the process is based on only the four canonical bases, albeit four bases that have been accurately determined because the target polymer comprised a mixture of canonical and alternative polymer units.

FIGS. 18a to 18k support, by way of example, the explanation of the integration of the non-canonical bases in the target polymer to be read.

FIG. 18a represents what is known, for reference. A double-stranded DNA molecule comprising only canonical polymer units is divided such that one of the template or complement of the original polymer is passed through a nanopore to identify the individual polymer units of the original polymer. In FIG. 18a the template is passed through the pore. The template can be basecalled. Further templates can be basecalled and the basecalls can be aligned and used to determine a consensus.

FIG. 18b is an example of the invention in which a double-stranded DNA molecule, which is the original polymer, is denatured and amplified such that substitutions are made and canonical bases are substituted with non-canonical bases, from a supply of non-canonical bases, to produce a target polymer. The substitutions are non-deterministic. In the example of FIG. 18b, the template of the original polymer is subjected to substitutions such that the target polymer has four canonical bases A, C, G and T and four corresponding non-canonical bases a, c, g and t i.e. a mix of canonical and non-canonical bases. After passing through the pore the base caller can call only the canonical bases i.e. four (4) bases from eight (8), or a variation thereof. The way in which raw signal from the pore is processed can vary. The template having a mix of canonical and non-canonical bases becomes the target polymer, which can be basecalled. Further templates can become further target polymers and those can be basecalled too. The basecalls can be aligned and used to determine a consensus.

The way in which the method utilises the presence of the stochastically distributed non-canonical bases can vary. In the examples provided herein the target polymers are basecalled. Additionally or alternatively the raw signals received from a pore after passing a template polymer therethrough can be used to determine the sequence of the target polymer, such raw signal analysis using techniques disclosed in WO13/041878 herein incorporated by reference in its entirety. Overall, however, the computational efficiency can be improved by finally base calling or determining a consensus having only canonical bases and/or the systematic errors can be reduced by the stochastic distribution of non-canonical bases.

FIG. 18c is a table showing the ‘input’ identified by a basecaller, which includes canonical and non-canonical bases identifiable from the target polymer. The corresponding ‘output’ is consolidated to canonical bases. The consolidation of the input to a canonical-only output can occur at a an individual basecall level. The consolidation of the input to a canonical-only output can also be performed in the determination of the consensus from a plurality of basecalls that contain a mixture of canonical and non-canonical units. When a consensus is formed, non-canonical bases can be aligned to their canonical partner. Through the non-deterministic location of non-canonical bases and the subsequent consolidation the systematic errors can be reduced.

In FIG. 18d, by way of example, two alternative input-output tables are shown. They illustrate that a base caller can attribute the influence of a non-canonical bases to one or more canonical bases. Examples include: a non-specific non-canonical base “X” being identified as any canonical base; a methylated “C′” being identified as a canonical “C”; and a “TT dimer” being identified as a canonical “T”. The tables herein are for illustrative purposes only and the consolidation can be implemented using custom substitution matrices or scoring systems.

While the final output from a base call or consensus determination is the identification of canonical bases the intermediate processing can use the raw signal read from a sensor analysing the target polymer. Each of the canonical and non-canonical inputs will influence the raw signal generate in their own way. It can be beneficial for machine learning techniques to analyse the raw signal in order to determine the output—at basecall and/or consensus level.

The invention can be synergistically applied to known techniques for improving base calling and determining consensus. By way of example, the target polymer can have first region and a second region that are reverse compliments of each other—this template and complement can be connected with a hairpin. The target polymer can be derived from the template or the complement of an original polymer, wherein said template or complement of the target polymer has a 3′ or 5′ connection (adapter) to a corresponding reverse complement that is formed using a polymerase fill-in.

The substitutions made to produce a target polymer, as described in relation to FIG. 18b, can be applied in various ways to a template, a complement and/or a reverse complement connected via a hairpin connection.

In FIGS. 18e and 18f the solid lines denote an original portion of a double-stranded DNA molecule i.e. a template or a complement derived therefrom, being parts of the original polymer. The stages in FIGS. 18 e and 18f are carried out using polymerases and nucleotides. A short dotted line indicates a primer, while a longer dotted lines indicates the primer combined with the extension product from the polymerase.

FIG. 18e illustrates 5 stages, with 4 transitions (indicated by downward arrows), that demonstrate how modified polynucleotides can be prepared via amplification, such as polymerase chain reaction (PCR). The method includes a polymerase, a template nucleic acid and a pool of canonical and non-canonical nucleotides. These are cycled according to standard PCR techniques.

The first stage of FIG. 18e begins with a double-stranded DNA molecule, which is denatured and a primer added to produce, at the second stage, a separate template and complement, each having a respective primer attached at one end, and each comprising only canonical bases. The produce of the second stage is then subjected to a polymerase fill-in, said fill-in using a pool, said pool containing canonical and non-canonical nucleotides or bases. The second stage is transformed to produce at the third stage (i) a template having only canonical bases connected, via a primer, to a complement having a mix of canonical and non-canonical bases, and (ii) a complement having only canonical bases connected, via a primer, to a template having a mix of canonical and non-canonical bases.

The produce at the third stage is denatured and a primer added to produce, at the fourth stage, four units each having a primer attached. These four units are (i) a template having a mix of nucleotides or bases, (ii) a template having only canonical bases, (iii) a complement having a mix of bases, and (iv) a complement template having only canonical bases. The produce of the fourth stage, that is each unit of the fourth stage, is subjected to a polymerase fill-in, said fill-in using a pool of canonical and non-canonical nucleotides. This produces, at the fifth stage, (i) a template having a mix of bases connected via a primer to a complement having a mix of bases, (ii) a template having only canonical bases connected via a primer to a complement having a mix of bases, (iii) a complement having a mix of bases connected via a primer to a template having a mix of bases, and (iv) a complement template having only canonical bases connected via a primer to a template having a mix of bases. The cycle of denaturing, adding primers and filling-in can be repeated.

FIG. 18f has the first three stages of FIG. 18e. Modified polynucleotides as target polymers wherein one strand is the original strand, consisting of canonical nucleotides, and the other strand is a synthesis product, consisting of a mixture of canonical and non-canonical nucleotides. Having one strand having only canonical units and another, derived therefrom i.e. the complement or reverse complement, allows the determination of the bases to include a comparison between original canonical bases and stochastically positioned non-canonical bases.

Alternatively, the synthesis can be carried out using a ligase and random oligonucleotides hybridised to the target nucleic acid template. This alternative is shown in FIG. 18g having 4 stages, with 3 transitions that demonstrate how modified ligation and oligonucleotides can be used to create a target polymer for analysis. The first stage of FIG. 18g begins with a double-stranded DNA molecule, which is denatured and oligonucleotides are added. In FIG. 18g solid lines denote an original portion of a double-stranded DNA molecule, which is the original polymer—just one is shown in the second stage as “acgt”. A short dotted line indicates the oligonucleotide. Between stages two and three further oligonucleotides are added. By stage four, the oligonucleotides are covalently bonded by a ligase. The oligonucleotides can consist of non-canonical bases or a mixture of canonical and non-canonical bases.

Further alternatively, synthesis can occur using a hairpin—3′ hairpin added to the 3′ end of template nucleic acids via a number of techniques, such as adapter ligation or incorporation into a 5′ primer. In FIG. 18h there are 4 stages shown, with 3 transitions that demonstrate how a hairpin can be used to initiate synthesis. Hairpins are indicated by hook-shaped lines, which in the second stage are short dotted lines because they comprise a mix of canonical and non-canonical bases—they and function as primers. The first stage of FIG. 18h begins with a double-stranded DNA molecule, and hairpins are added to the end of the template and the complement. In FIG. 18h solid lines denote an original portion of a double-stranded DNA molecule, which is the original polymer. Between stages 2 and 3 the DNA molecule is denatured to produce a separate original template and original complement, each with a hairpin. The produce of the third stage, that is each unit of the third stage, is subjected to a polymerase fill-in, said fill-in using a pool, said pool comprising a mixture of canonical and non-canonical nucleotides.

Either extension from a hairpin, or adding a hairpin to the product of a primer initiated synthesis reaction, allows for information from the original template nucleic acid to be compared or combined with the synthesis product strand.

Concatemers of synthesised products containing canonical and non-canonical nucleotides can also be prepared. This can be performed with either single or double stranded DNA as the starting template nucleic acid. The three most common techniques of concatemer formation are shown, by way of example, in FIGS. 18i, 18j and 18k.

In FIG. 18i, the first stage begins with a template having only canonical polymer units. Its end is then connected via a ligase. A splint, which functions as a primer, is added. Using strand displacement synthesis and a polymerase fill-in using a pool of canonical and non-canonical nucleotides, a reverse complement is created repeatedly. This reverse complement has a mix of nucleotides. This reverse compliment can be analysed directly during its creation. Alternatively, this reverse complement can be analysed after its creation. By way of example, it can be analysed by passing it through a nanopore.

In FIG. 18j the first of 4 stages begins with a double-stranded DNA molecule. Hairpins are added to connect the ends of the template and complement. An annealed primer is added to the second stage and, thereafter, a strand displacement polymerase creates a strand of repeats of the template and complement, said strand being filled-in using a pool of canonical and non-canonical nucleotides. This strand can be analysed directly during its creation. Alternatively, this strand can be analysed after its creation. By way of example, the strand can be analysed by passing it through a nanopore.

In FIG. 18k, the first of 6 stages begins with a double-stranded DNA molecule. One hairpin is added to the template and one hairpin is added to the complement, although the ends of the molecule are not connected. Between the second and third stages the hairpins are copied, and the copies comprise a mix of canonical and non-canonical nucleotides. Then, the double-stranded DNA molecule is denatured and the original template and complement—having only canonical bases—are filled-in using a pool of canonical and non-canonical nucleotides. A further nucleation point and hairpin is added between stages 4 and 5, wherein a PCT fill-in occurs. The produce at stage 5 is subjected to a subsequent fill-in to produce a target polymer having a strand having a first portion (template) having only canonical units and then a sequences of alternating complements and templates, said repeating sequence having a mix of canonical and non-canonical nucleotides as illustrated.

In each of the examples of 18b to 18k the presence of non-canonical units in the target polymer increases the levels of complexity or variation in the signals derived therefrom. This can increase the levels of complexity of variation in all areas of the target polymer. In particular, the range of signals derived from repetitive regions of the original polymer, such as homopolymer regions, is increased in corresponding areas of the target polymer.

For rolling-linear amplification the original template nucleic acid is incorporated into the sequencing product. This provides the ability to compare a strand containing only canonical bases with a series of products that contain a mixture of canonical and non-canonical bases.

The output of all of the methods above can be analysed using techniques including de novo sequencing, sequencing using a reference genome, 1-dimensional sequencing in which the compliment follows the template through the pore or 2-dimensional sequencing.

By way of example, the preparation of the target polymer can use various methods, such as those techniques disclosed in: U.S. Pat. No. 6,087,099; WO2015/124935; or PCT/GB2019/051314—all of which are herein incorporated by reference in their entirety.

All of the methods herein can, additionally or alternatively, be used to create a strand of nucleotides having only canonical bases, which can then be modified either enzymatically or chemically after the synthesis reaction in order to provide the mix of canonical and non-canonical bases in the target polymer.

Due to the non-deterministic nature of the PCR fill-in, or oligonucleotide matching, the signal relating to each polynucleotide of the plurality of polynucleotides may be different. One consequence is that any errors present in the analysis of the signal will be non-systematic, thus leading to an improvement in the determination of a consensus sequence.

Because of the non-deterministic incorporation of canonical and alternative bases into the target polymer, the underlying sequence of bases is not known and will vary on a strand-to-strand basis even if said strands are copies of the same original polymer or template or are biological replicates of the same region of a genome. Even though each strand contains alternative bases, there is still an associated canonical sequence—what would it have been if no alternative bases were present in the sample preparation—and it is of interest to call this directly rather than attempting to infer the type and location of any alternatives. In other words, despite there being 5 or more bases in the target polymer the analysis only attributes canonical values to the signal such that the determined sequence consists of bases from the group of A, C, G and T.

The above methods are provided, by way of example, to demonstrate the preparation of a target polymer to be sequenced—the target polymer having canonical and non-canonical polymer units. During the analysis of the measurements made of the target polymer—typically using a machine learning technique—the method attributes a measurement of a non-canonical polymer unit to being a measurement of a respective corresponding canonical polymer unit. This attribution can be applied at the base calling level and/or during the formation of the consensus. The sequence of the target polymer can then be determined from the analysed series of measurements.

In the preparation of the target polymer, which is derived from the template or the complement of an original polymer, a connection is made to, for example, a PCR fill-in or ligated oligonucleotide. In the target polymer at least one of the template, complement or fill-in comprises canonical and non-canonical polymer units. Non-canonical bases are non-deterministically incorporated into the target polymer.

While the examples herein can be applied to the analysis of all of the target polymer the analysis can, additionally or alternatively, be selectively applied to specific regions of the target polymer. By way of example, the determination of the sequence of the target polymer can focus on specific regions having at least one of (i) particular intervals of signal determined to be of interest, (ii) particular intervals corresponding to regions of the polymer identified as being of interest e.g. a homopolymer, (iii) a simple repetitive pattern of polymer units, and (iv) regions with a particularly biased composition of polymer units.

The determination of sequence can be performed in more than one stage. By way of a non-restrictive example, the determination can focus on the identification of a repeat unit then number of repeats.

The determination of sequence—for either the complete target polymer, or part thereof—can be performed by considering a plurality of series of measurements, each identified as having being from target polymers with the same canonical sequence in the region of interest. The identification can be performed using techniques like those described in WO13/121224, herein incorporated by reference in its entirety. Identification can be performed by making an initial determination of the sequence of polymer units for each series of measurements.

Analysing the series of measurements of a target polymer using a machine learning technique can require training, which requires taking in to account training a base caller in the field of machine learning that accommodates (i) the incomplete knowledge of ground truth sequence for each strand, and (ii) the unknown registration between input signal and output labels.

The incomplete knowledge of ground truth sequence for each strand is a consequence of the non-deterministic presence and location of alternative bases that are formed in the target polymer when it is synthesised from the original polymer. Even in the case where two strands are synthesised complements from the same original molecule, they will still differ in their pattern of canonical and alternative bases and there is no ‘ground truth’ sequence to use when training. To address the differences between target polymers in training the machine learning technique is trained against the canonical sequence i.e. the original polymer from which the target polymer was synthesised. The sequence of canonical bases in the common template strand i.e. the original polymer, allows a base calling method to be trained and still produce a useful output that can be used in the same applications as traditional DNA sequencing techniques.

Issues associated with the unknown registration between input signal and output labels can be referred to as “registration-free”, and such registration-free methods of training can offer benefits over a conventional labelling strategy because the exact mapping of signal to sequence is not required to be specified. Without using a registration-free approach to training, an estimate of registration between the signal and labels must be obtained and this registration is then assumed to be correct despite the presence of mistakes; such mistakes would then trained into the machining learning approach and lead to a loss of base calling accuracy.

Obtaining an estimate of the registration can involve assuming that the registration proceeds in a regular fashion, or by agreement with labels produced by previously obtained model that is been constrained to call the correct sequence of labels. Further, such estimates could be further constrained using additional knowledge about the system like distinctive patterns of signal or other markers.

Rather than training a model from an estimate of the registration, with its associated errors and problems described, the method can use a registration-free method of training. Training can proceed by minimising or approximately minimising an objective function.

Given a score of how well the machine learning method predicts the sequence for each read of a target polymer, which is preferably the canonical sequence of the target polymer, an appropriate objective function can be created by combining the said scores and such a combination can be affected by applying some functional. Functionals that measure central trend are preferred. Examples of such functionals include: the mean score, the sum of all scores, the median score, trimmed-mean score, weighted-mean score, weighted sum of score quantiles (L-estimators), M-estimators for location.

Where the registration between the read and the canonical sequence is known, an augmented sequence of labels that is the same length as the read can be created which consists of a label when a new label is to be emitted or a ‘blank’ state otherwise. We refer to this augmented sequence of labels as a ‘labelling’ for the read. The score for this labelling can be calculated using one of many standard techniques in the art.

By way of example, a ‘read’ can be scored by combining the scores, for all possible labellings that are consistent with the canonical sequence, into a single score. Training in the case where the registration is known, or assumed known, is equivalent to the objective function being the individual score for that specific labelling.

The contribution of each individual score to the combined score may be weighted and, where the weight is zero, the calculation of the individual score need not be performed and so the overall calculation requires less computation resource than would be the case for the full calculation. An example of how weights can be usefully assigned is to only use a non-zero weight for those label assignments where the registration between the signal and canonical sequence stays entirely within a defined region.

Alternatively, weights could be used to favour assignments of labels whose metrics are consistent with an expectation of how the system should behave, for example, the global rate of translocation of the strand through the pore or local properties of the motor mechanics.

For several methods of combination, the score for a read can be calculated in an efficient manner, without explicit calculation of the individual scores for each possible labelling, using a dynamic programming technique. An example of one such application of this dynamic programming is in the training of the neural network in the Connectionist Temporal Classification (CTC) method for unsegmented sequence labelling [https://www.cs.toronto.edu/˜graves/icml_2006.pdf] and this approach has been directly applied to nanopore sequencing by the Chiron base calling software [https://academic.oup.com/gigscience/article/7/5/giy037/4966989].

An example of an efficient way of summing over all labellings can include a machine learning technique that predicts a weight Wᵣ(s,t) at every position of the read r that there is a transition from state s to state t between that position and the next or Wᵣ(s,-) for emitting a blank while in state s. The weights are normalised such that the combination over all possible labellings, regardless of canonical sequence, is a constant value.

To combine the scores for all labellings that agree with the canonical sequence, the method can perform dynamic programming through a grid with the read on one axis and the canonical sequence on the other. Each possible labelling which is equivalent to a monotonic path through this grid (strictly monotonic through the read axis, non-decreasing along the sequence axis).

FIG. 19 shows how three such paths arise in a simple case. The score for all labellings is accumulated using a frontier that progresses in strict succession through the positions of the read. The accumulation from one position in the read has two components: moving to the next position in the canonical sequence, with the associated weight, or staying in the same position with the weight associated with a ‘blank’. Letting cₛ be the label associated with position s of the canonical sequence, the combined score can be calculated recursively, as follows, using two operators (oplus) and (otimes)

$${f}_{k + 1} ( s + 1 ) = ⊕ ( \begin{matrix}{f}_{k} ( s ) & ⊗ & {W}_{k} ( {c}_{s} , {c}_{s + 1} ) \\ {f}_{k} ( s + 1 ) & ⊗ & {W}_{k} ( {c}_{s + 1} - )\end{matrix} ) \begin{matrix}Move \\ Stay\end{matrix}$$

The progress of the calculation is shown pictorially in FIG. 20.

In this framework, the score S(1) for a specific labelling l1, . . . , ln can be calculated by the combining the appropriate weights together as:

S(l)=W₁(l₀,l₁)⊗W₂(l₁,l₂)⊗ . . . ⊗Wₙ(lₙ₋₁,lₙ)

Using the operators oplus and otimes are log sum exp and ordinary summation respectively, where log sump exp is defined as:

log sum exp(x₁, . . . ,xₙ)=log Σᵢ₌₁ⁿe^{x}^{ᵢ}

Equivalently log sum exp(x₁, . . . ,xₙ)=x_{M}+log Σᵢ₌₁ⁿe^{x}^{ᵢ}^{-x}^{_{M}}

where x_{M}=maxᵢ xᵢ

Alternatively, the operations for combination may be maximum and summation; alternatively, the operators may be summation and multiplication; alternatively, the log sum exp operation may incorporate a sharpening factor:

Standard log sum exp(x₁, . . . ,xₙ)=log Σᵢ₌₁ⁿe^{x}^{ᵢ}

Sharpened: log sum expₐ(x₁, . . . ,xₙ)=1/a log Σᵢ₌₁ⁿe^{ax}^{ᵢ}

It is preferable to perform the numerically more stable but otherwise equivalent calculation:

Standard log sum exp(x₁, . . . ,xₙ)=x_{M}+log Σᵢ₌₁ⁿe^{x}^{ᵢ}^{-x}^{_{M}}

Sharpened: log sum expₐ(x₁, . . . ,xₙ)=x_{M}+1/a log Σᵢ₌₁ⁿe^{a(x}^{ᵢ}^{-x}^{_{M}}⁾

where x_{M}=maxᵢ xᵢ

Where efficient methods of calculation are not available, the objective function may be approximated by numerical techniques or by simulation using Monte Carlo techniques or low discrepancy sequences.

To train the machine learning technique, a canonical sequence needs to be associated with each read from a representative set. Several methods to identify the underlying canonical sequence of bases may be employed in the training process. In most cases, the identification of canonical sequence may be strengthened by using additional information, such as comparison with a reference genome.

For example, the network may initially be trained using reads of strands prepared from a small number of unique DNA fragments for which the canonical sequence is known, and the origin of each read can be inferred from basic metrics e.g. total read length.

Alternatively, strands can be associated with a canonical sequence using a 1D² sequencing approach where the complementary strand contains only canonical bases, is base called by established methods, and then used to infer the canonical sequence of the strand containing alternative bases.

Alternatively, given a rudimentary base caller, that functions well enough such that the sequence of strands can be identified e.g. by alignment to a reference genome, these methods may be “boot strapped” to train a more accurate base caller on a more diverse training set.

Alternatively, strands comprising a lower proportion of alternative bases (e.g. lower percentages of each base, and/or fewer bases substituted), may be used such that they can be identified with a base caller that is not aware of the modifications. The resulting trained base caller can then be used to identify the canonical sequence of reads from strands containing a higher proportion of alternative bases, from which a further base caller can be trained. This process can be repeated with increasing proportion of alternative bases until the desired composition is reached.

Where a good ground truth is known for the location of the alterative bases, they can be treated as a canonical base for the purposes of the methods disclosed. Where substitution of alternative bases varies on a strand-to-strand basis, a bespoke canonical sequence could be used for each read in the training set.

As an alternative to training the machine learning approach to estimate the canonical sequence, it could be trained to estimate an encoding of the canonical sequence. Alternatively, the base calling method could be trained to estimate a related sequence, the amino acid sequence of the protein product that would be obtained from an mRNA strand for example.

The method can include determining a sequence of an original polymer or native polymer, and wherein native modifications are not called. This aspect of the method can be useful in circumstances where base modifications are present in the strand to be sequenced, but the desired result is the canonical base sequence.

An example of where the method is advantageous is in the sequencing of long strands for the assembly of large genomes and resolution on complex repeat regions. Natural DNA contains base modifications, 5-methyl-cytosine or 6-methyl-adenine for example, that are not canonical bases and the presence and location of these modifications can differ from individual to individual and, indeed, cell to cell within the same individual. At present, it is not possible to duplicate long fragments of DNA using techniques like PCR, which synthesise a complementary strand containing only canonical bases, so the sequencing of long fragments requires natural DNA as input. Natural DNA contains many alternative bases, including the possibility of bases whose presence are as yet unknown to science, so the techniques presented are desirable to improve the estimate the canonical sequence produced.

A further example would be the sequencing of RNA for the purposes of expression studies. While creating duplicate strands containing only canonical bases is possible, methods used to achieve this have biases which change the composition of sample and so affect the quality of study. Base calling the natural strands directly is desirable to avoid bias.

Depending on the composition of the training set used, the trained base calling method implicitly incorporates knowledge about the types of alternative bases that may be present in natural samples and the context in which they are likely to occur, and this implicit knowledge is used to improve the estimate of the canonical sequence made. The effect of the implicit knowledge can be strengthened through the nature of the training set: for example, specific base callers can be trained for groups of organisms that are known to be predictable modification pattern (e.g. methylation of CpG in vertebrates).

Examination of intermediate calculations with the trained base caller, the pattern of activations in a neural network for example, can reveal where the network is using its implicit knowledge about alternative bases and so be used to infer their presence and location.

As described above the accuracy of nanopore sequencing can be improved by analysing polymers, or strands, comprising canonical and non-canonical polymer units. Improving base calling using machine learning, as described below, can be improved upon further by analysing polymers having canonical and non-canonical polymer units, as described and claimed.

In the case of a polypeptide, the polymer units may be amino acids that are naturally occurring or synthetic.

In the case of a polysaccharide, the polymer units may be monosaccharides.

Particularly where the measurement system 2 comprises a nanopore and the polymer comprises a polynucleotide, the polynucleotide may be long, for example at least 5 kB (kilo-bases), i.e. at least 5,000 nucleotides, or at least 30 kB (kilo-bases), i.e. at least 30,000 nucleotides, or at least 100 kB (kilo-bases), i.e. at least 100,000 nucleotides.

The nature of the measurement system 2 and the resultant measurements is as follows.

The measurement system 2 is a nanopore system that comprises one or more nanopores. In a simple type, the measurement system 2 has only a single nanopore, but a more practical measurement systems 2 employ many nanopores, typically in an array, to provide parallelised collection of information.

The measurements may be taken during translocation of the polymer with respect to the nanopore, typically through the nanopore. Thus, successive measurements are derived from successive portions of the polymer.

The nanopore is a pore, typically having a size of the order of nanometres, that may allows the passage of polymers therethrough.

A property that depends on the polymer units translocating with respect to the pore may be measured. The property may be associated with an interaction between the polymer and the pore. Such an interaction may occur at a constricted region of the pore.

The nanopore may be a biological pore or a solid state pore. The dimensions of the pore may be such that only one polymer may translocate the pore at a time.

The pore may be a DNA origami pore such as described in WO 2013/083983.

Where the nanopore is a biological pore, it may have the following properties.

The biological pore may be a transmembrane protein pore. Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from Msp or from α-hemolysin (α-HL). The transmembrane pore may be derived from lysenin. Suitable pores derived from lysenin are disclosed in WO 2013/153359. Suitable pores derived from MspA are disclosed in WO-2012/107778. The pore may be derived from CsgG, such as disclosed in WO-2016/034591.

The biological pore may be a naturally occurring pore or may be a mutant pore. Typical pores are described in WO-2010/109197, Stoddart D et al., Proc Natl Acad Sci, 12; 106(19):7702-7, Stoddart D et al., Angew Chem Int Ed Engl. 2010; 49(3):556-9, Stoddart D et al., Nano Lett. 2010 Sep. 8; 10(9):3633-7, Butler T Z et al., Proc Natl Acad Sci 2008; 105(52):20647-52, and WO-2012/107778.

The biological pore may be one of the types of biological pores described in WO-2015/140535 and may have the sequences that are disclosed therein.

The biological pore may be inserted into an amphiphilic layer such as a biological membrane, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in Gonzalez-Perez et al., Langmuir, 2009, 25, 10447-10450 or WO2014/064444. Alternatively, a biological pore may be inserted into a solid state layer, for example as disclosed in WO2012/005857.

A suitable apparatus for providing an array of nanopores is disclosed in WO-2014/064443. The nanopores may be provided across respective wells wherein electrodes are provided in each respective well in electrical connection with an ASIC for measuring current flow through each nanopore. A suitable current measuring apparatus may comprise the current sensing circuit as disclosed in PCT Patent Application No. PCT/GB2016/051319

The nanopore may comprise an aperture formed in a solid state layer, which may be referred to as a solid state pore. The aperture may be a well, gap, channel, trench or slit provided in the solid state layer along or into which analyte may pass. Such a solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al203, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO-2009/035647, WO-2011/046706 or WO-2012/138357. Suitable methods to prepare an array of solid state pores is disclosed in WO-2016/187519.

Such a solid state pore is typically an aperture in a solid state layer. The aperture may be modified, chemically, or otherwise, to enhance its properties as a nanopore. A solid state pore may be used in combination with additional components which provide an alternative or additional measurement of the polymer such as tunneling electrodes (Ivanov A P et al., Nano Lett. 2011 Jan. 12; 11(1):279-85), or a field effect transistor (FET) device (as disclosed for example in WO-2005/124888). Solid state pores may be formed by known processes including for example those described in WO-00/79257.

In one type of measurement system 2, there may be used measurements of the ion current flowing through a nanopore. These and other electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and WO-2000/28312. Alternatively, electrical measurements may be made using a multi-channel system, for example as described in WO-2009/077734, WO-2011/067559 or WO-2014/064443.

Ionic solutions may be provided on either side of the membrane or solid state layer, which ionic solutions may be present in respective compartments. A sample containing the polymer analyte of interest may be added to one side of the membrane and allowed to move with respect to the nanopore, for example under a potential difference or chemical gradient. Measurements may be taken during the movement of the polymer with respect to the pore, for example taken during translocation of the polymer through the nanopore. The polymer may partially translocate the nanopore.

In order to allow measurements to be taken as the polymer translocates through a nanopore, the rate of translocation can be controlled by a polymer binding moiety. Typically the moiety can move the polymer through the nanopore with or against an applied field. The moiety can be a molecular motor using for example, in the case where the moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, moieties that interact with that polymer type can be used. The polymer interacting moiety may be any disclosed in WO-2010/086603, WO-2012/107778, and Lieberman K R et al, J Am Chem Soc. 2010; 132(50):17961-72), and for voltage gated schemes (Luan B et al., Phys Rev Lett. 2010; 104(23):238103).

The polymer binding moiety can be used in a number of ways to control the polymer motion. The moiety can move the polymer through the nanopore with or against the applied field. The moiety can be used as a molecular motor using for example, in the case where the moiety is an enzyme, enzymatic activity, or as a molecular brake. The translocation of the polymer may be controlled by a molecular ratchet that controls the movement of the polymer through the pore. The molecular ratchet may be a polymer binding protein. For polynucleotides, the polynucleotide binding protein is preferably a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the target polynucleotide and controlling its movement through the pore. For instance, the enzyme may be modified to remove its enzymatic activity or may be used under conditions which prevent it from acting as an enzyme. Such conditions are discussed in more detail below.

Preferred polynucleotide handling enzymes are polymerases, exonucleases, helicases and topoisomerases, such as gyrases. The polynucleotide handling enzyme may be for example one of the types of polynucleotide handling enzyme described in WO-2015/140535 or WO-2010/086603.

Translocation of the polymer through the nanopore may occur, either cis to trans or trans to cis, either with or against an applied potential. The translocation may occur under an applied potential which may control the translocation.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as molecular brake slowing down the movement of a polynucleotide through the pore. Any moieties, techniques or enzymes described in WO-2012/107778 or WO-2012/033524 could be used to control polymer motion.

However, the measurement system 2 may be of alternative types that comprise one or more nanopores.

Similarly, the measurements may be of types other than measurements of ion current. Some examples of alternative types of measurement include without limitation: electrical measurements and optical measurements. A suitable optical method involving the measurement of fluorescence is disclosed by J. Am. Chem. Soc. 2009, 131 1652-1653. Possible electrical measurements include: current measurements, impedance measurements, tunneling measurements (for example as disclosed in Ivanov A P et al., Nano Lett. 2011 Jan. 12; 11(1):279-85), and FET measurements (for example as disclosed in WO2005/124888). Optical measurements may be combined with electrical measurements (Soni G V et al., Rev Sci Instrum. 2010 January; 81(1):014301). The measurement may be a transmembrane current measurement such as measurement of ion current flow through a nanopore. The ion current may typically be the DC ion current, although in principle an alternative is to use the AC current flow (i.e. the magnitude of the AC current flowing under application of an AC voltage).

Herein, the term ‘k-mer’ refers to a group of k-polymer units, where k is a positive plural integer. In many measurement systems, measurements may be dependent on a portion of the polymer that is longer than a single polymer unit, for example a k-mer although the length of the k-mer on which measurements are dependent may be unknown. In many cases, the measurements produced by k-mers or portions of the polymer having different identities are not resolvable.

In many types of the measurement system 2, the series of measurements may be characterised as comprising measurements from a series of events, where each event provides a group of measurements. The group of measurements from each event have a level that is similar, although subject to some variance. This may be thought of as a noisy step wave with each step corresponding to an event.

The events may have biochemical significance, for example arising from a given state or interaction of the measurement system 2. For example, in some instances, the event may correspond to interaction of a particular portion of the polymer or k-mer with the nanopore, in which case the group of measurements is dependent on the same portion of the polymer or k-mer. This may in some instances arise from translocation of the polymer through the nanopore occurring in a ratcheted manner.

Within the limits of the sampling rate of the measurements and the noise on the signal, the transitions between states can be considered instantaneous, thus the signal can be approximated by an idealised step trace. However when translocation rates approach the measurement sampling rate, for example, measurements are taken at 1 times, 2 times, 5 times or 10 times the translocation rate of a polymer unit, this approximation may not be as applicable as it was for slower sequencing speeds or faster sampling rates.

In addition, typically there is no a priori knowledge of number of measurements in the group, which varies unpredictably.

These two factors of variance and lack of knowledge of the number of measurements can make it hard to distinguish some of the groups, for example where the group is short and/or the levels of the measurements of two successive groups are close to one another.

The group of measurements corresponding to each event typically has a level that is consistent over the time scale of the event, but for most types of the measurement system 2 will be subject to variance over a short time scale.

Such variance can result from measurement noise, for example arising from the electrical circuits and signal processing, notably from the amplifier in the particular case of electrophysiology. Such measurement noise is inevitable due the small magnitude of the properties being measured.

Such variance can also result from inherent variation or spread in the underlying physical or biological system of the measurement system 2, for example a change in interaction, which might be caused by a conformational change of the polymer.

Most types of the measurement system 2 will experience such inherent variation to greater or lesser extents. For any given types of the measurement system 2, both sources of variation may contribute or one of these noise sources may be dominant.

With increase in the sequencing rate, being the rate at which polymer units translocate with respect to the nanopore, then the events may become less pronounced and hence harder to identify, or may disappear. Thus, analysis methods that rely on event detection may become less efficient at as the sequencing rate increases.

Increasing the measurement sampling rate may compensate for difficulties in measuring transitions but such faster sampling typically comes with a penalty in signal-to-noise.

The methods described below are effective even at relatively high sequencing rates, including sequencing rates at which the series of measurements are a series of measurements taken at a rate of at least 10 polymer units per second, preferably 100 polymer units per second, more preferably 500 polymer units per second, or more preferably 1000 polymer units per second.

The analysis system 3 will now be considered.

Herein, reference is made to posterior probability vectors and matrices that represent “posterior probabilities” of different sequences of polymer units or of different changes to sequences of polymer units. The values of the posterior probability vectors and matrices may be actual probabilities (i.e. values that sum to one) or may be weights or weighting factors which are not actual probabilities but nonetheless represent the posterior probabilities. Generally, where the values of the posterior probability vectors and matrices are expressed as weights or weighting factors, the probabilities could in principle be determined therefrom, taking account of the normalisation of the weights or weighting factors. Such a determination may consider plural time-steps. By way of non-limitative example, two methods are described below, referred to as local normalisation and global normalisation.

Similarly, reference is made to scores representing the probability of the series of polymer units that are measured being reference series of polymer units. In the same way, the value of the score may be an actual probability or may be a weight that is not an actual probability but nonetheless represents the probability of the series of polymer units that are measured being reference series of polymer units.

The analysis system 3 may be physically associated with the measurement system 2, and may also provide control signals to the measurement system 2. In that case, the nanopore measurement and analysis system 1 comprising the measurement system 2 and the analysis system 3 may be arranged as disclosed in any of WO-2008/102210, WO-2009/07734, WO-2010/122293, WO-2011/067559 or WO2014/04443.

Alternatively, the analysis system 3 may implemented in a separate apparatus, in which case the series of measurement is transferred from the measurement system 2 to the analysis system 3 by any suitable means, typically a data network. For example, one convenient cloud-based implementation is for the analysis system 3 to be a server to which the input signal 11 is supplied over the internet.

The analysis system 3 may be implemented by a computer apparatus executing a computer program or may be implemented by a dedicated hardware device, or any combination thereof. In either case, the data used by the method is stored in a memory in the analysis system 3.

In the case of a computer apparatus executing a computer program, the computer apparatus may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory.

In the case of the computer apparatus being implemented by a dedicated hardware device, then any suitable type of device may be used, for example an FPGA (field programmable gate array) or an ASIC (application specific integrated circuit).

A method of using the nanopore measurement and analysis system 1 is performed as follows.

Firstly, the series of measurements are taken using the measurement system 2. For example, the polymer is caused to translocate with respect to the pore, for example through the pore, and the series of measurements are taken during the translocation of the polymer. The polymer may be caused to translocate with respect to the pore by providing conditions that permit the translocation of the polymer, whereupon the translocation may occur spontaneously.

Secondly, the analysis system 3 performs a method of analysing the series of measurements as will now be described. There will first be described a basic method, and then some modifications to the basic method.

The basic method analyses the series of measurements using a machine learning technique, which in this example is a recurrent neural network. The parameters of the recurrent neural network take values during the training that is described further below, and as such the recurrent neural network is not dependent on the measurements having any particular form or the measurement system 2 having any particular property. For example, the recurrent neural network is not dependent on the measurements being dependent on k-mers.

The basic method uses event detection as follows.

The basic method processes the input as a sequence of events that have already been determined from the measurements (raw signal) from the measurement system 2. Thus, the method comprises initial steps of identifying groups of consecutive measurements in the series of measurements as belonging to a common event, and deriving a feature vector comprising one or more feature quantities from each identified group of measurements, as follows.

The segmentation of the raw samples into events uses the same method as described in WO 2015/140535, although it not thought that the basic method is sensitive to the exact method of segmentation.

However, for completeness, an outline of a segmentation process that may be applied is described as follows with reference to FIG. 2. FIG. 2 shows the a graph of the raw signal 20 which comprises the series of measurements, having with step-like ‘event’ behaviour, a sliding pair of windows 22, a sequence of pairwise t-statistics 23 calculated from the raw signal 20, showing localized peaks, and a threshold 24 (dashed line), and a set of event boundaries 25 corresponding to the peaks.

Groups of consecutive measurements are identified as belonging to a common event as follows. The consecutive pair of windows 21 are slid across the raw signal 20 and the pairwise t-statistic of whether the samples (measurements) in one window 21 have a different mean to the other is calculated at each position, giving the sequences of statistics 23. A thresholding technique against the threshold 24 is used to localise the peaks 23 in the sequence of statistics 23 that correspond to significant differences in level of the original raw signal 20, which are deemed to be event boundaries 25, and then the location of the peaks 23 is determined using a standard peak finding routine, thereby identifying the events in the series of measurements of the raw signal 20.

Each event is summarised by deriving, from each identified group of measurements, a set of one or more feature quantities that describe its basic properties. An example of three feature quantities that may be used are as follows and are shown diagrammatically in FIG. 3:
Level L: a measure of the average current for the event, generally the mean but could be a median or related statistics.
Variation V: how far samples move away from the central level, generally the standard deviation or variance of the event. Other alternatives include the Median Absolute Deviation or the mean deviation from the median.
Length (or dwell) D: How long the event is, either as a number of samples or in seconds.

In general, any one or more feature quantities may be derived and used. The one or more feature quantities comprise a feature vector.

As with any analysis of a noisy process, the segmentation may make mistakes. Event boundaries may be missed, resulting in events containing multiple levels, or additional boundaries may be created where none should exist. Over-segmentation, choosing an increase in false boundaries over missing real boundaries, has been found to result in better basecalls.

The feature vector comprising one or more feature quantities are operated on by the recurrent neural network as follows.

The basic input to the basic method is a time-ordered set of feature vectors corresponding to events found during segmentation. As is standard practice with most machine learning procedures, the input features are normalised to help stabilise and accelerate the training process but the basic method has two noticeable differences: firstly, because of the presence of significant outlier events, Studentisation (centre by mean and scale by standard deviation) is used rather than the more common min-max scaling; a second, more major change, is that that scaling happens on a per-read basis rather than the scaling parameters being calculated over all the training data and then fixed.

Other alternatives to min-max scaling, designed to be robust to extreme values, may also be applied. Examples of such a method would be a min-max scaling whose parameters are determined after trimming the lowest and highest x % of values, or scaling based on the median and median absolute deviation.

The reason for this deviation from the standard training protocol is to help the network generalise to the variation across devices that will be encountered in the field. While the number of reads that can be trained from is extremely large, time and cost considerations mean that they will have come from a small number of devices and so the training run conditions represent a small section of those that might be encountered externally. Per-read normalisation helps the network generalise, although there is a potential loss in accuracy.

A fourth ‘delta’ feature, derived from the others, is also used as input to the basic method, intended to represent how different neighbouring events are from each other and so indicate whether there is a genuine change of level or whether the segmentation was incorrect.

The exact description of the delta feature has varied between different implementations of the basic method, and a few are listed below, but the intention of the feature remains the same.
Absolute difference in levels, followed by normalisation.
Squared difference in levels, followed by normalisation.
Difference in levels, followed by partial normalisation (scaled but not centred).

The basic method uses a deep neural network consisting of multiple bidirectional recurrent layers with sub-sampling. An overview of the architecture of a recurrent neural network 30 that may be implemented in the analysis system 3 is shown in FIG. 4 and arranged as follows, highlighting many of the features that differentiate from an analysis performed using an HMM.

In overview, the recurrent neural network 30 comprises: a windowing layer 32 that performs windowing over the input events; a bidirectional recurrent layers 34 that process their input iteratively in both forwards and backwards directions; feed-forward layers 35 that may be configured as a subsampling layer to reduce dimensionality of the recurrent neural network 30; and a softmax layer 36 that performs normalization using a softmax process to produce output interpretable as a probability distribution over symbols. The analysis system 3 further includes a decoder 37 to which the output of the recurrent neural network 30 is fed and which performs a subsequent decoding step.

In particular, the recurrent neural network 30 receives the input feature vectors 31 and passes them through the windowing layer 32 which windows the input feature vectors 31 to derive windowed feature vectors 33. The windowed feature vectors 33 are supplied to the stack of plural bidirectional recurrent layers 34. Thus, the influence of each input event is propagated throughout all steps of the model represented in the recurrent neural network 30 at least twice with the second pass informed by the first. This double bidirectional architecture allows the recurrent neural network 30 to accumulate and propagate information in a manner unavailable to HMMs. One consequence of this is that the recurrent neural network 30 doesn't require an iterative procedure to scale the model to the read.

Two bidirectional recurrent layers 34 are illustrated in this example, differentiated as 34-1 and 34-2 and each followed by a feed-forward layer 35, differentiated as 35-1 and 35-2, but in general there may be any plural number of bidirectional recurrent layers 34 and subsequent feed-forward layers 35.

The output of the final feed-forward layer 35-2 is supplied to the softmax layer 36 which produces outputs representing posterior probabilities that are supplied to the decoder 37. The nature of these posterior probabilities and processing by the decoder 37 are described in more detail below.

By way of comparison, a HMM 50 can be described in a form similar to a neural network, as shown in FIG. 5. The HMM 50 includes inputs single events, with no windowing and no delta feature and comprises: a forwards-backwards layer 54 into which the feature vectors 51 are fed and which performs forwards and backwards passes of the network with tightly coupled parameters; an additive combination layer 55 into which the output of the forwards-backwards layer 54 is fed and which performs subsampling by element-wise addition of the output of the forward and backward passes; a normalisation layer 56 that performs normalization to produce out-put interpretable as a probability distribution over symbols; and a decoder 57 that performs a subsequent decoding step.

Due to their assumption that the emission of the HMM 50 is completely described by the hidden state, the HMM 50 cannot accept windowed input and nor can they accept delta-like features since the input for any one event is assumed to be statistical independent from another given knowledge of the hidden state (although optionally this assumption may be relaxed by use of an extension such as an autoregressive HMM). Rather than just applying the Viterbi algorithm directly to decode the most-likely sequence of states, the HMM for the nanopore sequence estimation problem proceeds via the classical forwards/backwards algorithm in the forwards-backwards layer 52 to calculate the posterior probability of the each hidden label for each event and then an addition Viterbi-like decoding step in the decoder 57 determines the hidden states. This methodology has been referred to as posterior-Viterbi in the literature and tends to result in estimated sequences where a greater proportion of the states are correctly assigned, compared to Viterbi, but still form a consistent path.

Table 1 summarizes the key differences between how the comparable layers are used in this and in the basic method, to provide a comparison of similar layers types in the architecture of the HMM 50 and the basic method, thereby highlighting the increased flexibility given by the neural network layers used in the basic method.

TABLE 1

| | | Basic method implemented in |
|---|---|---|
| Element | HMM 30 | recurrent neural network 30 |
| Windowing | Only a single event | A window of features |
| layer 32 | is permitted | can be used as |
| | as input and | input including |
| | features are | features that are |
| | assumed to be | dependent on other |
| | statistically | events, or even |
| | dependent from | globally on all events. |
| | event to event. | |
| Bidirectional | Parameters for | Flexible choice of |
| recurrent | the forwards- | unit architecture. |
| layers | backwards layer | The forwards and |
| 34 | 52 are tightly | backwards units may |
| | coupled and the | have different |
| | type of unit is | parameters, but |
| | defined by the | difference sizes or |
| | statistical model. | even be composed |
| | The size of the | of different unit types. |
| | forwards and | |
| | backwards must | |
| | be the same as | |
| | the number of | |
| | symbols to be | |
| | output. | |
| Subsampling | The output | Separate affine |
| in | vectors for the | transforms are applied |
| feed-forward | forward-backwards | to the output vectors |
| layers 35 | layer 52 at | for the forward |
| | each column are | and backwards |
| | summed in the | layer at each column, |
| | normalisation | followed by |
| | layer 56 to create | summation; this is |
| | a single vector | equivalent to |
| | which has the | applying an affine |
| | same size as the | transform to the |
| | units in the | vector formed by |
| | forwards-backwards | concatenation of the |
| | layer 52 | input and output. |
| | (i.e. the number | An activation |
| | of symbols). | function is then applied |
| | | element-wise to the |
| | | resultant matrix. |
| Normalisation | Normalisation in the | The ‘softmax’ |
| in softmax | normalization | functor is applied to |
| layer | layer 56 is performed | each column after |
| 36 | by applying the | applying a general |
| | ‘softmax’ functor, that is | affine transform to |
| | the same as the ‘softmax’ | project the input |
| | unit 36 described herein | vector into a space |
| | but without an | with dimension |
| | affine transform, | equal to the number |
| | to each column | of possible output |
| | of the output. | symbols. |

While there are the same number of columns output as there are events, it is not correct to assume that each column is identified with a single event in the input to the network since its contents are potentially informed by the entire input set of events because of the presence of the bidirectional layers. Any correspondence between input events and output columns is through how they are labelled with symbols in the training set.

The bidirectional recurrent layers 34 of the recurrent neural network 30 may use several types of neural network unit as will now be described. The types of unit fall into two general categories depending on whether or not they are ‘recurrent’. Whereas non-recurrent units treat each step in the sequence independently, a recurrent unit is designed to be used in a sequence and pass a state vector from one step to the next. In order to show diagrammatically the difference between non-recurrent units and recurrent units, FIG. 6 shows a non-recurrent layer 60 of non-recurrent units 61 and FIGS. 7 to 9 show three different layers 62 to 64 of respective non-recurrent units 64 to 66. In each of FIGS. 6 to 9, the arrows show connections along which vectors are passed, arrows that are split being duplicated vectors and arrows which are combined being concatenated vectors.

In the non-recurrent layer 60 of FIG. 6, the non-recurrent units 61 have separate inputs and outputs which do not split or concatenate.

The recurrent layer 62 of FIG. 7 is a unidirectional recurrent layer in which the output vectors of the recurrent units 65 are split and passed to unidirectionally to the next recurrent unit 65 in the recurrent layer.

While not a discrete unit in its own right, the bidirectional recurrent layers 63 and 64 of FIGS. 8 and 9 each have a repeating unit-like structure made from simpler recurrent units 66 and 67, respectively.

In the bidirectional recurrent layer of FIG. 8, the bidirectional recurrent layer 63 consists of two sub-layers 68 and 69 of recurrent units 66, being a forwards sub-layer 68 having the same structure as the unidirectional recurrent layer 62 of FIG. 7 and a backward sub-layer 69 having a structure that is reversed from the unidirectional recurrent layer 62 of FIG. 7 as though time were reversed, passing state vectors from one unit 66 to the previous unit 66. Both the forwards and backwards sub-layers 68 and 69 receive the same input and their outputs from corresponding units 66 are concatenated together to form the output of the bidirectional recurrent layer 63. It is noted that there are no connections between any unit 66 within the forwards sub-layer 68 and any unit within the backwards sub-layer 69.

The alternative bidirectional recurrent layer 64 of FIG. 9 similarly consists of two sub-layers 70 and 71 of recurrent units 67, being a forwards sub-layer 68 having the same structure as the unidirectional recurrent layer 62 of FIG. 7 and a backwards sub-layer 69 having a structure that is reversed from the unidirectional recurrent layer 62 of FIG. 7 as though time were reversed. Again the forwards and backwards sub-layers 68 and 69 receive the same inputs, However, in contrast to the bidirectional recurrent layer of FIG. 8, the outputs of forwards sub-layer 68 are the inputs of the backwards sub-layer 69 and the outputs of the backwards sub-layer 69 form the output of the bidirectional recurrent layer 64 (the forwards and backwards sub-layers 68 and 69 could be reversed).

A generalisation of the bidirectional recurrent layer shown in FIG. 9 would be a stack of recurrent layers consisting of plural ‘forwards’ and ‘backward’ recurrent sub-layers, where the output of each layer is the input for the next layer.

The bidirectional recurrent layers 34 of FIG. 3 may take the form of either of the bidirectional recurrent layers 63 and 64 of FIGS. 8 and 9. In general, the bidirectional recurrent layers 34 of FIG. 3 could be replaced by a non-recurrent layer, for example the non-recurrent layer 60 of FIG. 6, or by a unidirectional recurrent layer, for example the recurrent layer 62 of FIG. 7, but improved performance is achieved by use of bidirectional recurrent layers 34.

The feed-forward layers 35 will now be described.

The feed-forward layers 35 comprise feed-forward units 38 that process respective vectors. The feed-forward units 38 are the standard unit in classical neural networks, that is an affine transform is applied to the input vector and then a non-linear function is applied element-wise. The feed-forward layers 35 all use the hyperbolic tangent for the non-linear function, although many others may be used with little variation in the overall accuracy of the network.

If the input vector at step t is Iₜ, and the weight matrix and bias for the affine transform are A and b respectively, then the output vector Oₜ is:

yₜ=AIₜ+b Affine transform

Oₜ=tanh(yₜ) Non-linearity

The output of the final feed-forward layer 35 is fed to the softmax layer 36 that comprises softmax units 39 that process respective vectors.

The purpose of the softmax units 39 is to turn an input vector into something that is interpretable as a probability distribution over output symbols, there being a 1:1 association with elements of the output vector and symbols. An affine transformation is applied to the input vector, which is then exponentiated element-wise and normalised so that the sum of all its elements is one. The exponentiation guarantees that all entries are positive and so the normalisation creates a valid probability distribution.

If the input vector at step t is 1, and the weight matrix and bias for the affine transform are A and b respectively, then the output vector Oₜ is:

yₜ=AIₜ+b Affine transform

zₜ=e^{y}^{ₜ} Exponentiation

Oₜ=zₜ/1′zₜ Normalisation

where 1′ is the transpose of the vector whose elements are all equal to the unit value, so 1′x is simply the (scalar) sum of all the elements of x.

Use of the softmax layer 36 locally normalises the network's output at each time-step. Alternatively, the recurrent neural net 30 may be normalised globally across over all time steps so that the sum over all possible output sequences is one. Global normalisation is strictly more expressive than local normalisation and avoids an issue known in the art as the ‘label bias problem’.

The advantages of using global normalisation over local normalisation are analogous to those that Conditional Random Fields (Lafferty et al., Conditional Random Fields: Probabilistic Models for Segmenting and Labeling Sequence Data, Proceedings of the International Conference on Machine Learning, June 2001) have over Maximum Entropy Markov Models (McCallum et al., Maximum Entropy Markov Models for Information Extraction and Segmentation, Proceedings of ICML 2000, 591-598. Stanford, Calif., 2000). The label bias problem affects models in which the matrix of allowed transitions between states is sparse, such as extensions to polymer sequences.

With local normalisation, the transition probabilities for each source state will be normalised to one, which causes states that have the fewest feasible transitions to receive high scores, even if they are a poor fit to the data. This creates a bias towards selecting states with a small number of feasible transitions.

Global normalisation alleviates this problem by normalising over the entire sequence, allowing transitions at different times to be traded against each other. Global normalisation is particularly advantageous for avoiding biased estimates of homopolymers and other low complexity sequences, as these sequences may have different numbers of allowed transitions compared to other sequences (it may be more or fewer, depending on the model).

The non-recurrent units 62 and recurrent units 65 to 67 treat each event independently, but may be replaced by Long Short-Term Memory units having a form as will now be described.

Long Short-Term Memory (LSTM) units were introduced in Hochreiter and Schmidhuber, Long short-term memory, Neural Computation, 9 (8): 1735-1780, 1997. An LSTM unit is a recurrent unit and so passes a state vector from one step in the sequence to the next. The LSTM is based around the notation that the unit is a memory cell: a hidden state containing the contents of the memory is passed from one step to the next and operated on via a series of gates that control how the memory is updated. One gate controls whether each element of the memory is wiped (forgotten), another controls whether it is replaced by a new value, and a final gate that determines whether the memory is read from and output. What makes the memory cell differentiable is that the binary on/off logic gates of the conceptual computer memory cell are replaced by notional probabilities produced by a sigmoidal function and the contents of the memory cells represent an expected value.

Firstly the standard implementation of the LSTM is described and then the ‘peep-hole’ modification that is actually used in the basic method.

The standard LSTM is as follows.

The probabilities associated with the different operations on the LSTM units are defined by the following set of equations. Letting Iₜ be input vector for step t, Oₜ be the output vector and let the affine transform indexed by x that has bias bₓ and weight matrices W_{xI} and W_{xO} for the input and previous output respectively; σ is the non-linear sigmoidal transformation.

fₜ=σ(W_{fI}Iₜ+W_{fO}Oₜ₋₁+b_{f}) Forget probability

uₜ=σ(W_{uI}Iₜ+W_{uO}Oₜ₋₁+bᵤ) Update probability

oₜ=σ(W_{oI}±W_{oO}Oₜ₋₁+bₒ) Output probability

Given the update vectors defined above and letting the ∘ operator represent element-wise (Hadamard) multiplication, the equations to update the internal state Sₜ and determine the new output are:

vₜ=tanh(W_{vI}Iₜ+W_{vO}Oₜ₋₁+bᵥ) Value to update with

Sₜ=Sₜ₋₁∘fₜ+vₜ∘uₜ Update memory cell

Oₜ=tanh(sₜ)∘oₜ Read from memory cell

The peep-hole modification is as follows.

The ‘peep-hole’ modification (Gers and Schmidhuber, 2000) adds some additional connections to the LSTM architecture allowing the forget, update and output probabilities to ‘peep at’ (be informed by) the hidden state of the memory cell. The update equations for the network are as above but, letting Pₓ be a ‘peep’ vector of length equal to the hidden state, the three equations for the probability vectors become:

fₜ=σ(W_{fI}Iₜ+W_{fO}Oₜ₋₁+b_{f}+P_{f}∘Sₜ₋₁) Forget probability

uₜ=σ(W_{uI}Iₜ+W_{uO}Oₜ₋₁+bᵤ+Pᵤ∘Sₜ₋₁) Update probability

oₜ=σ(W_{oI}Iₜ+W_{oO}Oₜ₋₁+bₒ+Pₒ∘Sₜ) Output probability

The non-recurrent units 62 and recurrent units 65 to 67 may alternatively be replaced by Gated Recurrent Units having a form as follows.

The Gated Recurrent Unit (GRU) has been found to be quicker to run but initially found to yield poorer accuracy. The architecture of the GRU is not as intuitive as the LSTM, dispensing with the separation between the hidden state and the output and also combining the ‘forget’ and ‘input gates’.

oₜ=σ(W_{oI}Iₜ+W_{oS}Sₜ₋₁+bₒ) Output probability

uₜ=Sₜ₋₁∘σ(W_{uI}Iₜ+W_{uS}Sₜ₋₁+bᵤ) Update from state

vₜ=tanh(W_{vI}Iₜ+W_{vR}uₜ+bᵥ) Value to update with

Sₜ=(1−oₜ)∘Sₜ₋₁+oₜ∘vₜ Update state

A HMM can be described as a neural unit as follows.

Although not used in the basic method, for completeness here is described how the forwards (backwards) HMM algorithm can be described using the recurrent neural network framework. A form whose output is in log-space is presented. A HMM is described by its transition matrix T and log density function δ parameterized by μ. The log-density function takes the input features and returns a vector of the log-probabilities of those features conditioned on the hidden state, the exact form of the function being specified by the parameters μ.

oₜ=δ(Iₜ;μ) Log density function

eₜ=exp(Sₜ₋₁) Exponentiate

fₜ=Tⁱeₜ Transition

Sₜ=oₜ+log fₜ Update state

As explained above, the recurrent neural network 30 produces outputs representing posterior probabilities that are supplied to a decoder 37. In the basic method the outputs are plural posterior probability vectors, each representing posterior probabilities of plural different sequences of polymer units. Each plural posterior probability vector corresponds to respective identified groups of measurements (events).

The decoder 37 derives an estimate of the series of polymer units from the posterior probability vectors, as follows.

The plural posterior probability vectors may be considered as a matrix with a column for each step, each column being a probability distribution over a set of symbols representing k-mers of predetermined length and an optional extra symbol to represent bad data (see ‘Bad events as handled as follows’ below). Since k-mers for neighbouring steps will overlap, a simple decoding process such as ‘argmax’, picking the k-mer that has the maximal probability at each step, and concatenating the result will result in a poor estimate of the underlying template DNA sequence. Good methods, the Viterbi algorithm for example, exist for finding the sequence of states that maximises the total score subject to restrictions on types of state-to-state transition that may occur.

If plural posterior probability vectors is the matrix, where the probability assigned to state j at step t is pₜⱼ, and there is set of transition weights τ_{i→j} for moving from state i to state j, then the Viterbi algorithm finds the sequence of states that maximises the score

$$L ( {s}_{1} … {s}_{T} ) = log {p}_{1 {ɛ}_{1}} + {\sum }_{t = 2}^{T} {τ}_{{s}_{t 1} \to {ɛ}_{1}} + log {p}_{2 {s}_{t}}$$

The Viterbi algorithm first proceeds in an iterative fashion from the start to end of the network output. The element fᵢⱼ of the forwards matrix represents the score of the best sequence of states up to step i ending in state j; element bij of the backwards matrix stores the previous state given that step i is in state j

$${f}_{0 s} = 0$$

$${f}_{is} = log {p}_{is} + {max}_{j} {τ}_{j \to s} + {f}_{i - 1 j}$$

$${b}_{is} = {argmax}_{j} {τ}_{j \to s} + {f}_{i - 1 j}$$

The best overall score can be determined by finding the maximal element of the final column T of the forward matrix; finding the sequence of states that achieves this score proceeds iteratively from the end to the start of the network output.

S_{T}=argmaxₛf_{Ts}

sᵢ=bᵢₛ_{ᵢ₊₁}

The transition weights define the allowed state-to-state transitions, a weight of negative infinity completely disallowing a transition and negative values being interpretable as a penalty that suppress that transition. The previously described ‘argmax’ decoding is equivalent to setting all the transition weights to zero. Where there are many disallowed transitions, a substantial runtime improvement can be obtained by performing the calculation in a sparse manner so only the allowed transitions are considered.

Having applied the Viterbi algorithm, each column output (posterior probability vector) by the network is labelled by a state representing a k-mer and this set of states is consistent.

The estimate of the template DNA sequence is formed by maximal overlap of the sequence of k-mers that the symbols represent, the transition weights having ensured that the overlap is consistent. Maximal overlap is sufficient to determine the fragment of the estimated DNA sequence but there are cases, homopolymers or repeated dimers for example, where the overlap is ambiguous and prior information must be used to disambiguate the possibilities. For our present nanopore device, the event detection is parametrised to over-segment the input and so the most likely overlap in ambiguous cases is the most complete.

Bad events are handled as follows.

The basic method emits on an alphabet that contains an additional symbol trained to mark bad events that are considered uninformative for basecalling. Events are marked as bad, using a process such as determining whether the ‘bad’ symbol is the one with the highest probability assigned to it or by a threshold on the probability assigned, and the corresponding column is removed from the output. The bad symbol is removed from the remaining columns and then they are individually renormalised so as to form a probability distribution over the remaining symbols. Decoding then proceeds as described above.

The recurrent neural network is trained for a particular type of measurement system 2 using techniques that are conventional in themselves and using training data in the form of series of measurements for known polymers.

Some modifications to the basic method will now be described.

The first modification relates to omission of event calling. Having to explicitly segment the signal into events causes many problems with base calling: events are missed or over called due to incorrect segmentation, the type of event boundaries that can be detected depends on the filter that has been specified, the form of the summary statistics to represent each event are specified up-front and information about the uncertainty of the event call is not propagated into the network. As the speed of sequencing increases, the notion of an event with a single level becomes unsound, the signal blurring with many samples straddling more than one level due the use of an integrating amplifier, and so a different methodology may be used to find alternative informative features from the raw signal.

Hence, the first modification is to omit event calling and instead perform a convolution of consecutive measurements in successive windows of the series of measurements to derive a feature vector in respect of each window, irrespective of any events that may be evident in the series of measurements. The recurrent neural network then operates on the feature vectors using said machine learning technique.

Thus, windows of measurements of fixed length, possibly overlapping, are processed into feature vectors comprising plural feature quantities that are then combined by a recurrent neural network and associated decoder to produce an estimate of the polymer sequence. As a consequence, the output posterior probability matrices corresponding to respective measurements or respective groups of a predetermined number of measurements depend on the degree of down-sampling in the network.

FIG. 10 shows an example of the first modification. In particular, FIG. 10 shows graph of the raw signal 20 which comprises the series of measurements, and an input stage 80 that may be arranged in front of the recurrent neural network 30 described above.

The input stage 80 feeds measurements in overlapping windows 81 into feature detector units 82. Thus, the raw signal 20 is processed in fixed length windows by the feature detector units 82 to produce the feature vector of features for each window, the features taking the same form as described above. The same feature detection unit is used for every window. The sequence of feature vectors produced is fed sequentially into the recurrent neural network 30 arranged as described above to produce a sequence estimate.

The feature detector units 82 are trained together with the recurrent neural network 30.

An example of a feature detector implemented in the feature detector units 82 is a single layer convolutional neural network, defined by an affine transform with weights W and bias h, and an activation function g. Here t_{t-j:t+k} represents a window of measurements of the raw signal 20 containing the t−j to the t+k measurements inclusive, and Oₜ is the output feature vector.

yₜ=AI_{t-j:t+k}+b Affine transform

Oₜ=g(yₜ) Activation

The hyperbolic tangent is a suitable activation function but many more alternatives are known in the art, including but not restricted to: the Rectifying Linear Unit (ReLU), Exponential Linear Unit (ELU), softplus unit, and sigmoidal unit. Multi-layer neural networks may also be used as feature detectors.

A straight convolutional network, as described, has the disadvantage that there is a dependence on the exact position of detected features in the raw signal and this also implies a dependence on the spacing between the features. The dependence can be alleviated by using the output sequence of feature vectors generated by the first convolution as input into a second ‘pooling’ network that acts on the order statistics of its input.

By way of example, where the pooling network is a single layer neural network, the following equations describe how the output relates to the input vectors. Letting f be an index over input features, so A_{f} is the weight matrix for feature f, and let [symbol] be a functor that returns some or all of the order statistics of its input:

$${y}_{t} = b + {\sum }_{f} {A}_{f} S ( {I}_{f , t - j , t + k} ) Affine transform$$

$${O}_{t} = g ( {y}_{t} ) Activation$$

One useful yet computationally efficient example of such a layer is that which returns a feature vector, the same size as the number of input features, whose elements are the maximum value obtained for each respective feature. Letting the functor [symbol] _{M} return only the last order statistic, being the maximum value obtained in its input, and letting U_{f} be the (single column) matrix that consists entirely of zeros other than a unit value at its (fₓ 1) element:

$${y}_{t} = {\sum }_{f} {U}_{f} {S}_{M} ( {I}_{f , t - j : t + k} ) Affine transform$$

$${O}_{t} = {y}_{t} No activation applied$$

Since the matrices U_{f} are extremely sparse, for reasons of computation efficiency, the matrix multiplications may be performed implicitly: here effect of Σ_{f} U_{f}x_{f} is to set element f of the output feature vector to x_{f}.

The convolutions and/or pooling may be performed only calculating their output for every nth position (a stride of n) and so down-sampling their output. Down-sampling can be advantageous from a computational perspective since the rest of the network has to process fewer blocks (faster compute) to achieve a similar accuracy.

Adding a stack of convolution layers solves many of the problems described above: the feature detection learned by the convolution can function both as nanopore-specific feature detectors and summary statistics without making any additional assumptions about the system; feature uncertainty is passed down into the rest of the network by relative weights of different features and so further processing can take this information into account leading to more precise predictions and quantification of uncertainty.

The second modification relates to the output of the recurrent neural network 30, and may optionally be combined with the first modification.

A possible problem for decoding the output of the basic method implemented in the recurrent neural network 30 is that, once the highest-scoring path through the k-mers has been determined, the estimate of the polymer sequence still has be determined by overlap and this process can be ambiguous.

To highlight the problem, consider the case where the history of the process is moving through a homopolymer region: all overlaps between the two k-mers are possible and several are feasible, corresponding to an additional sequence fragment of zero, one or two bases long for example. A strategy that relies on k-mers only partially solves the sequence estimation problem.

Thus, the second modification is to modify the outputs of the recurrent neural network 30 representing posterior probabilities that are supplied to the decoder 37. In particular, the ambiguity is resolved by dropping the assumption of decoding into k-mers and so not outputting posterior probability vectors that represent posterior probabilities of plural different sequences of polymer units. Instead, there is output posterior probability matrices, each representing, in respect of different respective historical sequences of polymer units corresponding to measurements prior or subsequent to the respective measurement, posterior probabilities of plural different changes to the respective historical sequence of polymer units giving rise to a new sequence of polymer units, as will now be described.

The historical sequences of polymer units are possible identities for the sequences that are historic to the sequence presently being estimated, and the new sequence of polymer units is the possible identity for the sequence that is presently being estimated for different possible changes to the historical sequence. Posterior probabilities for different changes from different historical sequences are derived, and so form a matrix with one dimension in a space representing all possible identities for the historical sequence and one dimension in a space representing all possible changes.

Notwithstanding the use of the term “historical”, historical sequences of polymer units corresponding to measurements prior or subsequent to the respective measurement, as the processing is effectively reversible and may proceed in either direction along the polymer.

Possible changes that may be considered are:
changes that remove a single polymer unit from the beginning or end of the historical sequence of polymer units and a single polymer unit to the end or beginning of the historical sequence of polymer units.
changes that remove two or more polymer units from the beginning of the historical sequence of polymer units and add two or more polymer units to the end of the historical sequence of polymer units.
a null change.

This will now be considered in more detail.

The second modification will be referred to herein as implementing a “transducer” at the output stage of the recurrent neural network 30. In general terms, the input to the transducer at each step is a posterior probability matrix that contains values representing posterior probabilities, which values may be weights, each associated with moving from a particular history-state using a particular movement-state. A second, predetermined matrix specifies the destination history-state given the source history-state and movement-state. The decoding of the transducer implemented in the decoder 37 may therefore find the assignment of (history-state, movement-state) to each step that maximises the weights subject to the history-states being a consistent path, consistent defined by the matrix of allowed movements.

By way of illustration, FIG. 11 shows how the output of the recurrent neural network that is input to the decoder 36 may be generated in the form of a posterior probability matrices 40 from the feature vectors 31 that are input to the recurrent neural network 30. FIG. 12 illustrates an example of the result of decoding into a tuple of history-state 41 and movement-state 42 when the space of the history-state is 3-mers and the space of the movement-state 42 is sequence fragments. In particular, FIG. 12 illustrates four successive history-states 41 and movement-states 42 and it can be seen how the history state 41 changes in accordance with the change represented by the movement-state 42.

The second modification provides a benefit over the basic method because there are some cases where the history-states 41 (which is considered alone in the basic method) are ambiguous as to the series of polymer units, whereas the movement states 42 are not ambiguous. By way of illustration, FIG. 13 shows some sample cases where just considering the overlap between states on the highest scoring path, analogously to the basic method, results in an ambiguous estimate of the series of polymer units, whereas the sequence fragments of the movement-states 42 used in the second medication are not ambiguous

The modification of the Viterbi algorithm that may be used for decoding is below but, for clarity, we first consider some concrete examples of how transducers may be used at the output of the softmax layer 56 and what their sets of history-states 41 and movement-states 42 might look like.

In one use of transducers, the set of history-states 41 is short sequence fragments of a fixed length and the movement-states are all sequence fragments up to a possible different fixed length, e.g. fragments of length three and up to two respectively means that the input to the decoding at each step is a weight matrix of size 4³×(1+4+4²). The history-states 41 are {AAA, AAC, . . . TTT} and the movement states 42 are {-, A, C, G, T, AA, . . . TT} where ‘-’ represents the null sequence fragment. The matrix defining the destination history-state for a given pair of history-state and movement-state might look like:

| — A C G T AA . . . | (SEQ ID NO): |
|---|---|
| AAA AAA AAA AAC AAG AAT AAA | 5 |
| AAC AAC ACA ACC ACG ACT CAA | 6 |
| AAG AAG AGA AGC AGG AGT GAA | 7 |
| AAT AAT ATA ATC ATG ATT TAA | 8 |
| ACA ACA CAA CAC CAG CAT AAA | 9 |

Note that, from a particular history-state 41, there may be several movement-states 42 that give the same destination history-state. This is an expression of the ambiguity that knowledge of the movement-state 42 resolves and differentiates the transducer from something that is only defined on the set of history-states 41 or is defined on the tuple of (source-history-state, destination-history-state), being respectively a Moore machine and a Mealy machine in the parlance of finite-state machines. There is no requirement that the length of longest possible sequence fragment that could be emitted is shorter than the length of the history-state 41.

The posterior probability matrix input into the decoder 37 may be determined by smaller set of parameters, allowing the size of the history-state 41 to be relatively large for the same number of parameters while still allowing flexible emission of sequence fragments from which to assemble the final call.

One example that has proved useful is to have a single weight representing all transitions using the movement corresponding to the empty sequence fragment and all other transitions have a weight that depends solely on the destination history-state. For a history-state-space of fragments of length k and allowed output of up to two bases, this requires 4^{k}+1 parameters rather than the 4^{K}×21 of the complete explicit transducer defined above. Note that this form for transducer only partially resolves the ambiguity that transducers are designed to remove, still needing to make an assumption of maximal but not complete overlap in some cases since scores would be identical; this restriction is often sufficient in many cases that arise in practice when movement-states corresponding to sequence fragments longer than one would rarely be used.

The history-state of the transducer does not have to be over k-mers and could be over some other set of symbols. One example might where the information distinguishing particular bases, purines (A or G) or pyrimidines (C or T), is extremely local and it may advantageous to consider a longer history that cannot distinguish between some bases. For the same number of history-states, a transducer using an alphabet of only purines and pyrimidines could have strings twice as long since 4\hat{ }k=2\hat{ }2k. If P represents a purine Y a pyrimidine, the matrix defining the destination history-state for a given pair of history-state and movement-state would look like:

— A C G T AA . . .
PPP PPP PPP PPY PPP PPY PPP
PPY PPY PYP PYY PYP PYY YPP
PYP PYP YPP YPY YPP YPY PPP
PYY PYY YYP YYY YYP YYY YPP

The history-state 41 of the transducer does not have to be identifiable with one or more fragments of historical sequence and it is advantageous to let the recurrent neural network 30 learn its own representation during training. Given a set of indexed history-states, {S₁, S₂, . . . , S_{H}} and a set of sequence fragments, the movement-states are all possible pairs of a history-state and a sequence fragment. By way of example, the set of sequence fragments may be {-, A, C, G, T, AA, . . . TT} and so the set of movement-states is {S₁-, S₁A, . . . , S₁TT, S₂-, S₂A, . . . , S_{H}TT}. The recurrent neural network 30 emits a posterior probability matrix over these history-states and movement-states as before, each entry representing the posterior probability for moving from one history-state to another by the emission of a particular sequence fragment.

The decoding that is performed by the decoder 37 in the second modification may be performed as follows. In a first application, the decoder may derive an estimate of the series of polymer units from the posterior probability matrices, for example by estimating the most likely path through the posterior probability matrices. The estimate may be an estimate of the series of polymer units as a whole. Details of the decoding are as follows.

Any method known in the art may be used in general, but it is advantageous to use a modification of the Viterbi algorithm to decode a sequence of weights for a transducer into a final sequence. As with the standard Viterbi decoding method, a trace-back matrix is built up during the forwards pass and this used to work out the path taken (assignment of history state to each step) that results in the highest possible score but the transducer modification also requires an additional matrix that records the movement-state actually used in transitioning from one history-state to another along the highest scoring path.

If the weight output by the recurrent neural network 30 at step i for the movement from history-state g via movement-state s is the tensor τᵢₕₛ and the matrix T_{gs} stores the destination history-state then the forwards iteration of the Viterbi algorithm becomes

$${f}_{0 h} = 0 Initialisation$$

$${f}_{ih} = {max}_{s , g s . t . {T}_{gs} = h} {f}_{i - 1 , g} + log {τ}_{igs} Forwards update$$

$${e}_{ih} = {argmax}_{s} {max}_{g s . t . {T}_{gs} = h} {f}_{i - 1 , g} + log {τ}_{igs} Record movement$$

$${b}_{ih} = {argmax}_{g} {max}_{s s . t . {T}_{gs} = h} {f}_{i - 1 , g} + log {τ}_{igs} \text{Trace-back} for history$$

The backwards ‘decoding’ iteration of the modified Viterbi proceeds step-wise from the end. Firstly the last history state for the highest scoring path is determined from the final score vector and then the trace-back information is used to determine all the history states on that path. Once the history-state Hₜ at step t has been determined, the movement-state Mₜ can be determined.

H_{T}=argmaxₕf_{Th}

Hₜ=b_{t,H}_{ₜ₊₁}

Mₜ=e_{t,H}_{ₜ}

Since each movement state has an interpretation as a sequence fragment, the estimate of the polymer sequence can be determined by concatenating these fragments. Since only the movement state is necessary for decoding, the sequence of history-states need never be explicitly determined.

In such a method, the estimation of the most likely path effectively finds as the estimate a series from all possible series that has the highest score representing the probability of the series of polymer units of the polymer being the reference series of polymer units, using the posterior probability matrices. This may be conceptually thought of as scoring against all possible series as references, although in practice the Viterbi algorithm avoids actually scoring every one. More generally, the decoder 37 be arranged to perform other types of analysis that similarly involve generation of a score in respect of one or reference series of polymer units, which score represents the probability of the series of polymer units of the polymer being the reference series of polymer units, using the posterior probability matrices. Such scoring enables several other applications, for example as follows. In the following applications, the reference series of polymer units may be stored in a memory. They may be series of polymer units of known polymers and/or derived from a library or derived experimentally.

In a first alternative, the decoder 36 may derive an estimate of the series of polymer units as a whole by selecting one of a set of plural reference series of polymer units to which the series of posterior probability matrices are most likely to correspond, for example based on scoring the posterior probability matrices against the reference series.

In a second alternative, the decoder 36 may derive an estimate of differences between the series of polymer units of the polymer and a reference series of polymer units. This may be done by scoring variations from the reference series. This effectively estimates the series of polymers from which measurements are taken by estimating the location and identity of differences from the reference. This type of application may be useful, for example, for identifying mutations in a polymer of a known type.

In a third alternative, the estimate may be an estimate of part of the series of polymer units. For example, it may be estimated whether part of the series of polymer units is a reference series of polymer units. This may be done by scoring the reference series against parts of the series of series of posterior probability matrices, for example using a suitable search algorithm. This type of application may be useful, for example, in detecting markers in a polymer.

The third modification also relates to the output of the recurrent neural network 30, and may optionally be combined with the first modification.

One of the limitations of the basic method implemented in the analysis system 3 as described above is the reliance on a decoder 36 external to the recurrent neural network 30 to assign symbols to each column of the output of the recurrent neural network 30 and then estimate the series of polymer units from the sequence of symbols. Since the decoder 36 is not part of the recurrent neural network 30 as such, it must be specified upfront and any parameters cannot be trained along with the rest of network without resorting to complex strategies. In addition, the structure of the Viterbi-style decoder used in the basic method prescribes how the history of the current call is represented and constrains the output of the recurrent neural network 30 itself.

The third modification addresses these limitations and involves changing the output of the recurrent neural network 30 to itself output a decision on the identity of successive polymer units of the series of polymer units. In that case, the decisions are fed back into the recurrent neural network 30, preferably unidirectionally. As a result of being so fed back into the recurrent neural network, the decisions inform the subsequently output decisions.

This modification allows the decoding to be moved from the decoder 36 into the recurrent neural network 30, enabling the decoding process to be trained along with all the other parameters of the recurrent neural network 30 and so optimised to calling from the measurements using nanopore sensing. A further advantage of this third modification is that the representation of history used by the recurrent neural network 30 is learned during training and so adapted to the problem of estimating the series of measurements. By feeding decisions back into the recurrent neural network 30, past decisions can be used by the recurrent neural network 30 to improve prediction of future polymer units.

Several known search methods can be used in conjunction with this method in order to correct past decisions which later appear to be bad. One example of such a method is backtracking, where in response to the recurrent neural network 30 making a low scoring decision, the process rewinds several steps and tries an alternative choice. Another such method is beam search, in which a list of high-scoring history states is kept and at each step the recurrent neural network 30 is used to predict the next polymer unit of the best one.

To illustrate how decoding may be performed, FIG. 14 shows the implementation of the third modification in the final layers of the recurrent neural network 30 and may be compared to FIG. 15 which shows the final layers of the recurrent neural network 30 when implementing the basic method as shown in FIG. 4. Each of FIGS. 14 and 15 show the final bidirectional recurrent layer 34 which by way of non-limitative example has the structure of recurrent units 66 shown in FIG. 8. For brevity in FIGS. 14 and 15, the lines combining the output of the recurrent units 66 with their hidden state before being passed to next recurrent unit 66 are not shown.

However, the final feed-forward layer 35 and the softmax layer 36 of the recurrent neural network 30 shown in FIG. 4 is replaced by a decision layer 45 that outputs decisions on the identity of successive polymer units of the series of polymer units. The decision layer 45 may be implemented by argmax units 46 which each output a respective decision.

Output of decisions, i.e. by the argmax units 46, proceeds sequentially and the final output estimate of the series of polymer units is constructed by appending a new fragment at each step.

Unlike the basic method, each decision is fed back into recurrent neural network 30, in this example being the final bidirectional recurrent layer 34, in particular into the forwards sub-layer 68 (although it could alternatively be the backwards sub-layer 69) thereof. This allows the internal representation of the forwards sub-layer 68 to be informed by the actual decision that has already been produced. The motivation for the feed-back is that there may be several sequences compatible with the input features and straight posterior decoding of the output of a recurrent neural network 30 creates an average of these sequences that is potentially inconsistent and so in general worse that any individual that contributes to it. The feed-back mechanism allows the recurrent neural network 30 to condition its internal state on the actual call being made and so pick out a consistent individual series of in a manner more reminiscent of Viterbi decoding.

The processing is effectively reversible and may proceed in either direction along the polymer, and hence in either direction along the recurrent neural network 30.

The feed-back may be performed by passing each decision (the called symbol) into an embedding unit 47 that emits a vector specific to each symbol.

At each step the output of the lowest bidirectional recurrent layer 34 is projected into the output space, for which each dimension is associated with a fragment of the series of measurements, then argmax decoding is used in the respective argmax units 46 to select the output decision (about the identity of the fragment). The decision is then fed back into the next recurrent unit 66 along in the bidirectional via the embedding unit 47. Every possible decision is associated with a vector in an embedding space and the vector corresponding to the decision just made is combined with the hidden state produced by the current recurrent unit 66 before it is input into the next recurrent unit 66.

By feeding back the decisions into the recurrent neural network 30, the internal representation of the recurrent neural network 30 is informed by both the history of estimated sequence fragments and the measurements. A different formulation of feed back would be where the history of estimated sequence fragments is represented using a separate unidirectional recurrent neural network, the inputs to this recurrent neural network at step is the embedding of the decision and the output is a weight for each decision. These weights are then combined with the weights from processing the measurements in the recurrent neural network before making the argmax decision about the next sequence fragment. Using a separate recurrent neural network in this manner has similarities to the ‘sequence transduction’ method disclosed in Graves, Sequence Transduction with Recurrent Neural Networks, In International Conference on Machine Learning: Representation Learning Workshop, 2012 and is a special case of the third modification.

The parameters of the recurrent unit 66 into which the embedding of the decision is fed back are constrained so that its state is factored to two parts whose updates are only dependent on either the output of the upper layers of the recurrent neural network 30 prior to the final bidirectional recurrent layer 34 or embedded decisions.

Training of the third modification may be performed as follows.

To make output of the recurrent neural network 30 compatible with training using the perplexity, or other probability or entropy based objective functions, the recurrent neural network 30 shown in FIG. 14 may be adapted for the purpose of training as shown in either of FIG. 16 or 17 by the addition of softmax units 48. The softmax units 48 apply the softmax functor to the output (the softmax unit being as previously described but without applying an affine transform) of the final bidirectional recurrent layer 34. Then training is performed on the output of the softmax units 48 by perplexity as shown by elements 49. In the example of FIG. 16, the softmax units 48 replace the argmax units 46 and the training labels output by softmax units 48 are fed back, whereas in the example of FIG. 17, the softmax units 48 are arranged in parallel with the argmax units 46 and the decisions output by the argmax units 46 are fed back.

Rather than the hard decisions about the fragment of the series of polymers made by the argmax units 46, the softmax units 48 create outputs that can be interpreted as a probability distribution over fragments of the series of polymer and so are trainable by perplexity. Since the softmax functor implemented in the softmax units 48 preserves the order of its inputs, the argmax of this unit is the same as what would have been obtained if it had not been added to the recurrent neural network 30. Even when the recurrent neural network 30 has been trained it can be advantageous to leave the softmax unit in the recurrent neural network 30 since it provides a measure of confidence in the decision.

The dependence of the recurrent neural network 30 on its output up to a given step poses problems for training since a change in parameters that causes the output decision at any step to change requires crossing a non-differentiable boundary and optimisation can be difficult. One way to avoid problems with non-differentiability is to train the recurrent neural network 30 using the perplexity objective but pretend that the call was perfect up to that point, feeding the training label to the embedding units 47 rather than the decision that would have been made. Training in this manner produces a network that performs fine provided the sequence fragment call are correct but may be extremely sensitive to errors since it has not been trained to recover from a poor call.

Training may be performed with a two-stage approach. Firstly the training labels are fed back into the recurrent neural network 30, as described above and shown in FIG. 16.

Secondly the actual calls made are fed back in but still calculating perplexity via a softmax unit 48, as shown in FIG. 17. The motivation for this two stage process is that the first stage finds good starting parameters for the second stage, thereby reducing the chance that training gets stuck in a bad parameter region because of the afore mentioned non-differentiability.

The invention will now be further described by the following non-limiting examples.

### EXAMPLES

Protocol for PCA Ligation:

1000 ng of target DNA was end-repaired and dA-tailed before being ligated to PCA from PCR Sequencing kit (SQK-PSK004).

All reactions and purifications were carried out according to the manufacturer's instructions; NEB for the end-repair and dA-tailing and ONT for ligation.

Protocol for 1× Cycle Amplification:

50 ul reactions consisted of; 250 ng PCA ligated target DNA, 1× ThermoPol Buffer (NEB), 200 nM Primer, 400 uM dNTPs, 0.1 unit ul-1 9oNm Polymerase.

Primer used was WGP from Oxford Nanopore's PCR Sequencing kit (SQK-PSK004).

Cycled accordingly; 95oC for 45 secs, 56° C. for 45 secs, 68° C. for 35 min.

After amplification, 10 units of Exonuclease I (NEB) was added and samples were then incubated for a further 15 mins at 37° C.

Samples were purified using Beckman Coulters Agencourt AMPure XP beads (0.4×) and eluted into 30 ul of TE.

Protocol for Sequencing Adapter Attachment:

Recovered amplified target DNA was mixed with RAP, LLB and SQB before being loaded onto a R9.4.1 Flowcell (FLO-MIN106).

All steps were performed using Oxford Nanopore's PCR Sequencing kit (SQK-PSK004) following manufacturer's instructions.

### Example 1

Polynucleotide strands of approximately 3.6 kb in length and comprising either canonical bases only or a mixture of canonical and non-canonical bases were generated and amplified using the above protocols.

A control strand was generated composed only of the canonical bases G, T, A and C; see FIG. 1 and accompanying legend. Additional test strands were generated with differing proportions of non-canonical bases; see FIGS. 2-7 and accompanying legends.

The control and test strands were subjected to nanopore sequencing. The modified strands could be differentiated from the control strands based on the current traces obtained; see FIGS. 11 and 12 and accompanying legends.

### Example 2

An E. coli library was subjected to two separate amplifications: one amplification using the canonical bases G, T, A and C; and one amplification using non-canonical bases. See FIGS. 9-10 and accompanying legends. Amplification was successful in both cases, demonstrating the ability to amplify a library using non-canonical bases.

1. A method of determining a sequence of a target polymer, or part thereof, comprising polymer units comprising canonical and non-canonical polymer units, the method comprising:
taking a series of measurements of a signal relating to the target polymer, wherein a measurement of the signal is dependent upon a plurality of polymer units, and wherein the polymer units of the target polymer modulate the signal, and wherein a non-canonical polymer unit modulates the signal differently from a corresponding canonical polymer unit;
analysing the series of measurements using a machine learning technique that attributes a measurement of a non-canonical polymer unit to being a measurement of a respective corresponding canonical polymer unit; and
determining the sequence of the target polymer, or part thereof, from the analysed series of measurements.

2. A method according to claim 1, wherein a non-canonical polymer unit identified from the analysis is additionally or alternatively determined.

3. A method of claim 1, wherein the target polymer comprises two or more types of non-canonical polymer units corresponding to the two or more types of canonical polymer unit.

4. A method according to claim 1, wherein the identity and sequence position of a non-canonical polymer unit is determined.

5. A method according claim 1, wherein the target polymer comprises non-canonical polymer units corresponding to each type of canonical polymer unit.

6. A method according to claim 1, wherein the machine learning technique does not determine between whether a polymer unit is non-canonical or a corresponding canonical polymer unit

7. The method according to claim 1 wherein, the target polymer comprises plural non-canonical polymer units for each of the one or more types of non-canonical polymer unit present.

8. A method according to claim 1, wherein a non-canonical polymer unit may correspond to more than one canonical polymer unit.

9. A method according to claim 1, wherein the target polymer comprises approximately 50% of non-canonical polymer units.

10. A method according to claim 1, wherein a non-canonical polymer unit is a modified canonical polymer unit.

11. A method according to claim 1, wherein the non-canonical polymer unit is naturally modified.

12. A method according to claim 1, wherein the series of measurements are taken during movement of the target polymer with respect to a nanopore.

13. A method according to claim 1, wherein the measurements are measurements indicative of ion current flow through the nanopore or measurements of a voltage across the nanopore during translocation of the target polymer.

14. A method according to claim 1 wherein the machine learning technique is trainable by a method comprising the steps of:
providing a plurality of target polymers comprising non-canonical units that have been substituted for equivalent canonical units at varying sequence positions in the target polymer;
taking series of measurements of signals relating to the target polymers;
analysing the series of measurements using the machine learning technique; and
estimating the corresponding canonical polymer units of the polymer training strands.

15. (canceled)

16. A method according to claim 1 wherein the polymer is a polynucleotide and the polymer units are nucleotide bases.

17. A method according to claim 1, wherein the one or more non-canonical bases has been modified by means of an enzyme.

18. A method according to claim 1, further comprising the step of modifying a canonical polymer to provide the target polymer comprising one or more one or more non-canonical bases of one or more different types.

19. A method according to claim 1, wherein the polynucleotide comprising one or more non-canonical bases of one or more different types is generated from its complement by use of a polymerase and a proportion of non-canonical bases.

20. A method according to claim 1 wherein the polynucleotide is DNA.

21-22. (canceled)

23. A method according to claim 14, wherein a polynucleotide training strand comprises more than one type of non-canonical polymer unit.

24-42. (canceled)
